SEQUENCE LISTING (66 sequences)

SEQ ID NO: 1 - protein, 175 aa, Artificial sequence
  amino acid sequence for filgrastim
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 2 - protein, 207 aa, Homo sapiens
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 3 - protein, 204 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 4 - protein, 171 aa, Homo sapiens
MAGPATQSPMKLMALQLLLWHSALWTVQEATPLGPASSLPQSFLLKCLEQVRKIQGDGAALQEKLVSEAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 5 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 6 - protein, 273 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 7 - DNA, 516 bp, Homo sapiens
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtga

SEQ ID NO: 8 - DNA, 624 bp, Homo sapiens
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctga

SEQ ID NO: 9 - DNA, 549 bp, Homo sapiens
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaattga

SEQ ID NO: 10 - DNA, 495 bp, Homo sapiens
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 11 - DNA, 1128 bp, Homo sapiens
atggagcgcgcgtcctgcttgttgctgctgctgctgccgctggtgcacgtctctgcgaccacgccagaaccttgtgagctggacgatgaagatttccgctgcgtctgcaacttctccgaacctcagcccgactggtccgaagccttccagtgtgtgtctgcagtagaggtggagatccatgccggcggtctcaacctagagccgtttctaaagcgcgtcgatgcggacgccgacccgcggcagtatgctgacacggtcaaggctctccgcgtgcggcggctcacagtgggagccgcacaggttcctgctcagctactggtaggcgccctgcgtgtgctagcgtactcccgcctcaaggaactgacgctcgaggacctaaagataaccggcaccatgcctccgctgcctctggaagccacaggacttgcactttccagcttgcgcctacgcaacgtgtcgtgggcgacagggcgttcttggctcgccgagctgcagcagtggctcaagccaggcctcaaggtactgagcattgcccaagcacactcgcctgccttttcctgcgaacaggttcgcgccttcccggcccttaccagcctagacctgtctgacaatcctggactgggcgaacgcggactgatggcggctctctgtccccacaagttcccggccatccagaatctagcgctgcgcaacacaggaatggagacgcccacaggcgtgtgcgccgcactggcggcggcaggtgtgcagccccacagcctagacctcagccacaactcgctgcgcgccaccgtaaaccctagcgctccgagatgcatgtggtccagcgccctgaactccctcaatctgtcgttcgctgggctggaacaggtgcctaaaggactgccagccaagctcagagtgctcgatctcagctgcaacagactgaacagggcgccgcagcctgacgagctgcccgaggtggataacctgacactggacgggaatcccttcctggtccctggaactgccctcccccacgagggctcaatgaactccggcgtggtcccagcctgtgcacgttcgaccctgtcggtgggggtgtcgggaaccctggtgctgctccaaggggcccggggctttgcctaa

SEQ ID NO: 12 - DNA, 765 bp, Homo sapiens
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 13 - DNA, 702 bp, Homo sapiens
atgtggcagctgctcctcccaactgctctgctacttctagtttcagctggcatgcggactgaagatctcccaaaggctgtggtgttcctggagcctcaatggtacagcgtgcttgagaaggacagtgtgactctgaagtgccagggagcctactcccctgaggacaattccacacagtggtttcacaatgagaacctcatctcaagccaggcctcgagctacttcattgacgctgccacagtcaacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgcagtggacacaggactatatttctctgtgaagacaaacatttga

SEQ ID NO: 14 - DNA, 1671 bp, Homo sapiens
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtga

SEQ ID NO: 15 - DNA, 1158 bp, Homo sapiens
atgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctgggcgaagacccttattacacggaaaacggtggaggccagggctatagctcaggacctgggacctcccctgaggctcagggaaaggccagtgtgaaccgaggggctcaggaaaacgggaccggccaggccacctccagaaacggccattcagcaagacaacacgtggtggctgataccgaattgtga

SEQ ID NO: 16 - DNA, 987 bp, Homo sapiens
atgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctggagctggaaccctga

SEQ ID NO: 17 - DNA, 3921 bp, Homo sapiens
atgaccatgtatttgtggcttaaactcttggcatttggctttgcctttctggacacagaagtatttgtgacagggcaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaatttagatgaacagcaggagcttgttgaaagggatgatgaaaaacaactgatgaatgtggagccaatccatgcagatattttgttggaaacttataagaggaagattgctgatgaaggaagactttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgttgttgtaaagatcaaccagcacaaaagatgtccagattacatcattcagaaattgaacattgtaaataaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcagtgctggtgttgggcgcacaggaacctatatcggaattgatgccatgctagaaggcctggaagccgagaacaaagtggatgtttatggttatgttgtcaagctaaggcgacagagatgcctgatggttcaagtagaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccagagacttccttcatataggagctggaggacacagcacattggaaatcaagaagaaaataaaagtaaaaacaggaattctaatgtcatcccatatgactataacagagtgccacttaaacatgagctggaaatgagtaaagagagtgagcatgattcagatgaatcctctgatgatgacagtgattcagaggaaccaagcaaatacatcaatgcatcttttataatgagctactggaaacctgaagtgatgattgctgctcagggaccactgaaggagaccattggtgacttttggcagatgatcttccaaagaaaagtcaaagttattgttatgctgacagaactgaaacatggagaccaggaaatctgtgctcagtactggggagaaggaaagcaaacatatggagatattgaagttgacctgaaagacacagacaaatcttcaacttatacccttcgtgtctttgaactgagacattccaagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcagggatggatctcagcaaacgggaatattttgtgctttgttaaatctcttagaaagtgcggaaacagaagaggtagtggatatttttcaagtggtaaaagctctacgcaaagctaggccaggcatggtttccacattcgagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcatag

SEQ ID NO: 18 - DNA, 3438 bp, Homo sapiens
atgaccatgtatttgtggcttaaactcttggcatttggctttgcctttctggacacagaagtatttgtgacagggcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaatttagatgaacagcaggagcttgttgaaagggatgatgaaaaacaactgatgaatgtggagccaatccatgcagatattttgttggaaacttataagaggaagattgctgatgaaggaagactttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgttgttgtaaagatcaaccagcacaaaagatgtccagattacatcattcagaaattgaacattgtaaataaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcagtgctggtgttgggcgcacaggaacctatatcggaattgatgccatgctagaaggcctggaagccgagaacaaagtggatgtttatggttatgttgtcaagctaaggcgacagagatgcctgatggttcaagtagaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccagagacttccttcatataggagctggaggacacagcacattggaaatcaagaagaaaataaaagtaaaaacaggaattctaatgtcatcccatatgactataacagagtgccacttaaacatgagctggaaatgagtaaagagagtgagcatgattcagatgaatcctctgatgatgacagtgattcagaggaaccaagcaaatacatcaatgcatcttttataatgagctactggaaacctgaagtgatgattgctgctcagggaccactgaaggagaccattggtgacttttggcagatgatcttccaaagaaaagtcaaagttattgttatgctgacagaactgaaacatggagaccaggaaatctgtgctcagtactggggagaaggaaagcaaacatatggagatattgaagttgacctgaaagacacagacaaatcttcaacttatacccttcgtgtctttgaactgagacattccaagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcagggatggatctcagcaaacgggaatattttgtgctttgttaaatctcttagaaagtgcggaaacagaagaggtagtggatatttttcaagtggtaaaagctctacgcaaagctaggccaggcatggtttccacattcgagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcatag

SEQ ID NO: 19 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 20 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 21 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 22 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 23 - protein, 375 aa, Homo sapiens
MERASCLLLLLLPLVHVSATTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMNSGVVPACARSTLSVGVSGTLVLLQGARGFA

SEQ ID NO: 24 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 25 - protein, 233 aa, Homo sapiens
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 26 - protein, 556 aa, Homo sapiens [sequence omitted]

SEQ ID NO: 27 - protein, 385 aa, Homo sapiens
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLGEDPYYTENGGGQGYSSGPGTSPEAQGKASVNRGAQENGTGQATSRNGHSARQHVVADTEL

SEQ ID NO: 28 - protein, 328 aa, Homo sapiens
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEP

SEQ ID NO: 29 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 30 - protein, 1145 aa, Homo sapiens
MTMYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 31 - DNA, 4368 bp, Homo sapiens
atgtccgactcgtgggtcccgaactccgcctcgggccaggacccagggggccgccggagggcctgggccgagctgctggcgggaagggtcaagagggaaaaatataatcctgaaagggcacagaaattaaaggaatcagctgtgcgcctcctgcgaagccatcaggacctgaatgcccttttgcttgaggtagaaggtccactgtgtaaaaaattgtctctcagcaaagtgattgactgtgacagttctgaggcctatgctaatcattctagttcatttataggctctgctttgcaggatcaagcctcaaggctgggggttcccgtgggtattctctcagccgggatggttgcctctagcgtgggacagatctgcacggctccagcggagaccagtcaccctgtgctgctgactgtggagcagagaaagaagctgtcttccctgttagagtttgctcagtatttattggcacacagtatgttctcccgtctttccttctgtcaagaattatggaaaatacagagttctttgttgcttgaagcggtgtggcatcttcacgtacaaggcattgtgagcctgcaagagctgctggaaagccatcccgacatgcatgctgtgggatcgtggctcttcaggaatctgtgctgcctttgtgaacagatggaagcatcctgccagcatgctgacgtcgccagggccatgctttctgattttgttcaaatgtttgttttgaggggatttcagaaaaactcagatctgagaagaactgtggagcctgaaaaaatgccgcaggtcacggttgatgtactgcagagaatgctgatttttgcacttgacgctttggctgctggagtacaggaggagtcctccactcacaagatcgtgaggtgctggttcggagtgttcagtggacacacgcttggcagtgtaatttccacagatcctctgaagaggttcttcagtcataccctgactcagatactcactcacagccctgtgctgaaagcatctgatgctgttcagatgcagagagagtggagctttgcgcggacacaccctctgctcacctcactgtaccgcaggctctttgtgatgctgagtgcagaggagttggttggccatttgcaagaagttctggaaacgcaggaggttcactggcagagagtgctctcctttgtgtctgccctggttgtctgctttccagaagcgcagcagctgcttgaagactgggtggcgcgtttgatggcccaggcattcgagagctgccagctggacagcatggtcactgcgttcctggttgtgcgccaggcagcactggagggcccctctgcgttcctgtcatatgcagactggttcaaggcctcctttgggagcacacgaggctaccatggctgcagcaagaaggccctggtcttcctgtttacgttcttgtcagaactcgtgccttttgagtctccccggtacctgcaggtgcacattctccacccacccctggttcccagcaagtaccgctccctcctcacagactacatctcattggccaagacacggctggccgacctcaaggtttctatagaaaacatgggactctacgaggatttgtcatcagctggggacattactgagccccacagccaagctcttcaggatgttgaaaaggccatcatggtgtttgagcatacggggaacatcccagtcaccgtcatggaggccagcatattcaggaggccttactacgtgtcccacttcctccccgccctgctcacacctcgagtgctccccaaagtccctgactcccgtgtggcgtttatagagtctctgaagagagcagataaaatccccccatctctgtactccacctactgccaggcctgctctgctgctgaagagaagccagaagatgcagccctgggagtgagggcagaacccaactctgctgaggagcccctgggacagctcacagctgcactgggagagctgagagcctccatgacagaccccagccagcgtgatgttatatcggcacaggtggcagtgatttctgaaagactgagggctgtcctgggccacaatgaggatgacagcagcgttgagatatcaaagattcagctcagcatcaacacgccgagactggagccacgggaacacattgctgtggacctcctgctgacgtctttctgtcagaacctgatggctgcctccagtgtcgctcccccggagaggcagggtccctgggctgccctcttcgtgaggaccatgtgtggacgtgtgctccctgcagtgctcacccggctctgccagctgctccgtcaccagggcccgagcctgagtgccccacatgtgctggggttggctgccctggccgtgcacctgggtgagtccaggtctgcgctcccagaggtggatgtgggtcctcctgcacctggtgctggccttcctgtccctgcgctctttgacagcctcctgacctgtaggacgagggattccttgttcttctgcctgaaattttgtacagcagcaatttcttactctctctgcaagttttcttcccagtcacgagatactttgtgcagctgcttatctccaggccttattaaaaagtttcagttcctcatgttcagattgttctcagaggcccgacagcctctttctgaggaggacgtagccagcctttcctggagacccttgcaccttccttctgcagactggcagagagctgccctctctctctggacacacagaaccttccgagaggtgttgaaagaggaagatgttcacttaacttaccaagactggttacacctggagctggaaattcaacctgaagctgatgctctttcagatactgaacggcaggacttccaccagtgggcgatccatgagcactttctccctgagtcctcggcttcagggggctgtgacggagacctgcaggctgcgtgtaccattcttgtcaacgcactgatggatttccaccaaagctcaaggagttatgaccactcagaaaattctgatttggtctttggtggccgcacaggaaatgaggatattatttccagattgcaggagatggtagctgacctggagctgcagcaagacctcatagtgcctctcggccacaccccttcccaggagcacttcctctttgagattttccgcagacggctccaggctctgacaagcgggtggagcgtggctgccagccttcagagacagagggagctgctaatgtacaaacggatcctcctccgcctgccttcgtctgtcctctgcggcagcagcttccaggcagaacagcccatcactgccagatgcgagcagttcttccacttggtcaactctgagatgagaaacttctgctcccacggaggtgccctgacacaggacatcactgcccacttcttcaggggcctcctgaacgcctgtctgcggagcagagacccctccctgatggtcgacttcatactggccaagtgccagacgaaatgccccttaattttgacctctgctctggtgtggtggccgagcctggagcctgtgctgctctgccggtggaggagacactgccagagcccgctgccccgggaactgcagaagctacaagaaggccggcagtttgccagcgatttcctctcccctgaggctgcctccccagcacccaacccggactggctctcagctgctgcactgcactttgcgattcaacaagtcagggaagaaaacatcaggaagcagctaaagaagctggactgcgagagagaggagctattggttttccttttcttcttctccttgatgggcctgctgtcgtcacatctgacctcaaatagcaccacagacctgccaaaggctttccacgtttgtgcagcaatcctcgagtgtttagagaagaggaagatatcctggctggcactctttcagttgacagagagtgacctcaggctggggcggctcctcctccgtgtggccccggatcagcacaccaggctgctgcctttcgctttttacagtcttctctcctacttccatgaagacgcggccatcagggaagaggccttcctgcatgttgctgtggacatgtacttgaagctggtccagctcttcgtggctggggatacaagcacagtttcacctccagctggcaggagcctggagctcaagggtcagggcaaccccgtggaactgataacaaaagctcgtctttttctgctgcagttaatacctcggtgcccgaaaaagagcttctcacacgtggcagagctgctggctgatcgtggggactgcgacccagaggtgagcgccgccctccagagcagacagcaggctgcccctgacgctgacctgtcccaggagcctcatctcttctga

SEQ ID NO: 32 - DNA, 1677 bp, Homo sapiens
atggctcaagattcagtagatctttcttgtgattatcagttttggatgcagaagctttctgtatgggatcaggcttccactttggaaacccagcaagacacctgtcttcacgtggctcagttccaggagttcctaaggaagatgtatgaagccttgaaagagatggattctaatacagtcattgaaagattccccacaattggtcaactgttggcaaaagcttgttggaatccttttattttagcatatgatgaaagccaaaaaattctaatatggtgcttatgttgtctaattaacaaagaaccacagaattctggacaatcaaaacttaactcctggatacagggtgtattatctcatatactttcagcactcagatttgataaagaagttgctcttttcactcaaggtcttgggtatgcacctatagattactatcctggtttgcttaaaaatatggttttatcattagcgtctgaactcagagagaatcatcttaatggatttaacactcaaaggcgaatggctcccgagcgagtggcgtccctgtcacgagtttgtgtcccacttattaccctgacagatgttgaccccctggtggaggctctcctcatctgtcatggacgtgaacctcaggaaatcctccagccagagttctttgaggctgtaaacgaggccattttgctgaagaagatttctctccccatgtcagctgtagtctgcctctggcttcggcaccttcccagccttgaaaaagcaatgctgcatctttttgaaaagctaatctccagtgagagaaattgtctgagaaggatcgaatgctttataaaagattcatcgctgcctcaagcagcctgccaccctgccatattccgggttgttgatgagatgttcaggtgtgcactcctggaaaccgatggggccctggaaatcatagccactattcaggtgtttacgcagtgctttgtagaagctctggagaaagcaagcaagcagctgcggtttgcactcaagacctactttccttacacttctccatctcttgccatggtgctgctgcaagaccctcaagatatccctcggggacactggctccagacactgaagcatatttctgaactgctcagagaagcagttgaagaccagactcatgggtcctgcggaggtccctttgagagctggttcctgttcattcacttcggaggatgggctgagatggtggcagagcaattactgatgtcggcagccgaaccccccacggccctgctgtggctcttggccttctactacggcccccgtgatgggaggcagcagagagcacagactatggtccaggtgaaggccgtgctgggccacctcctggcaatgtccagaagcagcagcctctcagcccaggacctgcagacggtagcaggacagggcacagacacagacctcagagctcctgcacaacagctgatcaggcaccttctcctcaacttcctgctctgggctcctggaggccacacgatcgcctgggatgtcatcaccctgatggctcacactgctgagataactcacgagatcattggctttcttgaccagaccttgtacagatggaatcgtcttggcattgaaagccctagatcagaaaaactggcccgagagctccttaaagagctgcgaactcaagtctag

SEQ ID NO: 33 - DNA, 1611 bp, Homo sapiens
atggcgacaccggacgcggggctccctggggctgagggcgtggagccggcgccctgggcgcagctggaggcccccgcccgcctcctgctgcaggcgctgcaggcggggcctgagggggcgcggcgcggcctgggggtgctccgggcgctgggcagccgcggctgggagcccttcgactggggtcgcttgctcgaggccctgtgccgggaggagccggtcgtgcaggggcctgacggccgtctggagctgaaaccactgttgctgcgattgccccggatatgccagaggaacctgatgtccctgctgatggccgttcggccatcgctgccggaaagtgggctcctctctgtgctgcagattgcccagcaggacctagcccctgacccagatgcctggctccgtgccctgggggaattgctgcgaagggatttgggggtggggacctccatggagggagcttctccactgtctgaaagatgccagagacagctccaaagtctatgtagggggctgggcctggggggcaggaggttgaaatccccccaggctccagaccctgaagaagaggagaacagggactcccagcagcctgggaaacgcagaaaggactcagaggaagaggctgccagtcctgaggggaagagggtccccaaaagattacggtgttgggaagaggaagaagatcatgagaaggagagacccgaacataagtcactggaatccctggcagatggaggaagtgcatctcctattaaggaccagcctgtcatggcagttaagactggcgaggacggttcgaatctggatgatgctaaaggtctggctgagagtttggagttgcccaaagctatccaggaccagcttcccaggctgcagcagctgctgaagaccttggaggaggggttagagggattggaggatgcccccccagttgagctacagcttcttcacgaatgtagtcccagccagatggacttgctgtgtgcccagctgcagctccctcagctctcagacctcggtctcctgcggctctgcacctggctgctggccctttcacctgatctcagcctcagcaatgctactgtgctgaccagaagcctctttcttggacggatcctctccttgacttcctcagcctcccgcctgcttacaactgccctgacctccttctgtgccaaatatacataccctgtctgcagcgccctccttgaccctgtgctccaggccccaggcacaggtcctgctcaaacagagttactgtgttgccttgtgaagatggagtccctggagccagatgcacaggttctaatgctgggacagatcttggagctgccctggaaggaggaaactttcttggtgttgcagtcactcctagagcggcaggtggagatgacccctgagaagttcagtgtcttaatggagaagctctgtaaaaaggggctggcagccaccacctccatggcctatgccaagctcatgctgacagtgatgaccaagtatcaggctaacatcactgagacccagaggctgggcctggctatggccctagaacctaacaccaccttcctgaggaagtccctgaaggccgccttgaaacatttgggcccctga

SEQ ID NO: 34 - DNA, 1125 bp, Homo sapiens
atggaatcccttctgcagcacctggatcgcttttccgagcttctggcggtctcaagcactacctacgtcagcacctgggaccccgccaccgtgcgccgggccttgcagtgggcgcgctacctgcgccacatccatcggcgctttggtcggcatggccccattcgcacggctctggagcggcggctgcacaaccagtggaggcaagagggcggctttgggcggggtccagttccgggattagcgaacttccaggccctcggtcactgtgacgtcctgctctctctgcgcctgctggagaaccgggccctcggggatgcagctcgttaccacctggtgcagcaactctttcccggcccgggcgtccgggacgccgatgaggagacactccaagagagcctggcccgccttgcccgccggcggtctgcggtgcacatgctgcgcttcaatggctatagagagaacccaaatctccaggaggactctctgatgaagacccaggcggagctgctgctggagcgtctgcaggaggtggggaaggccgaagcggagcgtcccgccaggtttctcagcagcctgtgggagcgcttgcctcagaacaacttcctgaaggtgatagcggtggcgctgttgcagccgcctttgtctcgtcggccccaagaagagttggaacccggcatccacaaatcacctggagaggggagccaagtgctagtccactggcttctggggaattcggaagtctttgctgccttttgtcgcgccctcccagccgggcttttgactttagtgactagccgccacccagcgctgtctcctgtctatctgggtctgctaacagactggggtcaacgtttgcactatgaccttcagaaaggcatttgggttggaactgagtcccaagatgtgccctgggaggagttgcacaataggtttcaaagcctctgtcaggcccctccacctctgaaagataaagttctaactgccctggagacctgtaaagcgcaggatggagattttgaagtacctggtcttagcatctggacagacctcttattagctcttcgtagtggtgcatttaggaaaagacaagttttgggtctcagcgcaggcctcagttctgtatag

SEQ ID NO: 35 - DNA, 1869 bp, Homo sapiens
atgtcccgccagaccacctctgtgggctccagctgcctggacctgtggagggaaaagaatgaccggctcgttcgacaggccaaggtggctcagaactccggtctgactctgaggcgacagcagttggctcaggatgcactggaagggctcagagggctcctccatagtctgcaagggctccctgcagctgttcctgttcttcccttggagctgactgtcacctgcaacttcattatcctgagggcaagcttggcccagggtttcacagaggatcaggcccaggatatccagcggagcctagagagagtgctggagacacaggagcagcaggggcccaggttggaacaggggctcagggagctgtgggactctgtccttcgtgcttcctgccttctgccggagctgctgtctgccctgcaccgcctggttggcctgcaggctgccctctggttgagtgctgaccgtcttggggacctggccttgttactagagaccctgaatggcagccagagtggagcctctaaggatctgctgttacttctgaaaacttggagtcccccagctgaggaattagatgctccattgaccctgcaggatgcccagggattgaaggatgtcctcctgacagcatttgcctaccgccaaggtctccaggagctgatcacagggaacccagacaaggcactaagcagccttcatgaagcggcctcaggcctgtgtccacggcctgtgttggtccaggtgtacacagcactggggtcctgtcaccgtaagatgggaaatccacagagagcactgttgtacttggttgcagccctgaaagagggatcagcctggggtcctccacttctggaggcctctaggctctatcagcaactgggggacacaacagcagagctggagagtctggagctgctagttgaggccttgaatgtcccatgcagttccaaagccccgcagtttctcattgaggtagaattactactgccaccacctgacctagcctcaccccttcattgtggcactcagagccagaccaagcacatactagcaagcaggtgcctacagacggggagggcaggagacgctgcagagcattacttggacctgctggccctgttgctggatagctcggagccaaggttctccccacccccctcccctccagggccctgtatgcctgaggtgtttttggaggcagcggtagcactgatccaggcaggcagagcccaagatgccttgactctatgtgaggagttgctcagccgcacatcatctctgctacccaagatgtcccggctgtgggaagatgccagaaaaggaaccaaggaactgccatactgcccactctgggtctctgccacccacctgcttcagggccaggcctgggttcaactgggtgcccaaaaagtggcaattagtgaatttagcaggtgcctcgagctgctcttccgggccacacctgaggaaaaagaacaaggggcagctttcaactgtgagcagggatgtaagtcagatgcggcactgcagcagcttcgggcagccgccctaattagtcgtggactggaatgggtagccagcggccaggataccaaagccttacaggacttcctcctcagtgtgcagatgtgcccaggtaatcgagacacttactttcacctgcttcagactctgaagaggctagatcggagggatgaggccactgcactctggtggaggctggaggcccaaactaaggggtcacatgaagatgctctgtggtctctccccctgtacctagaaagctatttgagctggatccgtccctctgatcgtgacgccttccttgaagaatttcggacatctctgccaaagtcttgtgacctgtag

SEQ ID NO: 36 - protein, 1455 aa, Homo sapiens
MSDSWVPNSASGQDPGGRRRAWAELLAGRVKREKYNPERAQKLKESAVRLLRSHQDLNALLLEVEGPLCKKLSLSKVIDCDSSEAYANHSSSFIGSALQDQASRLGVPVGILSAGMVASSVGQICTAPAETSHPVLLTVEQRKKLSSLLEFAQYLLAHSMFSRLSFCQELWKIQSSLLLEAVWHLHVQGIVSLQELLESHPDMHAVGSWLFRNLCCLCEQMEASCQHADVARAMLSDFVQMFVLRGFQKNSDLRRTVEPEKMPQVTVDVLQRMLIFALDALAAGVQEESSTHKIVRCWFGVFSGHTLGSVISTDPLKRFFSHTLTQILTHSPVLKASDAVQMQREWSFARTHPLLTSLYRRLFVMLSAEELVGHLQEVLETQEVHWQRVLSFVSALVVCFPEAQQLLEDWVARLMAQAFESCQLDSMVTAFLVVRQAALEGPSAFLSYADWFKASFGSTRGYHGCSKKALVFLFTFLSELVPFESPRYLQVHILHPPLVPSKYRSLLTDYISLAKTRLADLKVSIENMGLYEDLSSAGDITEPHSQALQDVEKAIMVFEHTGNIPVTVMEASIFRRPYYVSHFLPALLTPRVLPKVPDSRVAFIESLKRADKIPPSLYSTYCQACSAAEEKPEDAALGVRAEPNSAEEPLGQLTAALGELRASMTDPSQRDVISAQVAVISERLRAVLGHNEDDSSVEISKIQLSINTPRLEPREHIAVDLLLTSFCQNLMAASSVAPPERQGPWAALFVRTMCGRVLPAVLTRLCQLLRHQGPSLSAPHVLGLAALAVHLGESRSALPEVDVGPPAPGAGLPVPALFDSLLTCRTRDSLFFCLKFCTAAISYSLCKFSSQSRDTLCSCLSPGLIKKFQFLMFRLFSEARQPLSEEDVASLSWRPLHLPSADWQRAALSLWTHRTFREVLKEEDVHLTYQDWLHLELEIQPEADALSDTERQDFHQWAIHEHFLPESSASGGCDGDLQAACTILVNALMDFHQSSRSYDHSENSDLVFGGRTGNEDIISRLQEMVADLELQQDLIVPLGHTPSQEHFLFEIFRRRLQALTSGWSVAASLQRQRELLMYKRILLRLPSSVLCGSSFQAEQPITARCEQFFHLVNSEMRNFCSHGGALTQDITAHFFRGLLNACLRSRDPSLMVDFILAKCQTKCPLILTSALVWWPSLEPVLLCRWRRHCQSPLPRELQKLQEGRQFASDFLSPEAASPAPNPDWLSAAALHFAIQQVREENIRKQLKKLDCEREELLVFLFFFSLMGLLSSHLTSNSTTDLPKAFHVCAAILECLEKRKISWLALFQLTESDLRLGRLLLRVAPDQHTRLLPFAFYSLLSYFHEDAAIREEAFLHVAVDMYLKLVQLFVAGDTSTVSPPAGRSLELKGQGNPVELITKARLFLLQLIPRCPKKSFSHVAELLADRGDCDPEVSAALQSRQQAAPDADLSQEPHLF

SEQ ID NO: 37 - protein, 558 aa, Homo sapiens
MAQDSVDLSCDYQFWMQKLSVWDQASTLETQQDTCLHVAQFQEFLRKMYEALKEMDSNTVIERFPTIGQLLAKACWNPFILAYDESQKILIWCLCCLINKEPQNSGQSKLNSWIQGVLSHILSALRFDKEVALFTQGLGYAPIDYYPGLLKNMVLSLASELRENHLNGFNTQRRMAPERVASLSRVCVPLITLTDVDPLVEALLICHGREPQEILQPEFFEAVNEAILLKKISLPMSAVVCLWLRHLPSLEKAMLHLFEKLISSERNCLRRIECFIKDSSLPQAACHPAIFRVVDEMFRCALLETDGALEIIATIQVFTQCFVEALEKASKQLRFALKTYFPYTSPSLAMVLLQDPQDIPRGHWLQTLKHISELLREAVEDQTHGSCGGPFESWFLFIHFGGWAEMVAEQLLMSAAEPPTALLWLLAFYYGPRDGRQQRAQTMVQVKAVLGHLLAMSRSSSLSAQDLQTVAGQGTDTDLRAPAQQLIRHLLLNFLLWAPGGHTIAWDVITLMAHTAEITHEIIGFLDQTLYRWNRLGIESPRSEKLARELLKELRTQV

SEQ ID NO: 38 - protein, 536 aa, Homo sapiens
MATPDAGLPGAEGVEPAPWAQLEAPARLLLQALQAGPEGARRGLGVLRALGSRGWEPFDWGRLLEALCREEPVVQGPDGRLELKPLLLRLPRICQRNLMSLLMAVRPSLPESGLLSVLQIAQQDLAPDPDAWLRALGELLRRDLGVGTSMEGASPLSERCQRQLQSLCRGLGLGGRRLKSPQAPDPEEEENRDSQQPGKRRKDSEEEAASPEGKRVPKRLRCWEEEEDHEKERPEHKSLESLADGGSASPIKDQPVMAVKTGEDGSNLDDAKGLAESLELPKAIQDQLPRLQQLLKTLEEGLEGLEDAPPVELQLLHECSPSQMDLLCAQLQLPQLSDLGLLRLCTWLLALSPDLSLSNATVLTRSLFLGRILSLTSSASRLLTTALTSFCAKYTYPVCSALLDPVLQAPGTGPAQTELLCCLVKMESLEPDAQVLMLGQILELPWKEETFLVLQSLLERQVEMTPEKFSVLMEKLCKKGLAATTSMAYAKLMLTVMTKYQANITETQRLGLAMALEPNTTFLRKSLKAALKHLGP

SEQ ID NO: 39 - protein, 374 aa, Homo sapiens
MESLLQHLDRFSELLAVSSTTYVSTWDPATVRRALQWARYLRHIHRRFGRHGPIRTALERRLHNQWRQEGGFGRGPVPGLANFQALGHCDVLLSLRLLENRALGDAARYHLVQQLFPGPGVRDADEETLQESLARLARRRSAVHMLRFNGYRENPNLQEDSLMKTQAELLLERLQEVGKAEAERPARFLSSLWERLPQNNFLKVIAVALLQPPLSRRPQEELEPGIHKSPGEGSQVLVHWLLGNSEVFAAFCRALPAGLLTLVTSRHPALSPVYLGLLTDWGQRLHYDLQKGIWVGTESQDVPWEELHNRFQSLCQAPPPLKDKVLTALETCKAQDGDFEVPGLSIWTDLLLALRSGAFRKRQVLGLSAGLSSV

SEQ ID NO: 40 - protein, 622 aa, Homo sapiens
MSRQTTSVGSSCLDLWREKNDRLVRQAKVAQNSGLTLRRQQLAQDALEGLRGLLHSLQGLPAAVPVLPLELTVTCNFIILRASLAQGFTEDQAQDIQRSLERVLETQEQQGPRLEQGLRELWDSVLRASCLLPELLSALHRLVGLQAALWLSADRLGDLALLLETLNGSQSGASKDLLLLLKTWSPPAEELDAPLTLQDAQGLKDVLLTAFAYRQGLQELITGNPDKALSSLHEAASGLCPRPVLVQVYTALGSCHRKMGNPQRALLYLVAALKEGSAWGPPLLEASRLYQQLGDTTAELESLELLVEALNVPCSSKAPQFLIEVELLLPPPDLASPLHCGTQSQTKHILASRCLQTGRAGDAAEHYLDLLALLLDSSEPRFSPPPSPPGPCMPEVFLEAAVALIQAGRAQDALTLCEELLSRTSSLLPKMSRLWEDARKGTKELPYCPLWVSATHLLQGQAWVQLGAQKVAISEFSRCLELLFRATPEEKEQGAAFNCEQGCKSDAALQQLRAAALISRGLEWVASGQDTKALQDFLLSVQMCPGNRDTYFHLLQTLKRLDRRDEATALWWRLEAQTKGSHEDALWSLPLYLESYLSWIRPSDRDAFLEEFRTSLPKSCDL

SEQ ID NO: 41 - DNA, 1110 bp, Artificial sequence
  codon optimized human gammaC DNA
atgctgaaaccaagcctgccctttacaagcctgctgttcctgcagctgccactgctgggggtcggactgaatactacaatcctgacaccaaacggaaatgaggacaccacagccgatttctttctgactaccatgcccactgacagtctgtcagtgagcaccctgccactgcccgaggtccagtgcttcgtgtttaacgtcgaatatatgaactgtacctggaatagctcctctgaacctcagccaacaaatctgactctgcactactggtataagaactctgacaatgataaggtgcagaaatgctcacattatctgttcagcgaggaaatcacctccggctgtcagctgcagaagaaagagattcacctgtaccagacatttgtggtccagctgcaggatccccgggaacctcggagacaggccactcagatgctgaagctgcagaacctggtcatcccatgggctcccgagaatctgaccctgcataaactgtccgagtctcagctggaactgaactggaacaataggttcctgaatcactgcctggagcatctggtgcagtaccgcacagactgggatcactcttggactgaacagagtgtggactatcgacataagtttagtctgccttcagtggatgggcagaaaaggtacacattcagggtccgctctcggttcaacccactgtgcggaagcgcccagcactggagcgagtggtcccaccccatccattgggggtctaacaccagcaaggagaatcctttcctgtttgccctggaagctgtggtcatttcagtgggaagcatgggcctgatcattagcctgctgtgcgtgtacttctggctggagcggaccatgcctagaatcccaacactgaagaacctggaggacctggtgacagaatatcacggcaatttttccgcttggtctggggtcagtaaaggactggcagagagcctgcagcccgattactccgagcggctgtgcctggtgtccgaaattccccctaaaggcggggcactgggagaaggccctggggcctccccctgcaaccagcactcaccctattgggcaccaccctgttacaccctgaaacccgaaacttaa

SEQ ID NO: 42 - DNA, 1110 bp, Homo sapiens
atgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagcgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctga

SEQ ID NO: 43 - DNA, 1114 bp, Canis lupus familiaris
atgttgaagccaccattgccactcagatccctcttattcctgcagctgtctctgctgggggtggggctgaactccacggtccccatgcccaatgggaatgaagacatcacacctgatttcttcctgaccgctacaccctccgagaccctcagtgtttcctccctgcccctcccagaggtccagtgttttgtgttcaatgttgagtacatgaattgcacttggaacagcagctctgagccccggcccaccaacctgaccctgcactactggtataagaactccaatgatgataaagtccaggagtgtggccactacctattctctagagaggtcactgctggctgttggttgcagaaggaggagatccatctctacgaaacatttgttgtccagctccgggacccacgggaacccaggaggcagtccacacagaagctaaaactgcaaaatctggtgatcccctgggctccggagaacctaacccttcacaacctgagcgaatcccagctagaactgagctggagcaacagacacttggaccactgtttggagcatgttgtgcagtaccggagtgactgggaccgcagctggactgaacagtcagtggaccaccgaaatagcttctctctgcctagcgtggatgggcagaagttctacacgttccgtgtccgaagccgctataacccactctgtggaagcgctcagcgttggagtgaatggagccaccctatccactgggggagcaatacctccaaggagaatcctttgtttgcatcggaagctgtgcttatcccccttggctccatgggattgattattagccttatctgtgtgtactactggctggaacggtcgatcccccgaattcctaccctcaagaacctggaggatctggttactgaatatcacgggaatttttcggcctggagtggagtgtctaagggactggcggagagtctgcagccagactacagtgaatggctctgccacgtcagtgagattcccccaaaaggaggggctccaggggagggtcctgggggctccccctgcagccagcatagcccctactgggctcccccatgttataccctgaaacctgaaactggagccctga

SEQ ID NO: 44 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 45 - protein, 373 aa, Canis lupus familiaris
MLKPPLPLRSLLFLQLSLLGVGLNSTVPMPNGNEDITPDFFLTATPSETLSVSSLPLPEVQCFVFNVEYMNCTWNSSSEPRPTNLTLHYWYKNSNDDKVQECGHYLFSREVTAGCWLQKEEIHLYETFVVQLRDPREPRRQSTQKLKLQNLVIPWAPENLTLHNLSESQLELSWSNRHLDHCLEHVVQYRSDWDRSWTEQSVDHRNSFSLPSVDGQKFYTFRVRSRYNPLCGSAQRWSEWSHPIHWGSNTSKENPLFASEAVLIPLGSMGLIISLICVYYWLERSIPRIPTLKNLEDLVTEYHGNFSAWSGVSKGLAESLQPDYSEWLCHVSEIPPKGGAPGEGPGGSPCSQHSPYWAPPCYTLKPETGALIP

SEQ ID NO: 46 - DNA, 3375 bp, Homo sapiens
atggcacctccaagtgaagagacgcccctgatccctcagcgttcatgcagcctcttgtccacggaggctggtgccctgcatgtgctgctgcccgctcggggccccgggcccccccagcgcctatctttctcctttggggaccacttggctgaggacctgtgcgtgcaggctgccaaggccagcggcatcctgcctgtgtaccactccctctttgctctggccacggaggacctgtcctgctggttccccccgagccacatcttctccgtggaggatgccagcacccaagtcctgctgtacaggattcgcttttacttccccaattggtttgggctggagaagtgccaccgcttcgggctacgcaaggatttggccagtgctatccttgacctgccagtcctggagcacctctttgcccagcaccgcagtgacctggtgagtgggcgcctccccgtgggcctcagtctcaaggagcagggtgagtgtctcagcctggccgtgttggacctggcccggatggcgcgagagcaggcccagcggccgggagagctgctgaagactgtcagctacaaggcctgcctacccccaagcctgcgcgacctgatccagggcctgagcttcgtgacgcggaggcgtattcggaggacggtgcgcagagccctgcgccgcgtggccgcctgccaggcagaccggcactcgctcatggccaagtacatcatggacctggagcggctggatccagccggggccgccgagaccttccacgtgggcctccctggggcccttggtggccacgacgggctggggctgctccgcgtggctggtgacggcggcatcgcctggacccagggagaacaggaggtcctccagcccttctgcgactttccagaaatcgtagacattagcatcaagcaggccccgcgcgttggcccggccggagagcaccgcctggtcactgttaccaggacagacaaccagattttagaggccgagttcccagggctgcccgaggctctgtcgttcgtggcgctcgtggacggctacttccggctgaccacggactcccagcacttcttctgcaaggaggtggcaccgccgaggctgctggaggaagtggccgagcagtgccacggccccatcactctggactttgccatcaacaagctcaagactgggggctcacgtcctggctcctatgttctccgccgcagcccccaggactttgacagcttcctcctcactgtctgtgtccagaacccccttggtcctgattataagggctgcctcatccggcgcagccccacaggaaccttccttctggttggcctcagccgaccccacagcagtcttcgagagctcctggcaacctgctgggatggggggctgcacgtagatggggtggcagtgaccctcacttcctgctgtatccccagacccaaagaaaagtccaacctgatcgtggtccagagaggtcacagcccacccacatcatccttggttcagccccaatcccaataccagctgagtcagatgacatttcacaagatccctgctgacagcctggagtggcatgagaacctgggccatgggtccttcaccaagatttaccggggctgtcgccatgaggtggtggatggggaggcccgaaagacagaggtgctgctgaaggtcatggatgccaagcacaagaactgcatggagtcattcctggaagcagcgagcttgatgagccaagtgtcgtaccggcatctcgtgctgctccacggcgtgtgcatggctggagacagcaccatggtgcaggaatttgtacacctgggggccatagacatgtatctgcgaaaacgtggccacctggtgccagccagctggaagctgcaggtggtcaaacagctggcctacgccctcaactatctggaggacaaaggcctgccccatggcaatgtctctgcccggaaggtgctcctggctcgggagggggctgatgggagcccgcccttcatcaagctgagtgaccctggggtcagccccgctgtgttaagcctggagatgctcaccgacaggatcccctgggtggcccccgagtgtctccgggaggcgcagacacttagcttggaagctgacaagtggggcttcggcgccacggtctgggaagtgtttagtggcgtcaccatgcccatcagtgccctggatcctgctaagaaactccaattttatgaggaccggcagcagctgccggcccccaagtggacagagctggccctgctgattcaacagtgcatggcctatgagccggtccagaggccctccttccgagccgtcattcgtgacctcaatagcctcatctcttcagactatgagctcctctcagaccccacacctggtgccctggcacctcgtgatgggctgtggaatggtgcccagctctatgcctgccaagaccccacgatcttcgaggagagacacctcaagtacatctcacagctgggcaagggcaactttggcagcgtggagctgtgccgctatgacccgctaggcgacaatacaggtgccctggtggccgtgaaacagctgcagcacagcgggccagaccagcagagggactttcagcgggagattcagatcctcaaagcactgcacagtgatttcattgtcaagtatcgtggtgtcagctatggcccgggccgccagagcctgcggctggtcatggagtacctgcccagcggctgcttgcgcgacttcctgcagcggcaccgcgcgcgcctcgatgccagccgcctccttctctattcctcgcagatctgcaagggcatggagtacctgggctcccgccgctgcgtgcaccgcgacctggccgcccgaaacatcctcgtggagagcgaggcacacgtcaagatcgctgacttcggcctagctaagctgctgccgcttgacaaagactactacgtggtccgcgagccaggccagagccccattttctggtatgcccccgaatccctctcggacaacatcttctctcgccagtcagacgtctggagcttcggggtcgtcctgtacgagctcttcacctactgcgacaaaagctgcagcccctcggccgagttcctgcggatgatgggatgtgagcgggatgtccccgccctctgccgcctcttggaactgctggaggagggccagaggctgccggcgcctcctgcctgccctgctgaggttcacgagctcatgaagctgtgctgggcccctagcccacaggaccggccatcattcagcgccctgggcccccagctggacatgctgtggagcggaagccgggggtgtgagactcatgccttcactgctcacccagagggcaaacaccactccctgtccttttcatag

SEQ ID NO: 47 - DNA, 870 bp, Homo sapiens
atggagaacggatacacctatgaagattataagaacactgcagaatggcttctgtctcacactaagcaccgacctcaagttgcaataatctgtggttctggattaggaggtctgactgataaattaactcaggcccagatctttgactacggtgaaatccccaactttccccgaagtacagtgccaggtcatgctggccgactggtgtttgggttcctgaatggcagggcctgtgtgatgatgcagggcaggttccacatgtatgaagggtacccactctggaaggtgacattcccagtgagggttttccaccttctgggtgtggacaccctggtagtcaccaatgcagcaggagggctgaaccccaagtttgaggttggagatatcatgctgatccgtgaccatatcaacctacctggtttcagtggtcagaaccctctcagagggcccaatgatgaaaggtttggagatcgtttccctgccatgtctgatgcctacgaccggactatgaggcagagggctctcagtacctggaaacaaatgggggagcaacgtgagctacaggaaggcacctatgtgatggtggcaggccccagctttgagactgtggcagaatgtcgtgtgctgcagaagctgggagcagacgctgttggcatgagtacagtaccagaagttatcgttgcacggcactgtggacttcgagtctttggcttctcactcatcactaacaaggtcatcatggattatgaaagcctggagaaggccaaccatgaagaagtcttagcagctggcaaacaagctgcacagaaattggaacagtttgtctccattcttatggccagcattccactccctgacaaagccagttga

SEQ ID NO: 48 - DNA, 1092 bp, Homo sapiens
atggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtagccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctga

SEQ ID NO: 49 - DNA, 3132 bp, Homo sapiens
atggcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggccggccccgccaacatcttctgtcgctgactcggagagctcagaagcaccggctgagggagctcaagctgcaagtcaaagcctttgctgacaaagaagaaggtggagatgtgaagtccgtgtgcatgaccttgttcctgctggctctgagggcgaggaatgagcacaggcaagctgatgagctggaggccatcatgcagggaaagggctctggcctgcagccagctgtttgcttggccatccgtgtcaacaccttcctcagctgcagtcagtaccacaagatgtacaggactgtgaaagccatcacagggagacagatttttcagcctttgcatgcccttcggaatgctgagaaggtacttctgccaggctaccaccactttgagtggcagccacctctgaagaatgtgtcttccagcactgatgttggcattattgatgggctgtctggactatcatcctctgtggatgattacccagtggacaccattgcaaagaggttccgctatgattcagctttggtgtctgctttgatggacatggaagaagacatcttggaaggcatgagatcccaagaccttgatgattacctgaatggccccttcactgtggtggtgaaggagtcttgtgatggaatgggagacgtgagtgagaagcatgggagtgggcctgtagttccagaaaaggcagtccgtttttcattcacaatcatgaaaattactattgcccacagctctcagaatgtgaaagtatttgaagaagccaaacctaactctgaactgtgttgcaagccattgtgccttatgctggcagatgagtctgaccacgagacgctgactgccatcctgagtcctctcattgctgagagggaggccatgaagagcagtgaattaatgcttgagctgggaggcattctccggactttcaagttcatcttcaggggcaccggctatgatgaaaaacttgtgcgggaagtggaaggcctcgaggcttctggctcagtctacatttgtactctttgtgatgccacccgtctggaagcctctcaaaatcttgtcttccactctataaccagaagccatgctgagaacctggaacgttatgaggtctggcgttccaacccttaccatgagtctgtggaagaactgcgggatcgggtgaaaggggtctcagctaaacctttcattgagacagtcccttccatagatgcactccactgtgacattggcaatgcagctgagttctacaagatcttccagctagagataggggaagtgtataagaatcccaatgcttccaaagaggaaaggaaaaggtggcaggccacactggacaagcatctccggaagaagatgaacctcaaaccaatcatgaggatgaatggcaactttgccaggaagctcatgaccaaagagactgtggatgcagtttgtgagttaattccttccgaggagaggcacgaggctctgagggagctgatggatctttacctgaagatgaaaccagtatggcgatcatcatgccctgctaaagagtgcccagaatccctctgccagtacagtttcaattcacagcgttttgctgagctcctttctacgaagttcaagtataggtatgagggaaaaatcaccaattattttcacaaaaccctggcccatgttcctgaaattattgagagggatggctccattggggcatgggcaagtgagggaaatgagtctggtaacaaactgtttaggcgcttccggaaaatgaatgccaggcagtccaaatgctatgagatggaagatgtcctgaaacaccactggttgtacacctccaaatacctccagaagtttatgaatgctcataatgcattaaaaacctctgggtttaccatgaaccctcaggcaagcttaggggacccattaggcatagaggactctctggaaagccaagattcaatggaattttaa

SEQ ID NO: 50 - DNA, 1584 bp, Homo sapiens
atgtctctgcagatggtaacagtcagtaataacatagccttaattcagccaggcttctcactgatgaattttgatggacaagttttcttctttggacaaaaaggctggcccaaaagatcctgccccactggagttttccatctggatgtaaagcataaccatgtcaaactgaagcctacaattttctctaaggattcctgctacctccctcctcttcgctacccagccacttgcacattcaaaggcagcttggagtctgaaaagcatcaatacatcatccatggagggaaaacaccaaacaatgaggtttcagataagatttatgtcatgtctattgtttgcaagaacaacaaaaaggttacttttcgctgcacagagaaagacttggtaggagatgttcctgaagccagatatggtcattccattaatgtggtgtacagccgagggaaaagtatgggtgttctctttggaggacgctcatacatgccttctacccacagaaccacagaaaaatggaatagtgtagctgactgcctgccctgtgttttcctggtggattttgaatttgggtgtgctacatcatacattcttccagaacttcaggatgggctatcttttcatgtctctattgccaaaaatgacaccatctatattttaggaggacattcacttgccaataatatccggcctgccaacctgtacagaataagggttgatcttcccctgggtagcccagctgtgaattgcacagtcttgccaggaggaatctctgtctccagtgcaatcctgactcaaactaacaatgatgaatttgttattgttggtggctatcagcttgaaaatcaaaaaagaatgatctgcaacatcatctctttagaggacaacaagatagaaattcgtgagatggagaccccagattggaccccagacattaagcacagcaagatatggtttggaagcaacatgggaaatggaactgtttttcttggcataccaggagacaataaacaagttgtttcagaaggattctatttctatatgttgaaatgtgctgaagatgatactaatgaagagcagacaacattcacaaacagtcaaacatcaacagaagatccaggggattccactccctttgaagactctgaagaattttgtttcagtgcagaagcaaatagttttgatggtgatgatgaatttgacacctataatgaagatgatgaagaagatgagtctgagacaggctactggattacatgctgccctacttgtgatgtggatatcaacacttgggtaccattctattcaactgagctcaacaaacccgccatgatctactgctctcatggggatgggcactgggtccatgctcagtgcatggatctggcagaacgcacactcatccatctgtcagcaggaagcaacaagtattactgcaatgagcatgtggagatagcaagagctctacacactccccaaagagtcctacccttaaaaaagcctccaatgaaatccctccgtaaaaaaggttctggaaaaatcttgactcctgccaagaaatcctttcttagaaggttgtttgattag

SEQ ID NO: 51 - protein, 1124 aa, Homo sapiens
MAPPSEETPLIPQRSCSLLSTEAGALHVLLPARGPGPPQRLSFSFGDHLAEDLCVQAAKASGILPVYHSLFALATEDLSCWFPPSHIFSVEDASTQVLLYRIRFYFPNWFGLEKCHRFGLRKDLASAILDLPVLEHLFAQHRSDLVSGRLPVGLSLKEQGECLSLAVLDLARMAREQAQRPGELLKTVSYKACLPPSLRDLIQGLSFVTRRRIRRTVRRALRRVAACQADRHSLMAKYIMDLERLDPAGAAETFHVGLPGALGGHDGLGLLRVAGDGGIAWTQGEQEVLQPFCDFPEIVDISIKQAPRVGPAGEHRLVTVTRTDNQILEAEFPGLPEALSFVALVDGYFRLTTDSQHFFCKEVAPPRLLEEVAEQCHGPITLDFAINKLKTGGSRPGSYVLRRSPQDFDSFLLTVCVQNPLGPDYKGCLIRRSPTGTFLLVGLSRPHSSLRELLATCWDGGLHVDGVAVTLTSCCIPRPKEKSNLIVVQRGHSPPTSSLVQPQSQYQLSQMTFHKIPADSLEWHENLGHGSFTKIYRGCRHEVVDGEARKTEVLLKVMDAKHKNCMESFLEAASLMSQVSYRHLVLLHGVCMAGDSTMVQEFVHLGAIDMYLRKRGHLVPASWKLQVVKQLAYALNYLEDKGLPHGNVSARKVLLAREGADGSPPFIKLSDPGVSPAVLSLEMLTDRIPWVAPECLREAQTLSLEADKWGFGATVWEVFSGVTMPISALDPAKKLQFYEDRQQLPAPKWTELALLIQQCMAYEPVQRPSFRAVIRDLNSLISSDYELLSDPTPGALAPRDGLWNGAQLYACQDPTIFEERHLKYISQLGKGNFGSVELCRYDPLGDNTGALVAVKQLQHSGPDQQRDFQREIQILKALHSDFIVKYRGVSYGPGRQSLRLVMEYLPSGCLRDFLQRHRARLDASRLLLYSSQICKGMEYLGSRRCVHRDLAARNILVESEAHVKIADFGLAKLLPLDKDYYVVREPGQSPIFWYAPESLSDNIFSRQSDVWSFGVVLYELFTYCDKSCSPSAEFLRMMGCERDVPALCRLLELLEEGQRLPAPPACPAEVHELMKLCWAPSPQDRPSFSALGPQLDMLWSGSRGCETHAFTAHPEGKHHSLSFS

SEQ ID NO: 52 - protein, 289 aa, Homo sapiens
MENGYTYEDYKNTAEWLLSHTKHRPQVAIICGSGLGGLTDKLTQAQIFDYGEIPNFPRSTVPGHAGRLVFGFLNGRACVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAECRVLQKLGADAVGMSTVPEVIVARHCGLRVFGFSLITNKVIMDYESLEKANHEEVLAAGKQAAQKLEQFVSILMASIPLPDKAS

SEQ ID NO: 53 - protein, 363 aa, Homo sapiens
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 54 - protein, 1043 aa, Homo sapiens
MAASFPPTLGLSSAPDEIQHPHIKFSEWKFKLFRVRSFEKTPEEAQKEKKDSFEGKPSLEQSPAVLDKADGQKPVPTQPLLKAHPKFSKKFHDNEKARGKAIHQANLRHLCRICGNSFRADEHNRRYPVHGPVDGKTLGLLRKKEKRATSWPDLIAKVFRIDVKADVDSIHPTEFCHNCWSIMHRKFSSAPCEVYFPRNVTMEWHPHTPSCDICNTARRGLKRKSLQPNLQLSKKLKTVLDQARQARQHKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDPLGIEDSLESQDSMEF

SEQ ID NO: 55 - protein, 527 aa, Homo sapiens
MSLQMVTVSNNIALIQPGFSLMNFDGQVFFFGQKGWPKRSCPTGVFHLDVKHNHVKLKPTIFSKDSCYLPPLRYPATCTFKGSLESEKHQYIIHGGKTPNNEVSDKIYVMSIVCKNNKKVTFRCTEKDLVGDVPEARYGHSINVVYSRGKSMGVLFGGRSYMPSTHRTTEKWNSVADCLPCVFLVDFEFGCATSYILPELQDGLSFHVSIAKNDTIYILGGHSLANNIRPANLYRIRVDLPLGSPAVNCTVLPGGISVSSAILTQTNNDEFVIVGGYQLENQKRMICNIISLEDNKIEIREMETPDWTPDIKHSKIWFGSNMGNGTVFLGIPGDNKQVVSEGFYFYMLKCAEDDTNEEQTTFTNSQTSTEDPGDSTPFEDSEEFCFSAEANSFDGDDEFDTYNEDDEEDESETGYWITCCPTCDVDINTWVPFYSTELNKPAMIYCSHGDGHWVHAQCMDLAERTLIHLSAGSNKYYCNEHVEIARALHTPQRVLPLKKPPMKSLRKKGSGKILTPAKKSFLRRLFD

SEQ ID NO: 56 - DNA, 511 bp, Homo sapiens
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 57 - DNA, 22 bp, Artificial sequence
  forward primer for lentivirus backbone
agagatgggtgcgagagcgtca

SEQ ID NO: 58 - DNA, 24 bp, Artificial sequence
  reverse primer for lentivirus backbone
tgccttggtgggtgctactcctaa

SEQ ID NO: 59 - DNA, 21 bp, Artificial sequence
  forward primer for beta-actin
tcctgtggcatcgacgaaact

SEQ ID NO: 60 - DNA, 21 bp, Artificial sequence
  reverse primer for beta-actin
gaagcatttgcggtggacgat

SEQ ID NO: 61 - DNA, 20 bp, Artificial sequence
  lentivirus-specific forward primer for PCR
tgaaagcgaaagggaaacca

SEQ ID NO: 62 - DNA, 15 bp, Artificial sequence
  lentivirus-specific reverse primer for PCR
ccgtgcgcgcttcag

SEQ ID NO: 63 - DNA, 24 bp, Artificial sequence
  lentivirus-specific probe for PCR
agctctctcgacgcaggactcggc

SEQ ID NO: 64 - DNA, 22 bp, Artificial sequence
  forward beta-globin primer for PCR
cctatcagaaagtggtggctgg

SEQ ID NO: 65 - DNA, 23 bp, Artificial sequence
  reverse beta-globin primer for PCR
ttggacagcaagaaagtgagctt

SEQ ID NO: 66 - DNA, 25 bp, Artificial sequence
  beta-globin probe for PCR
tggctaatgccctggcccacaagta

(19) United States
(12) Patent Application Publication
(10) Pub. No.: US 2020/0199534 A1
(43) Pub. Date: Jun. 25, 2020

(54) THERAPEUTIC FORMULATIONS CONTAINING CD34+ STEM CELLS DERIVED FROM NEGATIVE SELECTION

(71) Applicant: Fred Hutchinson Cancer Research Center, Seattle, WA (US)

(72) Inventors: Jennifer E. Adair, Seattle, WA (US); Hans-Peter Kiem, Seattle, WA (US)

(73) Assignee: Fred Hutchinson Cancer Research Center, Seattle, WA (US)

(21) Appl. No.: 16/608,778

(22) PCT Filed: Apr. 27, 2018
(86) PCT No.: PCT/US2018/029983

§ 371 Date: Oct. 25, 2019

Related U.S. Application Data
(60) Provisional application No. 62/491,116, filed on Apr. 27, 2017.
(60) Provisional application No. 62/503,801, filed on May 9, 2017.

(51) Int. Cl. C12N 5/0789

(52) U.S. Cl. CPC C12N 5/0647; C12N 2501/515; C12N 2740/16011; C12N 2501/599; C12N 2510/00

(57) ABSTRACT

Therapeutic formulations containing CD34+ stem cells derived from negative selection are described. The cells within the formulations can be genetically-modified for a number of therapeutic purposes. The disclosure is particularly useful for the treatment of patients with fragile stem cells or stem cells with low CD34+ expression levels.

### CROSS-REFERENCE TO RELATED APPLICATION

This application claims priority to U.S. Provisional Patent Application No. 62/491,116 filed on Apr. 27, 2017, and to U.S. Provisional Patent Application No. 62/503,801 filed May 9, 2017, both of which are incorporated herein by reference in their entirety as if fully set forth herein.

### REFERENCE TO SEQUENCE LISTING

A computer readable text file, entitled “17-098-WO-PCT_ST25.txt” created on Apr. 24, 2018, with a file size of 176 KB, contains the sequence listing for this application and is hereby incorporated by reference in its entirety.

### FIELD OF THE DISCLOSURE

The current disclosure provides therapeutic formulations containing CD34+ stem cells derived from negative selection. The cells within the formulations can be genetically-modified for a number of therapeutic purposes. The disclosure is particularly useful for the treatment of patients with fragile stem cells or stem cells with low CD34+ expression levels.

### BACKGROUND OF THE DISCLOSURE

Hematopoietic stem cells (HSC) are stem cells that can give rise to all blood cell types such as the white blood cells of the immune system (e.g., virus-fighting T cells and antibody-producing B cells) and red blood cells. The therapeutic administration of HSC can be used to treat a variety of adverse conditions including immune deficiency diseases, blood disorders, malignant cancers, infections, and radiation exposure (e.g., cancer treatment, accidental, or attack-based). As examples, more than 80 primary immune deficiency diseases are recognized by the World Health Organization. These diseases are characterized by an intrinsic defect in the immune system in which, in some cases, the body is unable to produce any or enough antibodies against infection. In other cases, cellular defenses to fight infection fail to work properly. Typically, primary immune deficiencies are inherited disorders.

One example of a primary immune deficiency is Fanconi anemia (FA). FA is an inherited blood disorder that leads to bone marrow (BM) failure. It is characterized, in part, by a deficient DNA-repair mechanism. At least 20% of patients with FA develop cancers such as acute myeloid leukemias, and cancers of the skin, liver, gastrointestinal tract, and gynecological system. The skin and gastrointestinal tumors are usually squamous cell carcinomas. The average age of patients who develop cancer is 15 years for leukemia, 16 years for liver tumors, and 23 years for other tumors.

Another example of a primary immune deficiency is severe combined immunodeficiency (SCID). SCID is a genetic disease that results in the absence of a functioning immune system due to the absence of T cells, the absence of natural killer (NK) cells, and the absence of functioning B cells. SCID is often fatal in the first two years of life unless the immune system is reconstituted, for example, through BM transplant (BMT) or gene therapy.

Secondary, or acquired, immune deficiencies are not the result of inherited genetic abnormalities, but rather occur in individuals in which the immune system is compromised by factors outside the immune system. Examples include trauma, viruses, chemotherapy, toxins, and pollution. Acquired immunodeficiency syndrome (AIDS) is an example of a secondary immune deficiency disorder caused by a virus, the human immunodeficiency virus (HIV), in which a depletion of T lymphocytes renders the body unable to fight infection.

In the clinical setting, HSC are obtained for a therapeutic purpose by procuring a biological sample including HSC (e.g., a BM sample) and then manipulating the sample to obtain the HSC out of it. HSC can be “identified” in the sample based on particular proteins that they express on the cell surface.

The current gold standard for obtaining HSC from a biological sample is based on the cell surface protein, CD34. For example, HSC expressing CD34 can be positively selected for using an antibody that binds to CD34. In some procedures the antibody that binds CD34 includes a magnetic element so that the antibody-bound CD34+ HSC can be magnetically separated from the rest of the sample. In other procedures, the antibody can include a fluorescent element so that the antibody-bound CD34+ HSC can be separated from the sample using a fluorescence-activated cell sorter. Other procedures can attach copies of the CD34 binding antibody to a solid matrix (e.g., a plate) and the sample can be passed over the solid matrix so that the CD34+ HSC are “caught” by the antibodies. This procedure is referred to as “panning”.

Each of the above-described procedures to obtain CD34+ HSC from a biological sample uses positive selection for CD34+ HSC. That is, the HSC are identified based on the presence of CD34 (i.e., CD34+) and directly separated from the rest of the sample. Unfortunately, however, such positive laboratory manipulations can damage and/or kill cells. Another drawback is that the antibody bound to the cell, whether attached to a magnetic element or a fluorescent element, remains bound to the therapeutic cells when they are reintroduced into a patient. If the patient has a functioning immune system, the immune system will attack the foreign magnetic element or fluorescent element resulting in unintended inflammatory consequences in the patient.

Based on these drawbacks, among others, another approach that has been considered is to isolate CD34+ HSC using negative selection. In negative selection, the purpose is to identify and remove cells that are not CD34+ HSC from the sample. In this manner, the therapeutic cell of interest is not directly manipulated so that the negative effects of laboratory manipulations are reduced. Thus, one idea has been to replace positive selection of CD34+ HSC with negative selection to remove other cell types.

One impediment to the use of negative selection to obtain a therapeutic population of CD34+ HSC is the diverse amount of cell types that are present in a starting biological sample. Each cell type expresses a distinct array of proteins on the cellular surface, and it is not feasible to use one antibody per cell type to remove all unwanted cells but CD34+ HSC from a sample. Thus, despite the fact that negative selection would involve less direct manipulation of CD34+ HSC, positive selection has remained the clinical standard to use.

While continuing to use positive selection of CD34+ HSC in the clinic, additional drawbacks to this approach have emerged, particularly in relation to individual diseases or conditions to be treated. For example, a hallmark of FA is accelerated decline in CD34+ HSC leading to BM failure. Due to the relatively limited CD34+ HSC cell population in FA patients, it is difficult to obtain samples with enough starting CD34+ HSC. Because of the low starting numbers, it becomes even more paramount that existing CD34+ HSC in the sample not be harmed during processing and formulation. Further, in working with FA patients, additional drawbacks described further herein made it even more apparent that alternatives to positive CD34+ HSC selection were needed.

### SUMMARY OF THE DISCLOSURE

The current disclosure provides systems and methods to achieve negative selection of CD34+ HSC. The systems and methods address numerous challenges of the prior art that continued to lead to the previous reliance on positive CD34+ HSC selection. They also address new, previously unknown challenges in the collection of CD34+ HSC in certain disease states. For example, in addition to having low absolute numbers of CD34+ HSC cells, the current disclosure provides that many of the existing CD34+ HSC cells in FA patients express a relatively low amount of CD34. A low expression level of CD34 means that fewer cells will be identified during positive selection due to a weak CD34 signal. Even if obtained, the current disclosure provides that it is primarily the CD34+ HSC expressing high amounts of CD34 that can effectively be used for certain therapeutic purposes. Moreover, the fraction of CD34+ HSC that can be derived from FA patients for a therapeutic purpose are exceedingly fragile and susceptible to damage due to positive selection laboratory processing. Accordingly, due to the very low number of HSC expressing optimal CD34+ levels in certain patient populations and their fragility, it becomes exceedingly important to not endanger these existing cells with the laboratory manipulations that positive selection imposes.

The systems and methods disclosed herein achieve therapeutic formulations containing CD34+ HSC derived from negative selection by: (1) identifying and removing cells from a biological sample that are problematic to the development of a therapeutic formulation containing CD34+ HSC; (2) using a selected combination of cell surface markers that efficiently remove these unwanted cells from a sample; and (3) maintaining cells that support the health and maintenance of CD34+ HSC in the laboratory setting.

Regarding advantage (1), “identifying and removing cells from a biological sample that are problematic to the development of a therapeutic formulation containing CD34+ HSC”, there are numerous cell types that are detrimental to both formulating CD34+ HSC for administration and later for administering the formulated HSC to a patient. For example, granulocytes are a short-lived cell type. These cells often die during the time period required for sample processing and formulation, releasing their contents into the therapeutic formulation. When the therapeutic formulation is then administered to a patient, the granulocyte's released cell contents can trigger inflammatory reactions within the patient.

T cells and NK cells can be problematic during sample processing and formulation because they are cytotoxic to other cells. That is, they may kill other beneficial cells, such as therapeutic CD34+ HSC, before the CD34+ HSC can be administered to a patient. This is especially problematic in biological samples that begin with a low number and/or fragile CD34+ HSC.

Other cell types are important to remove because they become detrimental after administration to a subject. For example, monocytes are antigen presenting cells. If they are retained in a formulation and administered to a patient, unwanted immune responses and inflammation will be triggered in the patient.

Regarding advantage (2), “using a selected combination of cell surface markers that efficiently remove unwanted cells from a sample”, each cell type within a biological sample expresses a distinct array of proteins on the cellular surface, and it is not feasible to use one antibody or binding protein per cell type to remove all unwanted cells from a sample. The current disclosure provides combinations of markers that can be used to efficiently target and remove cells to yield therapeutic formulations containing CD34+ HSC derived from negative selection. In particular embodiments, cells expressing CD45RA are targeted and removed from a sample to yield a therapeutic population containing CD34+ HSC obtained through negative selection. In particular embodiments, cells expressing CD45RA, CD3, CD14, CD16, and/or CD19 are targeted and removed from a sample to yield a therapeutic population containing CD34+ HSC obtained through negative selection. In particular embodiments, negative selection results in removal of at least 70%, at least 80%, or at least 90% of the targeted unwanted cells.

Regarding advantage (3), “maintaining cells that support the health and maintenance of CD34+ HSC in the laboratory setting”, the combinations of markers to remove unwanted cells were also selected based on the ability to retain other cells within the therapeutic formulations containing CD34+ HSC. For example, mesenchymal stem cells (MSC), facilitate the health and function of CD34+ HSC during processing and after administration. Thus, in particular embodiments, the combinations of selected markers do not remove MSC.

The disclosed systems and methods provide a clinically-viable alternative to positive CD34+ HSC selection to overcome barriers in isolation of blood stem and progenitor cells which have plagued clinical trials of gene therapies, such as those for FA. The disclosed approaches to preserve available CD34+ HSC during initial blood product processing improves autologous stem cell transplantation, gene therapy, and gene editing, particularly in settings of limited CD34+ HSC availability, including FA and other diseases wherein direct CD34 selection has proven inefficient, such as SCID, sickle cell disease (SCD), and Dyskeratosis congenita. In particular embodiments, use of negative selection preserves at least 50%, at least 70%, or at least 90% of the starting CD34+ HSC population within a sample. In particular embodiments, the CD34+ HSC are CD34+, CD45RA−, and CD90+ HSC.

In particular embodiments, the current disclosure utilizes genetic therapies utilizing viral vectors.

### BRIEF DESCRIPTION OF THE SEVERAL VIEWS OF THE DRAWINGS

Many of the drawings submitted herein are better understood in color. Applicant considers the color versions of the drawings as part of the original submission and reserve the right to present color images of the drawings in later proceedings.

FIG. 1. Clinical characteristics of three patients with Fanconi Anemia A genetic defect enrolled in clinical trial NCT01331018. One adult and two pediatric patients were treated with lentivirus gene therapy for Fanconi Anemia-A (FA-A) defect. Three patients demonstrated steadily declining absolute neutrophils (ANC) and platelet counts in the peripheral blood prior to treatment and less than 30% marrow cellularity. Molecular characterization of the FANCA gene defect performed by gene sequencing demonstrated that Patient 1 was homozygous in the FANCA gene for the splicing variant. For Patient 2, Multiplex Ligation-dependent Probe Amplification (MLBA) on the FANCA gene identified a homozygous gross deletion of exons 6-31. No sequence analysis was performed for Patient 3. *No evaluable marrow with aspiculate particle preparation.

FIGS. 2A-2F. Diminished CD34^{Hi} hematopoietic cells from FA-A patients. CD34 expression in baseline bone marrow ((BM) Patients 1, 2, 3 and healthy donor 1, FIGS. 2A-2D) or mobilized leukapheresis (Patient 3 and healthy donor 2, FIGS. 2E-2F) products was determined by fluorescence staining and flow cytometry analysis. Positive cell fractions are gated based on unstained and isotype stained control samples into two levels of CD34 expression: low expression, CD34^{Lo} or high expression, CD34^{Hi}. The average mean fluorescence intensity (MFI) of CD34^{Lo} population=3512; standard error of the mean (SEM)=627 and CD34^{Hi} population=20070; SEM=5008.

FIGS. 3A-3B. In vitro repopulation potential only restricted to CD34^{Hi} hematopoietic cells. Mobilized leukapheresis from FA-A Patient 3 (FIG. 3A) and a healthy donor (FIG. 3B) were fluorescence stained with anti-CD34 antibody and sort-purified for CD34^{Hi} and low CD34^{Lo} cells. Total nucleated cells (TNC) equivalent to 1,500 CD34-expressing cells were seeded in parallel colony-forming cell (CFC) assays. Percentage of CD34+ cells seeded in the assay that gave rise to colonies is represented as the % of CFC.

FIG. 4. Isolation and LV transduction of autologous hematopoietic FA-A HSPCs.

FIG. 5. Transduction. Viability of the infusion product was determined by trypan blue exclusion dye staining. The vector copy number (VCN) in the bulk transduced population following transduction was determined by quantitative PCR method against a reference standard curve. Plating efficiency of the infusion product was determined as the percentage of CD34+ cells infused with colony-forming capacity. Functional correction of the FANCA gene defect was determined by calculating the plating efficiency under stress of various concentrations of Mitomycin-C. Gene transfer in CFCs was determined as the percentage of colonies analyzed positive for the presence of lentivirus backbone by PCR analysis on DNA extracted from individual colonies.

FIG. 6. Direct CD34 enrichment versus depletion of lineage+ cells. Products can include BM or mAPH (step 1). BM products were first processed through hetastarch sedimentation to deplete red blood cells (RBCs). Leukapheresis products were first subjected to several washes to deplete platelets. For direct CD34+ cell selection, anti-CD34 antibody-bound immunomagnetic beads (microbeads) were used, whereas for lineage depletion anti-CD3+, CD14+, CD16+, and CD19+, microbeads were used (step 2). In both cases, microbead-bound cells are retained on the column and subjected to wash steps. When lineage depletion is used, CD34-expressing cells undergo minimal manipulation during purification. Following purification, cells are cultured and transduced with a VSVG pseudotyped LV at an MOI of 5-10 IU/cell (step 3). Following 16 hours of incubation cells were harvested (step 4). *These processes were performed on the CliniMACS Prodigy™ device from Miltenyi Biotec GmbH, Auburn, Calif.

FIGS. 7A-7B. Flow cytometry analysis and gating strategy of BM and mAPH samples during lineage depletion. Typical profiles of cell samples collected from initial, lineage depleted and lineage enriched products for both BM (FIG. 7A) and mAPH (FIG. 7B). Subsets represented within square brackets ([ ]) above each column represent the parent population for the respective gates. All lineage markers (CD3, CD14, CD15, CD16, CD19, CD20, and CD34) are gated within the CD45+ cell population. The CD45+ cell population in turn is gated within the single cell population identified using forward and side scatter parameters.

FIGS. 8A-8F. Multi-lineage engraftment of lineage depleted and transduced BM and mAPH products in NSG mice. Recovery of TNC, CD34+ cells and lineage+ cells are depicted in FIGS. 8A and 8B. Gene transfer efficiency is represented in FIGS. 8C-8E. The colony-forming potential of transduced cells in standard CFC assays is defined as the plating efficiency (TNC). The colony-forming potential normalized to the number of CD34+ cells seeded is depicted as plating efficiency (CD34+). The percentage of colonies analyzed positive for the presence of lentivirus backbone by PCR analysis on DNA extracted from individual colonies is depicted as transduction efficiency. The vector copy number per cell in the bulk transduced population is depicted as VCN. The average VCN per cell in the individual CFC is depicted as single colony VCN. Data is representative of the average of 9 healthy BM products and 10 healthy mAPH products. Error bars represent the standard error of the mean. (FIG. 8F) Engraftment of human CD45+ cells and lineage development into T cells (CD3+), Monocytes (CD14+) and B cells (CD20+) was determined by flow cytometry over 20 weeks following infusion of lineage depleted cell products. Data is representative of 36 mice from 6 mAPH donors and 42 mice from 6 BM donors respectively. Error bars represent the standard error of the mean.

FIGS. 9A-9D. Representative images of colony morphologies. Lineage depleted and transduced cells from BM or mAPH products were seeded in standard CFC assays. Hematopoietic colonies that arose in the assay were viewed under 4× magnification, wide field and scored for morphology as CFU-macrophage (M) colonies with larger cell appearance and loose association (FIG. 9A), CFU-granulocyte macrophage (GM) with diffuse appearance (FIG. 9B), CFU-erythroid (E) with hemoglobinization and tight association (FIG. 9C) and CFU-granulocyte erythrocyte macrophage megakaryocyte (GEMM) with mixed appearance and partial hemoglobinization (FIG. 9D).

FIGS. 10A-10C. Multi-lineage engraftment levels of lineage depleted cell products in NSG mice is comparable to CD34-enriched cell products from the same donor. (FIG. 10A) Graph depicts percent recovery of total nucleated cells (TNC) and CD34+ cells from each arm following depletion or enrichment. (FIG. 10B) Numbers of total and transduced CFC normalized to 1×10⁸ cells processed to each arm, and VCN in the bulk transduced cells following 10 days of culture. Data is representative of two healthy donor BM products. Error bars represent the standard error of the mean. (FIG. 10C) Engraftment of human CD45+ cells and lineage development into T cells (CD3+), monocytes (CD14+) and B cells (CD20+) was determined by flow cytometry over 26 weeks following infusion. Data is representative of 9 mice for the lineage depleted (Lin−) arm and 6 mice for the CD34 enriched (CD34+) arm respectively.

FIGS. 11A-11C. Blood cell counts for three FA-A patients prior to HSC gene therapy. Graphs depict peripheral blood cell counts in neutrophils (solid black circle), and platelets (solid gray circles) and percent hematocrit (open squares) over time prior to HSC gene therapy intervention for Patient 1 (FIG. 11A), Patient 2 (FIG. 11B), and Patient 3 (FIG. 11C).

FIGS. 12A-12C. Mobilization and Leukapheresis protocol for FA-A Patient 3. Successful mobilization of CD34+ cells was achieved with combination granulocyte-colony stimulating factor (G-CSF; 16 μg/kg BID) and plerixafor (240 μg/kg). FIG. 12A outlines the mobilization drug regimen and the peripheral blood CD34+ cell counts evaluated daily by flow cytometry from day 4 of mobilization. Peripheral blood counts for ANC, platelets and hemoglobin recorded daily through mobilization is depicted in FIG. 12B. FIG. 12C shows patient hematology and intervention following reinfusion.

FIG. 13. Sequences supporting the disclosure.

### DETAILED DESCRIPTION

Hematopoietic stem cells (HSC) are stem cells that can give rise to all blood cell types such as white blood cells and red blood cells. Hematopoietic stem and progenitor cells (HSPC) reflect a slightly more differentiated cell type, but are included within the description of HSC herein. The therapeutic administration of HSC can be used to treat a variety of adverse conditions including immune deficiency diseases, blood disorders, malignant cancers, infections, and radiation exposure (e.g., cancer treatment, accidental, or attack-based). As examples, more than 80 primary immune deficiency diseases are recognized by the World Health Organization. These diseases are characterized by an intrinsic defect in the immune system in which, in some cases, the body is unable to produce any or enough antibodies against infection. In other cases, cellular defenses to fight infection fail to work properly. Typically, primary immune deficiencies are inherited disorders.

One example of a primary immune deficiency is Fanconi anemia (FA). FA is an inherited blood disorder that leads to bone marrow (BM) failure. It is characterized, in part, by a deficient DNA-repair mechanism. At least 20% of patients with FA develop cancers such as acute myeloid leukemias, and cancers of the skin, liver, gastrointestinal tract, and gynecological system. The skin and gastrointestinal tumors are usually squamous cell carcinomas. The average age of patients who develop cancer is 15 years for leukemia, 16 years for liver tumors, and 23 years for other tumors.

Another example of a primary immune deficiency is severe combined immunodeficiency (SCID). SCID is a genetic disease that results in the absence of a functioning immune system due to the absence of T cells, the absence of natural killer (NK) cells, and the absence of functioning B cells. SCID is often fatal in the first two years of life unless the immune system is reconstituted, for example, through BM transplant (BMT) or gene therapy.

Secondary, or acquired, immune deficiencies are not the result of inherited genetic abnormalities, but rather occur in individuals in which the immune system is compromised by factors outside the immune system. Examples include trauma, viruses, chemotherapy, toxins, and pollution. Acquired immunodeficiency syndrome (AIDS) is an example of a secondary immune deficiency disorder caused by a virus, the human immunodeficiency virus (HIV), in which a depletion of T lymphocytes renders the body unable to fight infection.

In the clinical setting, HSC are obtained for a therapeutic purpose by procuring a biological sample including HSC (e.g., a BM sample) and then manipulating the sample to obtain the HSC out of it. HSC can be “identified” in the sample based on particular proteins that they express on their cell surface.

The current gold standard for obtaining HSC from a biological sample is based on the cell surface protein, CD34. For example, HSC expressing CD34 can be positively selected for using an antibody that binds to CD34. In some procedures the antibody includes a magnetic element so that the antibody-bound CD34+ HSC can be magnetically separated from the rest of the sample. In other procedures, the antibody can include a fluorescent element so that the antibody-bound CD34+ HSC can be separated from the sample using a fluorescence-activated cell sorter. Other procedures can attach copies of the CD34 binding antibody to a solid matrix (e.g., a plate) and the sample can be passed over the solid matrix so that the CD34+ HSC are “caught” by the antibodies. This procedure is referred to as “panning”.

Each of the above-described procedures uses positive selection for CD34+ HSC. That is, the HSC are identified based on the presence of CD34 (i.e., CD34+) and directly separated from the rest of the sample. Unfortunately, however, such positive laboratory manipulations can damage and/or kill cells. Another drawback is that the antibody bound to the cell, whether attached to a magnetic element or a fluorescent element remains bound to the therapeutic cells when they are reintroduced into a patient. If the patient has a functioning immune system, the immune system will attack the foreign magnetic element or fluorescent element resulting in unintended inflammatory consequences in the patient.

Based on these drawbacks, among others, another approach that has been considered is to isolate CD34+ HSC using negative selection. In negative selection, the purpose is to identify and remove cells that are not CD34+ HSC from the sample. In this manner, the therapeutic cell of interest is not directly manipulated so that the negative effects of laboratory manipulations are reduced. Thus, one idea has been to replace positive selection of CD34+ HSC with negative selection to remove other cell types.

One impediment to the use of negative selection to obtain a therapeutic population of CD34+ HSC is the diverse amount of cell types that are present in a starting biological sample. Each cell type expresses a distinct array of proteins on the cellular surface, and it is not feasible to use one antibody per cell type to remove all unwanted cells but CD34+ HSC from a sample. Thus, despite the fact that negative selection would involve less direct manipulation of CD34+ HSC, positive selection has remained the clinical standard to use.

While continuing to use positive selection of CD34+ HSC in the clinic, additional drawbacks to this approach have emerged, particularly in relation to individual diseases or conditions to be treated. For example, a hallmark of FA is accelerated decline in CD34+ HSC leading to BM failure. Due to the relatively limited CD34+ HSC cell population in FA patients, it is difficult to obtain samples with enough starting CD34+ HSC. Because of the low starting numbers, it becomes even more paramount that existing CD34+ HSC in the sample not be harmed during processing and formulation. Further, in working with FA patients, additional drawbacks described further in this application made it even more apparent that alternatives to positive CD34+ HSC selection were needed.

The current disclosure provides systems and methods to achieve negative selection of CD34+ HSC. The systems and methods address numerous challenges of the prior art that continued to lead to the previous reliance on positive CD34+ HSC selection. They also address new, previously unknown challenges in the collection of CD34+ HSC in certain disease states. For example, in addition to having low absolute numbers of CD34+ HSC cells, the current disclosure demonstrates an altered ratio of CD34^{Hi} to CD34^{Lo} cells in patients with primary immune deficiencies relative to healthy donors, and an exclusive in vitro repopulating ability in only CD34^{Hi} cells. For example, colony seeding assays demonstrate that only CD34^{Hi} cells contribute to in vitro colony-forming potential in both FA and healthy donor blood products, underscoring the need to preserve as many available CD34+ cells as possible during ex vivo manipulation for gene transfer in these patients.

Importantly, reduction in total CD34+ cell numbers is not restricted to FA. Sickle cell disease (SCD) patients treated with hydroxyurea also display reduced CD34+ cell frequencies in BM and there is a contraindication to mobilization of available CD34+ cells owing to an increased risk for vaso-occlusive crisis. Other inherited BM failure syndromes such as dyskeratosis congenita also are associated with abnormal CD34+ cell frequencies and behavior. As a larger number of disease targets become potentially relevant for gene therapy investigation, likely additional patient populations will display variable CD34+ cell frequency and antigen expression.

Moreover, the fraction of CD34+ HSC that can be derived from FA patients for a therapeutic purpose are exceedingly fragile and susceptible to damage due to positive selection laboratory processing. In particular embodiments, fragile CD34+ cells have increased sensitivity to free radical-induced DNA damage during ex vivo culture and manipulation. In particular embodiments, CD34+ cells are fragile when the cells have less than 20%, less than 15%, less than 10%, less than 5%, less than 1%, or less, colony-forming potential as measured by a standard methylcellulose assay as described herein. In particular embodiments, CD34+ cells are fragile when the cells have no colony-forming potential as measured by a standard methylcellulose assay as described herein. In particular embodiments, CD34+ cells are fragile when there is less than 50%, less than 40%, less than 30%, less than 20%, less than 10%, less than 5%, less than 1%, or less, yield of CD34+ cells of total starting amount of CD34+ cells after positive selection of CD34+ cells for ex vivo culture and manipulation. Accordingly, due to the very low number of these cells in certain patient populations and their fragility, it becomes exceedingly important to not endanger these existing cells with the laboratory manipulations that positive selection imposes.

Particular embodiments described herein provide a clinically viable procedure to indirectly enrich CD34+ cells from a biological sample. In particular embodiments, the processes efficiently deplete >90% of lineage+ cells from healthy donor products while retaining ≥60% of the initial CD34+ cell fraction, reducing total nucleated cells by 1-2 logs, and maintaining transduction efficiency and cell viability following gene transfer. Transduced lineage− cell products engrafted in an immune-deficient mouse model and were equivalent to that of purified CD34+ cells from the same donor when transplanted at matched CD34+ cell doses. This novel selection strategy has been approved by the regulatory agencies in a gene therapy study for patients with FA-A (clinical protocol (NCT01331018)).

The systems and methods achieve therapeutic formulations of CD34+ HSC derived from negative selection by: (1) identifying and removing cells from a biological sample that are problematic to therapeutic CD34+ HSC cell development for various reasons; (2) using a selected combination of cell surface markers that efficiently remove unwanted cells from a sample; and (3) maintaining cells that support the health and maintenance of CD34+ HSC in the laboratory setting.

Regarding advantage (1), “identifying and removing cells from a biological sample that are problematic to therapeutic CD34+ HSC cell development”, there are numerous cell types that are detrimental to both formulating CD34+ HSC for administration and later for administering the formulated HSC to a patient. For example, granulocytes are a short-lived cell type. These cells often die during the time period required for sample processing and formulation, releasing their contents into the therapeutic formulation. When the therapeutic formulation is then administered to a patient, the granulocyte's released cell contents can trigger inflammatory reactions within the patient.

T cells and NK cells can be problematic during sample processing and formulation because they are cytotoxic to other cells. That is, they may kill other beneficial cells, such as therapeutic CD34+ HSC, before the CD34+ HSC can be administered to a patient. This is especially problematic in biological samples that begin with a low number and/or fragile CD34+ HSC.

Other cells are important to remove because they become detrimental after administration to a subject. For example, monocytes are antigen presenting cells. If they are retained in a formulation and administered to a patient, unwanted immune responses and inflammation will be triggered in the patient.

Particular embodiments, include depleting B cells, T cells, monocytes, macrophages, granulocytes, and NK cells (collectively, unwanted cells) from a biological sample. In particular embodiments, negative selection results in at least a 70% reduction in unwanted cells, at least an 80% reduction in unwanted cells, or at least a 90% reduction in unwanted cells. In particular embodiments, “selected unwanted cells” can refer to a particular subset of unwanted cells. In particular embodiments, negative selection results in at least a 70% reduction in CD3+ cells, at least an 80% reduction in CD3+ cells, or at least a 90% reduction in CD3+ cells. In particular embodiments, negative selection results in at least a 70% reduction in CD14+ cells, at least an 80% reduction in CD14+ cells, or at least a 90% reduction in CD14+ cells. In particular embodiments, negative selection results in at least a 70% reduction in CD16+ cells, at least an 80% reduction in CD16+ cells, or at least a 90% reduction in CD16+ cells. In particular embodiments, negative selection results in at least a 70% reduction in CD19+ cells, at least an 80% reduction in CD19+ cells, or at least a 90% reduction in CD19+ cells. In particular embodiments, negative selection results in at least a 70% reduction in CD3+ and CD14+ cells, at least an 80% reduction in CD3+ and CD14+ cells, or at least a 90% reduction in CD3+ and CD14+ cells. In particular embodiments, negative selection results in at least a 70% reduction in CD3+ and CD16+ cells, at least an 80% reduction in CD3+ and CD16+ cells, or at least a 90% reduction in CD3+ and CD16+ cells. In particular embodiments, negative selection results in at least a 70% reduction in CD3+ and CD19+ cells, at least an 80% reduction in CD3+ and CD19+ cells, or at least a 90% reduction in CD3+ and CD19+ cells. In particular embodiments, negative selection results in at least a 70% reduction in CD14+ and CD16+ cells, at least an 80% reduction in CD14+ and CD16+ cells, or at least a 90% reduction in CD14+ and CD16+ cells. In particular embodiments, negative selection results in at least a 70% reduction in CD14+ and CD19+ cells, at least an 80% reduction in CD14+ and CD19+ cells, or at least a 90% reduction in CD14+ and CD19+ cells. In particular embodiments, negative selection results in at least a 70% reduction in CD16+ and CD19+ cells, at least an 80% reduction in CD16+ and CD19+ cells, or at least a 90% reduction in CD16+ and CD19+ cells. In particular embodiments, negative selection results in at least a 70% reduction in CD3+, CD14+ and CD16+ cells, at least an 80% reduction in CD3+, CD14+ and CD16+ cells, or at least a 90% reduction in CD3+, CD14+ and CD16+ cells. In particular embodiments, negative selection results in at least a 70% reduction in CD3+, CD14+ and CD19+ cells, at least an 80% reduction in CD3+, CD14+ and CD19+ cells, or at least a 90% reduction in CD3+, CD14+ and CD19+ cells. In particular embodiments, negative selection results in at least a 70% reduction in CD14+, CD16+ and CD19+ cells, at least an 80% reduction in CD14+, CD16+ and CD19+ cells, or at least a 90% reduction in CD14+, CD16+ and CD19+ cells. In particular embodiments, negative selection results in at least a 70% reduction in CD3+, CD14+, CD16+ and CD19+ cells, at least an 80% reduction in CD3+, CD14+, CD16+ and CD19+ cells, or at least a 90% reduction in CD3+, CD14+, CD16+ and CD19+ cells.

Regarding advantage (2), “using a selected combination of cell surface markers that efficiently remove unwanted cells from a sample”, each cell type within a biological sample expresses a distinct array of proteins on the cellular surface, and it is not feasible to use one antibody per cell type to remove all unwanted cells from a sample. The current disclosure provides combinations of markers that can be used to efficiently target and remove unwanted cells to yield therapeutic formulations of CD34+ HSC derived from negative selection.

In particular embodiments, cells expressing CD45RA are targeted and removed from a sample to yield a therapeutic CD34+ HSC population obtained through negative selection. In particular embodiments, cells expressing CD45RA and CD3 are targeted and removed from a sample to yield a therapeutic CD34+ HSC population obtained through negative selection. In particular embodiments, cells expressing CD45RA and CD14 are targeted and removed from a sample to yield a therapeutic CD34+ HSC population obtained through negative selection. In particular embodiments, cells expressing CD45RA and CD16 are targeted and removed from a sample to yield a therapeutic CD34+ HSC population obtained through negative selection. In particular embodiments, cells expressing CD45RA and CD19 are targeted and removed from a sample to yield a therapeutic CD34+ HSC population obtained through negative selection. In particular embodiments, cells expressing one or more of CD3, CD14, CD16, and/or CD19 are targeted and removed from a sample to yield a therapeutic CD34+ HSC population obtained through negative selection. In particular embodiments, cells expressing CD3, CD14, and CD19 are targeted and removed from a sample to yield a therapeutic CD34+ HSC population obtained through negative selection. In particular embodiments, cells expressing CD3, CD14, CD16, and CD19 are targeted and removed from a sample to yield a therapeutic CD34+ HSC population obtained through negative selection.

Regarding advantage (3), “maintaining cells that support the health and maintenance of CD34+ HSC in the laboratory setting”, the combinations of markers to remove unwanted cells were selected based on the ability to retain other cells within the therapeutic formulations of CD34+ HSC derived from negative selection. In particular embodiments, and especially for BM-derived products, the systems and methods disclosed herein do not include a marker to deplete mesenchymal stem cells (MSC). MSC can facilitate the health and function of CD34+ HSC during processing and after administration. Thus, in particular embodiments, the combinations of selected markers do not remove MSC.

The following sections describe information and steps to support producing therapeutic formulations of CD34+ HSC derived from negative selection: (i) HSC Sources; (ii) Peripheral Blood (PB) Mobilization; (iii) Negative Selection to Yield CD34+ HSC Populations with Accessory Cells; (iv) Genetic Modification of Negatively-Selected CD34+ HSC Populations; (v) Cell Formulation; (vi) Methods of Use; (vii) Reference Levels Derived from Control Populations; and (viii) Kits.

(i) HSC Sources. In particular embodiments, HSC and HSPC are used interchangeably herein. In particular embodiments, HSC are CD34⁺CD45RA⁻CD90⁺ HSC. HSC sources include umbilical cord blood, placental blood, BM and PB (see U.S. Pat. Nos. 5,004,681; 7,399,633; and 7,147,626) Additional sources of HSC include fetal liver, embryonic stem cells (ESC), induced pluripotent stem cells (iPSCs) that can be differentiated into HSCs, aortal-gonadal-mesonephros derived cells, and lymph, liver, thymus, and spleen from age-appropriate donors. Methods regarding collection, anti-coagulation and processing, etc. of blood and tissue samples are well known in the art. All collected sources of HSC can be screened for undesirable components and discarded, treated, or used according to accepted current standards at the time.

When BM is the biological sample, BM can be obtained from the posterior iliac crest by needle aspiration. In particular embodiments, before negative selection, the BM sample can be diluted in N-acetylcysteine (NAC) supplemented cell culture media to obtain a hematocrit value of ≤25%.

(ii) Peripheral Blood (PB) Mobilization. In order to avoid surgical procedures to perform a BM harvest, approaches have been developed to harvest HSC from the PB. Mobilization is a process whereby HSC are stimulated out of the BM niche into the PB for collection. Mobilization can also lead to enhanced proliferation of HSC within the PB. Thus, mobilization allows for a larger frequency of HSC within the PB minimizing the number of days of apheresis to reach a target number for HSC collection and minimizing discomfort to the donor.

In particular embodiments, before obtaining the PB sample, a patient is mobilized until a designated number of CD34+ HSC are detected circulating in the PB. In particular embodiments, the designated number is ≥8 cells/μL, ≥9 cells/μL, or ≥10 cells/μL. In particular embodiments, a patient is mobilized for 5, 6, or 7 days.

In particular embodiments, a patient is mobilized for 6 days. On days 1-6, the patient receives granulocyte-colony stimulating factor (G-CSF; filgrastim) at a dose of 16 μg/kg subcutaneously. On days 4-6, the patient receives the CXCR4 antagonist plerixafor (also known as AMD3100) at a dose of 240 μg/kg subcutaneously.

In particular embodiments, G-CSF can be administered as a single agent followed by concurrent administration with AMD3100. In particular embodiments, a treatment protocol includes a 6 day treatment where G-CSF can be administered on day 1, day 2, day 3, and day 4 and on day 5, G-CSF and AMD3100 are administered 6 to 8 hours prior to PB collection.

In particular embodiments, granulocyte macrophage colony stimulating factor (GM-CSF) can be administered as a single agent followed by concurrent administration with AMD3100. In particular embodiments, a treatment protocol includes a 5 day treatment where GM-CSF can be administered on day 1, day 2, day 3, and day 4 and on day 5, GM-CSF and AMD3100 are administered 6 to 8 hours prior to PB collection.

HSPC from PB can be collected from the blood through a syringe or catheter inserted into a subject's vein. For example, the PB can be collected using an apheresis machine. Blood flows from the vein through the catheter into an apheresis machine, which separates the white blood cells, including HSPC from the rest of the blood and then returns the remainder of the blood to the subject's body. Apheresis can be performed for several days (e.g., 1 to 5 days) until enough HSPC have been collected.

In particular embodiments, large volume leukapheresis is performed over two days, starting when circulating CD34+ cells are ≥10 cells/μL. On the first day, the obtained sample is diluted in an autologous plasma sample to a concentration of 5 200×10⁶ cells/mL. On the second day, the obtained sample is pooled with the first sample. The samples are washed to remove platelets, before or after pooling. In particular embodiments, large volume leukapheresis refers to processing 3 to 6 total blood volumes (TBV) of a donor or patient. In particular embodiments, large volume leukapheresis refers to processing 3 or greater TBV of a donor or patient. In particular embodiments, large volume leukapheresis refers to processing 4 or greater TBV of a donor or patient. In particular embodiments, large volume leukapheresis refers to processing 5 or greater TBV of a donor or patient. In particular embodiments, large volume leukapheresis differs from standard volume leukapheresis by processing a larger volume of blood, an increased blood flow rate, additional heparin anticoagulant and/or longer duration of the procedure. In particular embodiments, the systems and methods of the present disclosure can use standard volume leukapheresis.

Within the current disclosure, mobilization can be performed by treating the subject with any agent(s) described herein or known in the art, that increase the number of HSC circulating in the PB of the subject. Commonly used mobilization agents include G-CSF, GM-CSF, and AMD3100.

G-CSF is a cytokine whose functions in HSPC mobilization can include the promotion of granulocyte expansion and both protease-dependent and independent attenuation of adhesion molecules and disruption of the SDF-1/CXCR4 axis. In particular embodiments, any commercially available form of G-CSF can be used (e.g., Neupogen® and/or Neulasta®, Amgen Inc., Thousand Oaks, Calif.). In particular embodiments, G-CSF can include any of SEQ ID NOs: 1-4. In particular embodiments, an effective amount of G-CSF includes 0.1 μg/kg to 100 μg/kg. In particular embodiments, an effective amount of G-CSF includes 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, or 20 μg/kg. In particular embodiments, an effective amount of G-CSF includes 16 μg/kg.

GM-CSF is a monomeric glycoprotein also known as colony-stimulating factor 2 (CSF2) that functions as a cytokine and is naturally secreted by macrophages, T cells, mast cells, NK cells, endothelial cells, and fibroblasts. In particular embodiments, any commercially available form of GM-CSF can be used (e.g., Sargramostim (Leukine, Bayer Healthcare Pharmaceuticals, Seattle, Wash.) and molgramostim (Schering-Plough, Kenilworth, N.J.)). In particular embodiments, GM-CSF can include SEQ ID NO: 5. Effective amounts of GM-CSF to administer can include doses ranging from, for example, 0.1 to 50 μg/kg or from 0.5 to 30 μg/kg.

AMD3100 (also known as plerixafor; UMK121, AMD3000, GZ 316455, GZ316455, JM3100, and SDZSID791) is a synthetic organic molecule of the bicyclam class. It is a chemokine receptor antagonist that reversibly inhibits SDF-1 binding to CXCR4, promoting HSPC mobilization. AMD3100 is approved to be used in combination with G-CSF for HSPC mobilization in patients with myeloma and lymphoma. The structure of AMD3100 is:

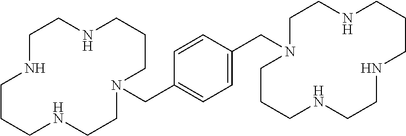 [SMILES: c1cc(CN2CCCNCCNCCCNCC2)ccc1CN1CCCNCCNCCCNCC1]

In particular embodiments, an effective amount of AMD3100 includes 0.1 mg/kg to 100 mg/kg. In particular embodiments, an effective amount of AMD3100 includes 100 μg/kg, 150 μg/kg, 200 μg/kg, 250 μg/kg, 300 μg/kg, or 350 μg/kg. In particular embodiments, an effective amount of AMD3100 is 240 μg/kg.

Stem Cell Factor (SCF; also known as KIT ligand, KL, c-kit ligand, mast cell growth factor, or steel factor) is a cytokine that binds to the c-kit receptor (CD117). SCF can exist both as a transmembrane protein and a soluble protein. This cytokine plays an important role in hematopoiesis, spermatogenesis, and melanogenesis. In particular embodiments, any commercially available form of SCF can be used (e.g., Ancestim, Stemgen®, Amgen Inc., Thousand Oaks, Calif.). In particular embodiments, SCF can include SEQ ID NO: 6. Effective amounts of SCF to administer can include doses ranging from, for example, 0.1 to 100 μg/kg or from 0.5 to 50 μg/kg.

In particular embodiments, PB can be mobilized by treating the subject with one or more cytokines or growth factors selected from interleukin (IL)-1, IL-7, IL-8, IL-11, Flt3 ligand, or thrombopoietin (TPO). In particular embodiments, PB is mobilized by treating the subject with one or more chemokines (e.g., macrophage inflammatory protein-1α (MIP1α)), chemokine receptor ligands (e.g., GROβ and/or GROβ_{Δ4} (King et al. (2001) Blood 97: 1534-1542)), and/or chemokine receptor analogs (e.g., stromal cell derived factor-1α (SDF-1α)).

In particular embodiments, PB is mobilized by treating the subject with one or more anti-integrin signaling agents. Anti-integrin signaling agents include anti-very late antigen 4 (VLA-4) inhibitors or antibodies (e.g., Natalizumab (Zohren et al. (2008) Blood 111: 3893-3895)); BIO5192 (Ramirez et al. (2009) Blood 114: 1340-1343), anti-vascular cell adhesion molecule 1 (VCAM-1); the α4β1 and α4β7 integrin inhibitor, α4β7 (Kim et al. (2016) Blood 128: 2457-2461); Vedolizumab (Rosario et al. (2016) Clin Drug Investig 36: 913-923); and N-(benzenesulfonyl)-L-prolyl-L-O-(1-pyrrolidinylcarbonyl) tyrosine (Cao et al. (2016) Nat Commun 7: 11007).

In particular embodiments, chemotherapeutic agents such as cyclophosphamide, etoposide, ifosfamide, cisplatin, cytarabine, and/or paclitaxel can be used as mobilization factors.

In particular embodiments, one or more mobilization factors can be administered subcutaneously or intravenously. In particular embodiments, one or more mobilization factors can be administered for 1 day, 2 consecutive days, 3 consecutive days, 4 consecutive days, 5 consecutive days, 6 consecutive days, or more. In particular embodiments, one or more mobilization factors can be administered until a designated number of CD34+ HSC are circulating in the PB.

For additional information and options regarding PB mobilization, see Richter et al. (2017) Transfus Med Hemother 44:151-164; Bendall & Bradstock (2014) Cytokine & Growth Factor Reviews 25: 355-367; Craddock et al., 1997, Blood 90(12):4779-4788; Jin et al., 2008, Journal of Translational Medicine 6:39; Pelus, 2008, Curr. Opin. Hematol. 15(4):285-292; Papayannopoulou et al., 1998, Blood 91(7):2231-2239; Tricot et al., 2008, Haematologica 93(11):1739-1742; Weaver et al., 2001, Bone Marrow Transplantation 27(2):S23-S29); WO2003/043651; WO2005/017160; WO2011/069336; U.S. Pat. Nos. 5,637,323; 7,288,521; 9,782,429; US2002/0142462; and US2010/02268.

(iii) Negative Selection to Yield CD34+ HSC Population with Accessory Cells. In particular embodiments, it is important to remove contaminating cell populations that might interfere with negative selection. For example, when the biological sample is BM, red blood cells (RBC) can be removed before negative selection. RBC can be removed through lysis using detergents, hetastarch, hetastarch with centrifugation, cell washing, cell washing with density gradient, Ficoll-hypaque, Sepx, Optipress, Filters, and other protocols that have been used both in the manufacture of HSC cell and/or gene therapies for research and therapeutic purposes. Particular embodiments utilize hetastarch sedimentation to deplete RBC. See, e.g., Rubinstein, et al. (1995). Proc-Natl-Acad-Sci-USA 92(22): 10119-22. In particular embodiments, hetastarch sedimentation can include adding 6% HES in 0.9% NaCl supplemented with 1 mM NAC to a blood or bone marrow sample at hematocrit value of 25%, allowing sedimentation for 30 minutes, and removing the RBC layer. In particular embodiments, the sedimentation is performed with a CliniMACS Prodigy™ device (Miltenyi Biotec GmbH, Auburn, Calif.).

When mobilized PB is used as a biological sample, platelets can be removed before negative selection through, for example, a platelet wash. In particular embodiments, a platelet wash can include performing a slow spin (120 g to 400 g) at room temperature on the buffy coats (white blood cells and platelets) obtained from a Ficoll-Paque density gradient centrifugation of a blood sample, removing and discarding the platelet-rich supernatant, resuspending the cell pellet in fresh buffer such as culture media or buffer solution (e.g., PBS), and repeating the steps at least twice for a total of 3 or more washes. In particular embodiments, the platelet washes are performed with a CliniMACS Prodigy™ device (Miltenyi Biotec GmbH, Auburn, Calif.). In particular embodiments, platelet washes can be done on leukapheresis products without a first buffy coat step. In particular embodiments, a platelet wash can include performing a slow spin (120 g to 400 g) at room temperature on a leukapheresis product, removing and discarding the platelet-rich supernatant, resuspending the cell pellet in fresh buffer such as culture media or buffer solution (e.g., PBS), and repeating the steps at least twice for a total of 3 or more washes.

Reference to CD34, CD45RA, CD3, CD14, CD16, and CD19 are understood by those of ordinary skill in the art. For other readers, CD (clusters of differentiation) antigens are proteins expressed on the surface of a cell that are detectable via specific binding proteins (e.g., antibodies or binding fragments thereof). In particular embodiments, sequences encoding CD antigens and sequences of CD antigens described herein can be SEQ ID NOs: 7-30. CD34 is a highly glycosylated type I transmembrane protein expressed on 1-4% of BM cells. CD45RA is related to fibronectin type III, has a molecular weight of 205-220 kDa and is expressed on B cells, naïve T cells, and monocytes. CD3 is a T cell co-receptor that helps to activate both cytotoxic T cells (CD8+ naïve T cells) and T helper cells (CD4+ naïve T cells). CD3 includes a protein complex including four distinct chains that are expressed at the surface of mature T cells. In mammals, the complex includes a CD3γ chain, a CD3δ chain, and two CD3ε chains. These chains associate with the T-cell receptor (TCR) and the chain (zeta-chain) to generate an activation signal in T cells. The TCR, ζ-chain, and CD3 molecules together constitute the TCR complex. CD14 is a component of the innate immune system and functions to recognize pathogen-associated molecular patterns, acting as a co-receptor with Toll-like receptor 4 (TLR4) and MD2 (lymphocyte antigen 96) to bind bacterial ligands such as lipopolysaccharide (LPS) and lipoteichoic acid. CD14 exists in two forms, a membrane-anchored form via a glycosylphosphatidylinositol tail (mCD14), and a soluble form (sCD14), which can appear after shedding of mCD14 (48 kDa) or can be directly secreted from intracellular vesicles (56 kDa). CD14 is expressed mainly by macrophages, with expression also found on neutrophils, dendritic cells, and enterocytes (intestinal absorptive cells). CD16 is a type III Fcγ membrane receptor (FcγRIII) found on the surface of NK cells, neutrophil polymorphonuclear leukocytes, monocytes, and macrophages. CD16 can bind the Fc portion of IgG antibodies and is involved in antibody-dependent cellular cytotoxicity (ADCC). CD16 exists as two forms, CD16a (FcγRIIIa) and CD16b (FcγRIIIb). CD19 is a transmembrane protein that in humans is expressed in all B lineage cells, except for plasma cells, and in follicular dendritic cells. In human B cells, CD19 can act as an adaptor protein to recruit cytoplasmic signaling proteins to the membrane and can also function in a CD19/CD21 complex to decrease the threshold for B cell receptor signaling pathways. Due to its presence on all B cells, CD19 is a biomarker for B cell development.

Antibodies to each of the selected CD markers are commercially available and known to those of ordinary skill in the art. For example, human antibodies against CD45RA include clone 5H9 (BD Horizon, San Jose, Calif.), clone JS-83 (Thermo Fisher Scientific, Waltham, Mass.), clone HI100 (Thermo Fisher Scientific, Waltham, Mass.), and anti-CD45RA [MEM-56] (Abcam, Cambridge, Mass.). Human antibodies against CD3 include clone UCHT1 (BD Biosciences, San Jose, Calif.), clone SK7 (Biolegend, San Diego, Calif.), clone OKT3 (Biolegend, San Diego, Calif.), and clone CD3-12 (Bio-Rad, Hercules, Calif.). Human antibodies against CD14 include clone 61D3 (Thermo Fisher Scientific, Pittsburgh, Pa.), clone HCD14 (Biolegend, San Diego, Calif.), clone 63D3 (Biolegend, San Diego, Calif.), and clone M5E2 (Biolegend, San Diego, Calif.). Human antibodies against CD16 include clone 3G8 (BD Biosciences, San Jose, Calif.), clone LNK16 (Bio-Rad, Hercules, Calif.), clone DJ130c (Bio-Rad, Hercules, Calif.), and clone KD1 (Bio-Rad, Hercules, Calif.). Human antibodies against CD 19 include clone 4G7 (BD Pharmingen, San Diego, Calif.), clone HIB19 (Biolegend, San Diego, Calif.), clone LT19 (Bio-Rad, Hercules, Calif.), and clone FMC63 (Millipore Sigma, Burlington, Mass.). Mouse antibodies for further experimental work are also available, as detailed in Example 1.

Depending on the marker combination selected for removal, the selected unwanted cells can be removed from a sample using any appropriate technique. Appropriate collection and isolation procedures include magnetic separation (e.g., using antibody-coated magnetic beads); fluorescence activated cell sorting (FACS); nanosorting based on fluorophore expression; affinity chromatography; cytotoxic agents joined to a monoclonal antibody or used in conjunction with a monoclonal antibody, e.g., complement and cytotoxins; “panning” with binding proteins (e.g., antibodies or binding fragments thereof) attached to a solid matrix; selective agglutination using a lectin such as soybean; or combinations of these techniques, etc.

In particular embodiments, a biological sample can be processed to remove selected unwanted cells using appropriate binding proteins against one or more of CD45RA, CD3, CD14, CD16, and/or CD19 directly or indirectly conjugated to magnetic particles in connection with a magnetic cell separator, for example, the CliniMACS® Cell Separation System (Miltenyi Biotec, Bergisch Gladbach, Germany). The CliniMACS® Cell Separation System employs nano-sized super-paramagnetic particles composed of iron oxide and dextran coupled to specific monoclonal antibodies. The CliniMACS® Cell Separator is a closed sterile system, outfitted with a single-use disposable tubing set. The disposable set can be used for and discarded after processing a single biological sample to remove selected unwanted cells.

In particular embodiments, cell populations can be removed based on light scattering properties of the cells based on side scatter channel (SSC) brightness and forward scatter channel (FSC) brightness. Side scatter refers to the amount of light scattered orthogonally (90° from the direction of the laser source), as measured by flow cytometry. Forward scatter refers to the amount of light scattered generally less than 90° from the direction of the light source. Generally, as cell granularity increases, the side scatter increases and as cell diameter increases, the forward scatter increases.

Side scatter and forward scatter are measured as intensity of light. Those skilled in the art recognize that the amount of side scatter can be differentiated with user-defined settings. In particular embodiments, low (10) side scatter refers to less than 50% intensity, less than 40% intensity, less than 30% intensity, or even less intensity, in the side scatter channel of the flow cytometer. Conversely high (hi) side scatter cells are the reciprocal population of cells that are not low side scatter. Forward scatter is defined in the same manner as side scatter but the light is collected in forward scatter channel. Thus, particular embodiments include removal of cell populations based on precise combinations of cell surface markers (CD markers) and the associated light scattering properties of the cells.

As is understood by one of ordinary skill in the art of flow cytometry, “hi”, “int”, “lo”, “+” and “−” refer to the intensity of a signal relative to negative or other populations. In particular embodiments, positive expression (+) means that the marker is detectable on a cell using flow cytometry. In particular embodiments, negative expression (−) means that the marker is not detectable using flow cytometry. In particular embodiments, “hi” means that the positive expression of a marker of interest is brighter as measured by fluorescence (using for example FACS) than other cells also positive for expression. In these embodiments, those of ordinary skill in the art recognize that brightness is based on a threshold of detection. Generally, one of skill in the art will analyze a negative control tube first, and set a gate (bitmap) around the population of interest by FSC and SSC and adjust the photomultiplier tube voltages and gains for fluorescence in the desired emission wavelengths, such that 97% of the cells appear unstained for the fluorescence marker with the negative control. Once these parameters are established, stained cells can be detected and/or removed. In particular embodiments, and representative of a typical FACS plot, hi implies to the farthest right (x line) or highest top line (upper right or left) while lo implies within the left lower quadrant or in the middle between the right and left quadrant (but shifted relative to the negative population). In particular embodiments, “hi” refers to greater than 20-fold of +, greater than 30-fold of +, greater than 40-fold of +, greater than 50-fold of +, greater than 60-fold of +, greater than 70-fold of +, greater than 80-fold of +, greater than 90-fold of +, greater than 100-fold of +, or more of an increase in detectable fluorescence relative to + cells. Conversely, “lo” can refer to a reciprocal population of those defined as “hi”.

Optionally, prior to selected unwanted cell removal, an aliquot of the biological sample can be checked for total nucleated cell count and/or CD34+ content. In a particular embodiment, after selected unwanted cell removal, CD34+ HSC are recovered. In particular embodiments, the CD34+ HSC are CD34+, CD45RA−, CD90+ HSC.

The CD34+ HSC sample derived from negative selection can be subsequently processed. In particular embodiments, CD34+ HSC processing includes culturing the CD34+ HSC in a RetroNectin™ (Takara Bio USA, Mountain View, Calif.)-coated culture flask at a density of 1×10⁶ cells/mL and 2.9×10⁵ cells/cm² in StemSpan™ (StemCell Technologies, Vancouver, British Columbia) media supplemented with 4 μg/mL protamine sulfate; 100 ng/mL human SCF; 100 ng/mL TPO; 100 ng/mL Flt-3 ligand; and 1 mM NAC.

In particular embodiments, CD34+ HSC processing includes culturing the CD34+ HSC in an appropriate cell culture medium for transport or storage. In particular embodiments, the cell culture medium consists of STEMSPAN™ Serum Free Expansion Medium supplemented with IL-3, IL-6, TPO, Flt-3L, and SCF.

In particular embodiments, HSC expansion can be performed. Expansion can occur in the presence of one or more growth factors, such as: angiopoietin-like proteins (Angptls, e.g., Angptl2, Angptl3, Angptl7, Angpt15, and Mfap4), erythropoietin, fibroblast growth factor-1 (FGF-1), FLT3-L, G-CSF, GM-CSF, insulin growth factor-2 (IFG-2), IL-3, IL-6, IL-7, IL-11, SCF, TPO, and analogs thereof (wherein the analogs include any structural variants of the growth factors having the biological activity of the naturally occurring growth factor; see, e.g., WO2007/1145227 and US2010/0183564). For clarity, growth factor agents can also be used as mobilizing agents. Particular embodiments utilize expansion in HSC supportive media (e.g. StemSpan) supplemented with either SCF, TPO, and FLT3-L or SCF and IL-3, or other combinations of growth factors.

In particular embodiments, the type, amount and/or concentration of growth factors suitable for expanding HSC is the amount or concentration effective to promote proliferation. HSC populations are preferably expanded until a sufficient number of cells are obtained to provide for at least one infusion into a human subject, typically around 10⁴ cells/kg to 10⁹ cells/kg.

The amount or concentration of growth factors suitable for expanding HSC depends on the activity of the growth factor preparation, and the species correspondence between the growth factors and HSC, etc. Generally, when the growth factor(s) and HSC are of the same species, the total amount of growth factor in the culture medium ranges from 1 ng/ml to 5 μg/ml, from 5 ng/ml to 1 μg/ml, or from 5 ng/ml to 250 ng/ml. In additional embodiments, the amount of growth factors can be in the range of 5-1000 or 50-100 ng/ml.

Additional methods to expand and/or maintain HSC in culture can utilize one or more of a commercially available base media such as StemSpan SFEM or ACF media (both from Stem Cell Technologies) or XVivo media types (Lonza) supplemented with one or more of: Cyto/chemokines (e.g., G-CSF, SCF, TPO, FLT3-L, IL-3, IL-6); small molecules such as aryl-hydrocarbon receptor antagonists (e.g., StemRegenin1 (Phenol, 4-[2-[[2-benzo[b]thien-3-yl-9-(1-methylethyl)-9H-purin-6-yl]amino]ethyl]); GNF351 (N-(2-(3H-Indol-3-yl)ethyl)-9-isopropyl-2-(5-methyl-3-pyridyl)-7H-purin-6-amine, N-(2-(1H-Indol-3-yl)ethyl)-9-isopropyl-2-(5-methylpyridin-3-yl)-9H-purin-6-amine); CH223191 (1-Methyl-N[2-methyl-4[2-(2-methylphenyl)diazenyl]phenyl-1H-pyrazole-5-carboxamide), pyrimidoindole derivatives (e.g., UM171 (1r,4r)-N1-(2-benzyl-7-(2-methyl-2H-tetrazol-5-yl)-9H-pyrimido[4,5-b]indol-4-yl)cyclohexane-1,4-diamine); UM729 (Methyl 4-((3-(piperidin-1-yl)propyl)amino)-9H-pyrimido[4,5-b] indole-7-carboxylate); UM118428 (Tranylcypromine HCl, (trans-2-Phenylcyclopropylamine hydrochloride)), and glucocorticoid receptor antagonists (e.g., mifepristone (RU-486), RU-43044, Miconazole, 11-oxa cortisol, 11-oxa prednisolone, Dexamethasone mesylate). Additional agents which could be utilized include protamine sulfate, rapamycin, polybrene, fibronectin fragment, prostaglandins or nonsteroidal anti-inflammatory drugs (e.g., celecoxib, diclofenac, diflunisal, etodolac, ibuprofen, indomethacin, ketoprofen, nabumetone, naproxen, oxaprozin, piroxicam, salsalate, sulindac, AND tolmetin). Endothelial cell co-culture may also be used.

(iv) Genetic Modification of Negatively-Selected CD34+ HSC Populations. Negatively-selected CD34+ HSC populations can be genetically modified, for example, for a research and/or therapeutic purpose. In particular embodiments, negatively-selected CD34+ HSC populations can be genetically modified to include a therapeutic gene and/or to produce a therapeutic gene product.

In particular embodiments, a gene refers to a polynucleotide that codes for a particular sequence of amino acids, which include all or part of one or more proteins, and may include regulatory (non-transcribed) DNA sequences, such as promoter sequences, which determine, for example, the conditions under which the gene is expressed. The transcribed region of the gene may include untranslated regions, including introns, 5′-untranslated region (UTR), and 3′-UTR, as well as the coding sequence. In particular embodiments, a gene can include enhancers, Kozak sequences, polyadenylation signals, restriction enzyme sites, multiple cloning sites, internal ribosomal entry sites (IRES), recombinase recognition sites (e.g., LoxP, FRT, and Att sites), termination codons, transcriptional termination signals, and epitope tags.

Particular examples of therapeutic genes and/or gene products to treat immune deficiencies can include genes associated with FA including: FancA, FancB, FancC, FancD1 (BRCA2), FancD2, FancE, FancF, FancG, FancI, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3). Exemplary genes and proteins associated with FA include: Homo sapiens FANCA coding sequence (SEQ ID NO: 31); Homo sapiens FANCC coding sequence (SEQ ID NO: 32); Homo sapiens FANCE coding sequence (SEQ ID NO: 33); Homo sapiens FANCF coding sequence (SEQ ID NO: 34); Homo sapiens FANCG coding sequence (SEQ ID NO: 35); Homo sapiens FANCA AA (SEQ ID NO: 36); Homo sapiens FANCC AA (SEQ ID NO: 37); Homo sapiens FANCE AA (SEQ ID NO: 38); Homo sapiens FANCF AA (SEQ ID NO: 39); and Homo sapiens FANCG AA (SEQ ID NO: 40).

Particular examples of therapeutic genes and/or gene products to treat immune deficiencies can include genes associated with SCID including: γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, and SLC46A1. Exemplary genes and proteins associated with SCID include: exemplary codon optimized Human γC DNA (SEQ ID NO: 41); exemplary native Human γC DNA (SEQ ID NO: 42); exemplary native canine γC DNA (SEQ ID NO: 43); exemplary human γC AA (SEQ ID NO: 44); and exemplary native canine γC AA (91% conserved with human) (SEQ ID NO: 45). Exemplary genes and proteins associated with SCID include: Homo sapiens JAK3 coding sequence (SEQ ID NO: 46); Homo sapiens PNP coding sequence (SEQ ID NO: 47); Homo sapiens ADA coding sequence (SEQ ID NO: 48); Homo sapiens RAG1 coding sequence (SEQ ID NO: 49); Homo sapiens RAG2 coding sequence (SEQ ID NO: 50); Homo sapiens JAK3 AA (SEQ ID NO: 51); Homo sapiens PNP AA (SEQ ID NO: 52); Homo sapiens ADA AA (SEQ ID NO: 53); Homo sapiens RAG1 AA (SEQ ID NO: 54); and Homo sapiens RAG2 AA (SEQ ID NO: 55).

Additional examples of therapeutic genes and/or gene products include those that can provide a therapeutically effective response against diseases related to red blood cells and clotting. In particular embodiments, the disease is a hemoglobinopathy like thalassemia, or a SCD/trait. Exemplary therapeutic genes include F8 and F9.

Additional examples of therapeutic genes and/or gene products include those that can provide a therapeutically effective response against a lysosomal storage disorder. In particular embodiments, the lysosomal storage disorder is mucopolysaccharidosis (MPS), type I; MPS II or Hunter Syndrome; MPS III or Sanfilippo syndrome; MPS IV or Morquio syndrome; MPS V; MPS VI or Maroteaux-Lamy syndrome; MPS VII or sly syndrome; alpha-mannosidosis; beta-mannosidosis; glycogen storage disease type I, also known as GSDI, von Gierke disease, or Tay Sachs; Pompe disease; Gaucher disease; or Fabry disease. The therapeutic gene may be, for example a gene encoding or inducing production of an enzyme, or that otherwise causes the degradation of mucopolysaccharides in lysosomes. Exemplary therapeutic genes include IDUA or iduronidase, IDS, GNS, HGSNAT, SGSH, NAGLU, GUSB, GALNS, GLB1, ARSB, and HYAL1.

Additional examples of therapeutic genes and/or gene products include those that can provide a therapeutically effective response against cancer. The therapeutic gene may be, for example, a tumor suppressor gene, a gene that induces apoptosis, a gene encoding an enzyme, a gene encoding an antibody, or a gene encoding a hormone. Exemplary therapeutic genes and gene products include 101F6, 123F2 (RASSF1), 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fms, FOX, FUS 1, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1 (HYAL1), LUCA-2 (HYAL2), LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, VVT1, WT-1, YES, and zac1.

Additional examples of therapeutic genes and/or gene products include those that can provide a therapeutically effective response against an infectious disease. In particular embodiments, the infectious disease is HIV. The therapeutic gene may be, for example, a gene rendering immune cells resistant to HIV infection, or which enables immune cells to effectively neutralize the virus via immune reconstruction, polymorphisms of genes encoding proteins expressed by immune cells, genes advantageous for fighting infection that are not expressed in the patient, genes encoding an infectious agent, receptor or coreceptor; a gene encoding ligands for receptors or coreceptors; viral and cellular genes essential for viral replication including; a gene encoding ribozymes, antisense RNA, small interfering RNA (siRNA) or decoy RNA to block the actions of certain transcription factors; a gene encoding dominant negative viral proteins, intracellular antibodies, intrakines and suicide genes. Exemplary therapeutic genes and gene products include α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC; and laminin receptor.

Additional examples of therapeutic genes and/or gene products include soluble CD40; CTLA; Fas L; antibodies to CD4, CD5, CD7, CD52, etc.; antibodies to IL1, 1L2, 1L6; an antibody to TCR specifically present on autoreactive T cells; IL4; MO; 1L12; 1L13; IL1Ra, sIL1RI, sIL1RII; sTNFRI; sTNFRII; antibodies to TNF; P53, PTPN22, and DRB1*1501/DQB1*0602; globin family genes; WAS; phox; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; and/or C9ORF72.

Therapeutic genes can be delivered using any appropriate method. In particular embodiments, therapeutic genes are delivered utilizing viral vectors.

In particular embodiments, viral-mediated gene transfer can utilize lentiviral vectors, adenoviral vectors, adeno-associated viral vectors, alpharetroviral vectors or gammaretroviral vectors.

Lentiviral vectors. “Lentivirus” refers to a genus of retroviruses that are capable of infecting dividing and non-dividing cells and typically produce high viral titers. Lentiviral vectors have been employed in gene therapy for a number of diseases. For example, hematopoietic gene therapies using lentiviral vectors or gamma retroviral vectors have been used for x-linked adrenoleukodystrophy and beta thalassemia. Several examples of lentiviruses include HIV (including HIV type 1, and HIV type 2); equine infectious anemia virus; feline immunodeficiency virus (FIV); bovine immune deficiency virus (BIV); and simian immunodeficiency virus (Sly).

Additional examples of viral vectors include those derived from adenoviruses (e.g., adenovirus 5 (Ad5), adenovirus 35 (Ad35), adenovirus 11 (Ad11), adenovirus 26 (Ad26), adenovirus 48 (Ad48) or adenovirus 50 (Ad50)), adeno-associated virus (AAV; see, e.g., U.S. Pat. No. 5,604,090), alphaviruses, cytomegaloviruses (CMV), flaviviruses, herpes viruses (e.g., herpes simplex), influenza viruses, papilloma viruses (e.g., human and bovine papilloma virus; see, e.g., U.S. Pat. No. 5,719,054), poxviruses, vaccinia viruses, modified vaccinia Ankara (MVA), NYVAC, or strains derived therefrom. Other examples include avipox vectors, such as a fowlpox vectors (e.g., FP9) or canarypox vectors (e.g., ALVAC and strains derived therefrom).

In particular embodiments, the efficiency of integration, the size of the DNA sequence that can be integrated, and the number of copies of a DNA sequence that can be integrated into a genome can be improved by using transposons. Transposons or transposable elements include a short nucleic acid sequence with terminal repeat sequences upstream and downstream. Active transposons can encode enzymes that facilitate the excision and insertion of nucleic acid into a target DNA sequence.

A number of transposable elements have been described in the art that facilitate insertion of nucleic acids into the genome of vertebrates, including humans. Examples include sleeping beauty (e.g., derived from the genome of salmonid fish); piggybac (e.g., derived from lepidopteran cells and/or the Myotis lucifugus); mariner (e.g., derived from Drosophila); frog prince (e.g., derived from Rana pipiens); Tol2 (e.g., derived from medaka fish); TcBuster (e.g., derived from the red flour beetle Tribolium castaneum) and spinON.

Other methods of gene delivery include use of artificial chromosome vectors such as mammalian artificial chromosomes (Vos, 1998) and yeast artificial chromosomes (YAC). YAC are typically used when the inserted nucleic acids are too large for more conventional vectors (e.g., greater than 12 kb).

In particular embodiments, the nucleic acid is stably integrated into the genome of a cell. In particular embodiments, the nucleic acid is stably maintained in a cell as a separate, episomal segment.

Vectors and other methods to deliver nucleic acids can include regulatory sequences to control the expression of the nucleic acid molecules. These regulatory sequences can be eukaryotic or prokaryotic in nature. In particular embodiments, the regulatory sequence can be a tissue specific promoter such that the expression of the one or more therapeutic proteins will be substantially greater in the target tissue type compared to other types of tissue. In particular embodiments, the regulatory sequence can result in the constitutive expression of the one or more therapeutic proteins upon entry of the vector into the cell. Alternatively, the regulatory sequences can include inducible sequences. Inducible regulatory sequences are well known to those skilled in the art and are those sequences that require the presence of an additional inducing factor to result in expression of the one or more therapeutic proteins. Examples of suitable regulatory sequences include binding sites corresponding to tissue-specific transcription factors based on endogenous nuclear proteins, sequences that direct expression in a specific cell type, the lac operator, the tetracycline operator and the steroid hormone operator. Any inducible regulatory sequence known to those of skill in the art may be used.

For additional information regarding procedures for genetic modification, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1:13-26 (1994).

(v) Cell Formulation. CD34+ HSC derived from negative selection can be formulated for administration to subjects. Exemplary carriers include saline, buffered saline, physiological saline, water, Hanks' solution, Ringer's solution, Nonnosol-R (Abbott Labs), Plasma-Lyte A® (Baxter Laboratories, Inc., Morton Grove, Ill.), glycerol, ethanol, and combinations thereof. In particular embodiments, cell-based formulations are administered to subjects as soon as reasonably possible following their initial formulation.

In particular embodiments, carriers can be supplemented with human serum albumin (HSA) or other human serum components or fetal bovine serum or other species serum components. In particular embodiments, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, cell-based formulations can include a local anesthetic such as lidocaine to ease pain at a site of injection.

Therapeutically effective amounts of cells within cell-based formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁶ cells, greater than 10¹⁰ cells, or greater than 10¹¹ cells.

In cell-based formulations disclosed herein, cells are generally in a volume of a liter or less, 500 ml or less, 250 ml or less, or 100 ml or less. Hence the density of administered cells is typically greater than 10⁴ cells/ml, 10⁷ cells/ml or 10⁸ cells/ml.

The cell-based formulations disclosed herein can be prepared for administration by, for example, injection, infusion, perfusion, or lavage.

In particular embodiments, it can be necessary or beneficial to cryopreserve a cell and/or cell-based formulation. The terms “frozen/freezing” and “cryopreserved/cryopreserving” can be used interchangeably. Freezing includes freeze drying. As is understood by one of ordinary skill in the art, the freezing of cells can be destructive (see Mazur, P., 1977, Cryobiology 14:251-272) but there are numerous procedures available to prevent such damage. For example, damage can be avoided by (a) use of a cryoprotective agent, (b) control of the freezing rate, and/or (c) storage at a temperature sufficiently low to minimize degradative reactions. Exemplary cryoprotective agents include dimethyl sulfoxide (DMSO) (Lovelock and Bishop, 1959, Nature 183:1394-1395; Ashwood-Smith, 1961, Nature 190:1204-1205), glycerol, polyvinylpyrrolidine (Rinfret, 1960, Ann. N.Y. Acad. Sci. 85:576), polyethylene glycol (Sloviter and Ravdin, 1962, Nature 196:548), albumin, dextran, sucrose, ethylene glycol, i-erythritol, D-ribitol, D-mannitol (Rowe et al., 1962), D-sorbitol, i-inositol, D-lactose, choline chloride (Bender et al., 1960), amino acids (Phan The Tran and Bender, 1960), methanol, acetamide, glycerol monoacetate (Lovelock, 1954), and inorganic salts (Phan The Tran and Bender, 1960; Phan The Tran and Bender, 1961). In particular embodiments, DMSO can be used. Addition of plasma (e.g., to a concentration of 20-25%) can augment the protective effects of DMSO. After addition of DMSO, cells can be kept at 0° C. until freezing, because DMSO concentrations of 1% can be toxic at temperatures above 4° C.

In the cryopreservation of cells, slow controlled cooling rates can be critical and different cryoprotective agents and different cell types have different optimal cooling rates. The heat of fusion phase where water turns to ice should be minimal. The cooling procedure can be carried out by use of, e.g., a programmable freezing device or a methanol bath procedure. Programmable freezing apparatuses allow determination of optimal cooling rates and facilitate standard reproducible cooling.

In particular embodiments, DMSO-treated cells can be pre-cooled on ice and transferred to a tray containing chilled methanol which is placed, in turn, in a mechanical refrigerator (e.g., Harris or Revco) at −80° C. Thermocouple measurements of the methanol bath and the samples indicate a cooling rate of 1° to 3° C./minute can be preferred. After at least two hours, the specimens can have reached a temperature of −80° C. and can be placed directly into liquid nitrogen (−196° C.).

After thorough freezing, the cells can be rapidly transferred to a long-term cryogenic storage vessel. In a preferred embodiment, samples can be cryogenically stored in liquid nitrogen (−196° C.) or vapor (−1° C.). Such storage is facilitated by the availability of highly efficient liquid nitrogen refrigerators.

Further considerations and procedures for the manipulation, cryopreservation, and long term storage of cells, can be found in the following exemplary references: U.S. Pat. Nos. 4,199,022; 3,753,357; and 4,559,298; Gorin, 1986; Bone-Marrow Conservation, Culture and Transplantation, Proceedings of a Panel, Moscow, Jul. 22-26, 1968, International Atomic Energy Agency, Vienna, pp. 107-186).

Following cryopreservation, frozen cells can be thawed for use in accordance with methods known to those of ordinary skill in the art. Frozen cells are preferably thawed quickly and chilled immediately upon thawing. In particular embodiments, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed on ice.

In particular embodiments, methods can be used to prevent cellular clumping during thawing. Exemplary methods include: the addition before and/or after freezing of DNase, low molecular weight dextran and citrate, hydroxyethyl starch, etc.

As is understood by one of ordinary skill in the art, if a cryoprotective agent that is toxic to humans is used, it should be removed prior to therapeutic use. DMSO has no serious toxicity.

(vi) Methods of Use. The formulations disclosed herein can be used for treating subjects (humans, veterinary animals (dogs, cats, reptiles, birds, etc.), livestock (horses, cattle, goats, pigs, chickens, etc.), and research animals (monkeys, rats, mice, fish, etc.). In particular embodiments, subjects are human patients. Treating subjects includes delivering therapeutically effective amounts. Therapeutically effective amounts include those that provide effective amounts, prophylactic treatments, and/or therapeutic treatments.

An “effective amount” is the amount of a formulation necessary to result in a desired physiological change in a subject. Effective amounts are often administered for research purposes.

A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition.

A “therapeutic treatment” includes a treatment administered to a subject who displays symptoms or signs of a condition and is administered to the subject for the purpose of reducing the severity or progression of the condition.

The actual dose and amount of a therapeutic formulation administered to a particular subject can be determined by a physician, veterinarian, or researcher taking into account parameters such as physical and physiological factors including target; body weight; type of condition; severity of condition; upcoming relevant events, when known; previous or concurrent therapeutic interventions; idiopathy of the subject; and route of administration, for example. In addition, in vitro and in vivo assays can optionally be employed to help identify optimal dosage ranges.

Therapeutically effective amounts can be administered through any appropriate administration route such as by, injection, infusion, perfusion, or lavage.

In particular embodiments, methods of the present disclosure can be used to treat FA. In particular embodiments, therapeutic efficacy can be observed through lymphocyte reconstitution, improved clonal diversity and thymopoiesis, reduced infections, and/or improved patient outcome. Therapeutic efficacy can also be observed through one or more of weight gain and growth, improved gastrointestinal function (e.g., reduced diarrhea), reduced upper respiratory symptoms, reduced fungal infections of the mouth (thrush), reduced incidences and severity of pneumonia, reduced meningitis and blood stream infections, and reduced ear infections. In particular embodiments, treating FA with methods of the present disclosure include increasing resistance of BM derived cells to mitomycin C (MMC). In particular embodiments, the resistance of BM derived cells to MMC can be measured by a cell survival assay in methylcellulose and MMC.

In particular embodiments, methods of the present disclosure can be used to treat SCID. In particular embodiments, therapeutic efficacy can be observed through lymphocyte reconstitution, improved clonal diversity and thymopoiesis, reduced infections, and/or improved patient outcome. Therapeutic efficacy can also be observed through one or more of weight gain and growth, improved gastrointestinal function (e.g., reduced diarrhea), reduced upper respiratory symptoms, reduced fungal infections of the mouth (thrush), reduced incidences and severity of pneumonia, reduced meningitis and blood stream infections, and reduced ear infections.

In particular embodiments, methods of the present disclosure can be used to treat hypogammaglobulinemia. Hypogammaglobulinemia is caused by a lack of B-lymphocytes and is characterized by low levels of antibodies in the blood. Hypogammaglobulinemia can occur in patients with chronic lymphocytic leukemia (CLL), multiple myeloma (MM), non-Hodgkin's lymphoma (NHL) and other relevant malignancies as a result of both leukemia-related immune dysfunction and therapy-related immunosuppression. Patients with acquired hypogammaglobulinemia secondary to such hematological malignancies, and those patients receiving post-HSPC transplantation are susceptible to bacterial infections. The deficiency in humoral immunity is largely responsible for the increased risk of infection-related morbidity and mortality in these patients, especially by encapsulated microorganisms. For example, Streptococcus pneumoniae, Haemophilus influenzae, and Staphylococcus aureus, as well as Legionella and Nocardia spp. are frequent bacterial pathogens that cause pneumonia in patients with CLL. Opportunistic infections such as Pneumocystis carinii, fungi, viruses, and mycobacteria also have been observed. The number and severity of infections in these patients can be significantly reduced by administration of immune globulin (Griffiths H et al. (1989) Blood 73: 366-368; Chapel H M et al. (1994) Lancet 343: 1059-1063).

In particular embodiments, methods of the present disclosure can restore BM function in a subject in need thereof. In particular embodiments, restoring BM function can include improving BM repopulation with gene corrected cells as compared to a subject in need thereof not administered a therapy described herein. Improving BM repopulation with gene corrected cells can include increasing the percentage of cells that are gene corrected. In particular embodiments, the cells are selected from white blood cells and BM derived cells. In particular embodiments, the percentage of cells that are gene corrected can be measured using an assay selected from quantitative real time PCR and flow cytometry.

In particular embodiments, methods of the present disclosure can normalize primary and secondary antibody responses to immunization in a subject in need thereof. Normalizing primary and secondary antibody responses to immunization can include restoring B-cell and/or T-cell cytokine signaling programs functioning in class switching and memory response to an antigen. Normalizing primary and secondary antibody responses to immunization can be measured by a bacteriophage immunization assay. In particular embodiments, restoration of B-cell and/or T-cell cytokine signaling programs can be assayed after immunization with the T-cell dependent neoantigen bacteriophage φX174. In particular embodiments, normalizing primary and secondary antibody responses to immunization can include increasing the level of IgA, IgM, and/or IgG in a subject in need thereof to a level comparable to a reference level derived from a control population. In particular embodiments, normalizing primary and secondary antibody responses to immunization can include increasing the level of IgA, IgM, and/or IgG in a subject in need thereof to a level greater than that of a subject in need thereof not administered a gene therapy described herein. The level of IgA, IgM, and/or IgG can be measured by, for example, an immunoglobulin test. In particular embodiments, the immunoglobulin test includes antibodies binding IgG, IgA, IgM, kappa light chain, lambda light chain, and/or heavy chain. In particular embodiments, the immunoglobulin test includes serum protein electrophoresis, immunoelectrophoresis, radial immunodiffusion, nephelometry and turbidimetry. Commercially available immunoglobulin test kits include MININEPH™ (Binding site, Birmingham, UK), and immunoglobulin test systems from Dako (Denmark) and Dade Behring (Marburg, Germany). In particular embodiments, a sample that can be used to measure immunoglobulin levels includes a blood sample, a plasma sample, a cerebrospinal fluid sample, and a urine sample.

In particular embodiments, formulations are administered to subjects to treat acute lymphoblastic leukemia (ALL), acute myelogenous leukemia (AML), adrenoleukodystrophy, agnogenic myeloid metaplasia, amegakaryocytosis/congenital thrombocytopenia, ataxia telangiectasia, β-thalassemia major, chronic granulomatous disease, CLL, chronic myelogenous leukemia (CML), chronic myelomonocytic leukemia, common variable immune deficiency (CVID), complement disorders, congenital agammaglobulinemia, Diamond Blackfan syndrome, familial erythrophagocytic lymphohistiocytosis, Hodgkin's lymphoma, Hurler's syndrome, hyper IgM, IgG subclass deficiency, juvenile myelomonocytic leukemia, metachromatic leukodystrophy, mucopolysaccharidoses, multiple myeloma, myelodysplasia, non-Hodgkin's lymphoma, paroxysmal nocturnal hemoglobinuria (PNH), primary immunodeficiency diseases with antibody deficiency, pure red cell aplasia, refractory anemia, Shwachmann-Diamond-Blackfan anemia, selective IgA deficiency, severe aplastic anemia, SCD, specific antibody deficiency, Wiskott-Aldridge syndrome, and/or X-linked agammaglobulinemia (XLA).

Particular embodiments include treatment of secondary, or acquired, immune deficiencies such as immune deficiencies caused by trauma, viruses, chemotherapy, toxins, and pollution. As previously indicated, AIDS is an example of a secondary immune deficiency disorder caused by a virus, the HIV, in which a depletion of T lymphocytes renders the body unable to fight infection. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject, or decrease or eliminate HIV infectivity and/or virulence.

In particular embodiments, methods of the present disclosure can improve the kinetics and/or clonal diversity of lymphocyte reconstitution in a subject in need thereof. In particular embodiments, improving the kinetics of lymphocyte reconstitution can include increasing the number of circulating T lymphocytes to within a range of a reference level derived from a control population. In particular embodiments, improving the kinetics of lymphocyte reconstitution can include increasing the absolute CD3+ lymphocyte count to within a range of a reference level derived from a control population. A range of a reference level can be a range of values observed in or exhibited by normal (i.e., non-immuno-compromised) subjects for a given parameter. In particular embodiments, improving the kinetics of lymphocyte reconstitution can include reducing the time required to reach normal lymphocyte counts as compared to a subject in need thereof not administered a therapy described herein. In particular embodiments, improving the kinetics of lymphocyte reconstitution can include increasing the frequency of gene corrected lymphocytes as compared to a subject in need thereof not administered a therapy described herein. In particular embodiments, improving the kinetics of lymphocyte reconstitution can include increasing diversity of clonal repertoire of gene corrected lymphocytes in the subject as compared to a subject in need thereof not administered a gene therapy described herein. Increasing diversity of clonal repertoire of gene corrected lymphocytes can include increasing the number of unique retroviral integration site (RIS) clones as measured by a RIS analysis.

In particular embodiments, methods of the present disclosure can restore T-cell mediated immune responses in a subject in need thereof. Restoration of T-cell mediated immune responses can include restoring thymic output and/or restoring normal T lymphocyte development.

In particular embodiments, restoring thymic output can include restoring the frequency of CD3+ T cells expressing CD45RA in PB to a level comparable to that of a reference level derived from a control population. In particular embodiments, restoring thymic output can include restoring the number of T cell receptor excision circles (TRECs) per 10⁶ maturing T cells to a level comparable to that of a reference level derived from a control population. The number of TRECs per 10⁶ maturing T cells can be determined as described in Kennedy D R et al. (2011) Vet Immunol Immunopathol 142: 36-48.

In particular embodiments, restoring normal T lymphocyte development includes restoring the ratio of CD4+ cells:CD8+ cells to 2. In particular embodiments, restoring normal T lymphocyte development includes detecting the presence of αβ TCR in circulating T-lymphocytes. The presence of αβ TCR in circulating T-lymphocytes can be detected, for example, by flow cytometry using antibodies that bind an α and/or β chain of a TCR. In particular embodiments, restoring normal T lymphocyte development includes detecting the presence of a diverse TCR repertoire comparable to that of a reference level derived from a control population. TCR diversity can be assessed by TCRVβ spectratyping, which analyzes genetic rearrangement of the variable region of the TCRβ gene. Robust, normal spectratype profiles can be characterized by a Gaussian distribution of fragments sized across 17 families of TCRVβ segments. In particular embodiments, restoring normal T lymphocyte development includes restoring T-cell specific signaling pathways. Restoration of T-cell specific signaling pathways can be assessed by lymphocyte proliferation following exposure to the T cell mitogen phytohemagglutinin (PHA). In particular embodiments, restoring normal T lymphocyte development includes restoring white blood cell count, neutrophil cell count, monocyte cell count, lymphocyte cell count, and/or platelet cell count to a level comparable to a reference level derived from a control population.

In particular embodiments, therapeutically effective amounts may provide function to immune and other blood cells, reduce or eliminate an immune-mediated condition; and/or reduce or eliminate a symptom of the immune-mediated condition.

In particular embodiments, particular methods of use include in the treatment of conditions where corrected cells have a selective advantage over non-corrected cells. For example, in FA and SCID, corrected cells have an advantage and only transducing the therapeutic gene into a “few” HSPCs is sufficient for therapeutic efficacy.

(vii) Reference Levels Derived from Control Populations. Obtained values for parameters associated with a therapy described herein can be compared to a reference level derived from a control population, and this comparison can indicate whether a therapy described herein is effective for a subject in need thereof. Reference levels can be obtained from one or more relevant datasets from a control population. A “dataset” as used herein is a set of numerical values resulting from evaluation of a sample (or population of samples) under a desired condition. The values of the dataset can be obtained, for example, by experimentally obtaining measures from a sample and constructing a dataset from these measurements. As is understood by one of ordinary skill in the art, the reference level can be based on e.g., any mathematical or statistical formula useful and known in the art for arriving at a meaningful aggregate reference level from a collection of individual data points; e.g., mean, median, median of the mean, etc. Alternatively, a reference level or dataset to create a reference level can be obtained from a service provider such as a laboratory, or from a database or a server on which the dataset has been stored.

A reference level from a dataset can be derived from previous measures derived from a control population. A “control population” is any grouping of subjects or samples of like specified characteristics. The grouping could be according to, for example, clinical parameters, clinical assessments, therapeutic regimens, disease status, severity of condition, etc. In particular embodiments, the grouping is based on age range (e.g., 0-2 years) and non-immunocompromised status. In particular embodiments, a normal control population includes individuals that are age-matched to a test subject and non-immune compromised. In particular embodiments, age-matched includes, e.g., 0-6 months old; 0-1 year old; 0-2 years old; 0-3 years old; 10-15 years old, as is clinically relevant under the circumstances. In particular embodiments, a control population can include those that have an immune deficiency and have not been administered a therapeutically effective amount

In particular embodiments, the relevant reference level for values of a particular parameter associated with a therapy described herein is obtained based on the value of a particular corresponding parameter associated with a therapy in a control population to determine whether a therapy disclosed herein has been therapeutically effective for a subject in need thereof.

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular data point, where the data point is the result of random chance alone. A result is often considered significant (not random chance) at a p-value less than or equal to 0.05. In particular embodiments, a sample value is “comparable to” a reference level derived from a normal control population if the sample value and the reference level are not statistically significantly different.

(viii) Kits. Combinations of components to practice the methods disclosed herein can be used to create kits to create and/or use CD34+ HSC derived from negative selection.

In particular embodiments, the kits include medical supplies to obtain a BM sample or a mobilized PB sample. In particular embodiments, the kits include G-CSF/Filgrastim (Amgen Inc., Thousand Oaks, Calif.), GM-CSF, AMD3100 (Sigma-Aldrich, St. Louis, Mo.), SCF, and/or a chemotherapeutic agent. In particular embodiments, the kits include G-CSF/Filgrastim (Amgen Inc., Thousand Oaks, Calif.) and AMD3100 (Sigma-Aldrich, St. Louis, Mo.).

In particular embodiments, the kits include one or more proteins (e.g., antibodies) that bind CD45RA, CD3, CD14, CD16, and/or CD19. In particular embodiments, the kits include one or more of anti-CD45RA antibodies (e.g., clone 5H9), anti-CD3 antibodies (e.g., clone UCHT1), anti-CD14 antibodies (e.g., clone 61D3 anti-CD16 antibodies (e.g., clone 3G8), anti-CD19 antibodies (e.g., clone 4G7). In particular embodiments, the kits include magnetic elements and/or fluorescent elements.

In particular embodiments, the kits include a tubing set.

In particular embodiments, the kits include StemSpan™. In particular embodiments, the kits include protamine sulfate. In particular embodiments, the kits include human SCF. In particular embodiments, the kits include TPO. In particular embodiments, the kits include Flt-3 ligand. In particular embodiments, the kits include NAC.

In particular embodiments, the kits include components for a genetic therapy, such as a viral vector. In particular embodiments, the kits include a lentiviral vector. In particular embodiments, the kits include a nucleic acid encoding a therapeutic gene disclosed herein (e.g., a therapeutic FA gene).

In particular embodiments, the kits include a media to formulate cells for administration to a subject.

Kits can also include a notice in the form prescribed by a governmental agency regulating the manufacture, use, or sale of pharmaceuticals or biological products, which notice reflects approval by the agency of manufacture, use, or sale for human administration. The notice may state that the provided active ingredients can be administered to a subject. The kits can include further instructions for using the kit, for example, instructions regarding administration of the formulation and/or mobilization factors. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website. In particular embodiments, kits can also include some or all of the necessary medical supplies needed to use the kit effectively, such as syringes, ampules, tubing, facemask, an injection cap, sponges, sterile adhesive strips, Chloraprep, gloves, and the like. Variations in contents of any of the kits described herein can be made.

### Exemplary Embodiments

1. A method including: forming a CD34+ hematopoietic stem cell (HSC) population derived from negative selection from a biological sample.
2. A method of embodiment 1 wherein the negative selection removes only CD45RA+ cells.
3. A method of embodiment 1 wherein the negative selection removes one or more of CD45RA+ cells, CD3+ cells, CD14+ cells, CD16+ cells, and CD19+ cells.
4. A method of embodiment 1 wherein the negative selection removes CD45RA+ cells and CD3+ cells.
5. A method of embodiment 1 wherein the negative selection removes CD45RA+ cells and CD14+ cells.
6. A method of embodiment 1 wherein the negative selection removes CD45RA+ cells and CD16+ cells.
7. A method of embodiment 1 wherein the negative selection removes CD45RA+ cells and CD19+ cells.
8. A method of embodiment 1 wherein the negative selection removes CD45RA+, CD3+ cells, CD14+ cells, CD16+ cells and CD19+ cells.
9. A method of embodiment 1 wherein the negative selection removes only CD3+ cells, CD14+ cells, CD16+ cells and CD19+ cells.
10. A method of any of embodiments 1-9, wherein the negative selection includes contacting the biological sample with binding proteins that bind the CD marker associated with the removed cell type.
11. A method of embodiment 10, wherein the binding proteins include antibodies or binding fragments thereof.
12. A method of embodiment 11, wherein the antibodies include clone 5H9, clone JS-83, clone HI100, anti-CD45RA [MEM-56], clone UCHT1, clone SK7, clone OKT3, clone CD3-12, clone 61 D3, clone HCD14, clone 63D3, clone M5E2, clone 3G8, clone LNK16, clone DJ130c, clone KD1, clone 4G7, clone HIB19, clone LT19, and/or clone FMC63.
13. A method of embodiment 12, wherein the antibodies include clone 5H9, clone UCHT1, clone 61 D3, clone 3G8, and clone 4G7.
14. A method of any of embodiments 1-13, wherein the negative selection includes performing magnetic separation, fluorescence activated cell sorting (FACs), nanosorting, affinity chromatography, panning, and/or selective agglutination.
15. A method of any of embodiments 10-14, wherein the binding proteins are coupled to magnetic beads, fluorophores, and/or affinity tags.
16. A method of any of embodiments 1-15, wherein the negative selection results in at least a 70%, at least an 80%, or at least a 90% reduction in CD3+ cells, CD14+ cells, CD16+ cells, and CD19+ cells, collectively.
17. A method of any of embodiments 1-15, wherein the negative selection results in at least a 70%, at least an 80%, or at least a 90% reduction in lineage+ cells selected from CD3+ cells, CD14+ cells, CD16+ cells, and/or CD19+ cells.
18. A method of any of embodiments 1-17, wherein the negative selection retains at least 50%, at least 70%, or at least 90% of starting CD34+ cells in the biological sample.
19. A method of embodiment 18, wherein the retained CD34+ cells are CD34+, CD45RA−, and CD90+.
20. A method of any of embodiments 1-19, wherein cells retained in the biological sample following the negative selection include mesenchymal stem cells.
21. A method of any of embodiments 1-20, further including culturing CD34+ cells retained in the biological sample after negative selection in culture media.
22. A method of embodiment 21, wherein the culture media is supplemented with protamine sulfate, stem cell factor, thrombopoietin, Flt-3 ligand, and N-acetylcysteine (NAC).
23. A method of any of embodiments 18-22, wherein the CD34+ cells are cultured in fibronectin fragment-coated culture flasks.
24. A method of any of embodiments 1-23, wherein the biological sample is a bone marrow sample or a mobilized peripheral blood sample.
25. A method of any of embodiments 1-24, further including depleting the biological sample of red blood cells (RBCs) before the negative selection.
26. A method of any of embodiments 1-25, wherein the biological sample is a bone marrow sample diluted in cell culture media including NAC to obtain a hematocrit value of ≤25%.
27. A method of any of embodiments 1-26, further including genetically modifying CD34+ cells within the CD34+ cell population derived from negative selection.
28. A method of embodiment 27, wherein the genetic modification inserts a nucleic acid encoding a therapeutic gene.
29. A method of embodiment 28, wherein the therapeutic gene is selected from one or more of FancA, FancB, FancC, FancD1, FancD2, FancE, FancF, FancG, FancI, FancJ, FancL, FancM, FancN, FancO, FancP, FancQ, FancR, FancS, FancT, FancU, FancV, FancW, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, SLC46A1, F8, F9, IDUA, iduronidase, IDS, GNS, HGSNAT, SGSH, NAGLU, GUSB, GALNS, GLB1, ARSB, HYAL1, 101F6, 123F2, 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fms, FOX, FUS 1, FUS1, FYN, G-CSF, GDAIF, Gene 21, Gene 26, GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1, LUCA-2, LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, VVT1, WT-1, YES, zac1, α2β1, αvβ3, αvβ5, αvβ63, BOB/GPR15, Bonzo/STRL-33/TYMSTR, CCR2, CCR3, CCR5, CCR8, CD4, CD46, CD55, CXCR4, aminopeptidase-N, HHV-7, ICAM, ICAM-1, PRR2/HveB, HveA, α-dystroglycan, LDLR/α2MR/LRP, PVR, PRR1/HveC, laminin receptor, soluble CD40, CTLA, Fas L, globin family genes, WAS, phox, dystrophin, pyruvate kinase, CLN3, ABCD1, arylsulfatase A, SFTPB, SFTPC, NLX2.1, ABCA3, GATA1, ribosomal protein genes, TERT, TERC, DKC1, TINF2, CFTR, LRRK2, PARK2, PARK7, PINK1, SNCA, PSEN1, PSEN2, APP, SOD1, TDP43, FUS, ubiquilin 2, C9ORF72, and/or antibodies to CD4, CD5, CD7, CD52, IL-1, IL-2, IL-6; TNF; P53, PTPN22, DRB1*1501/DQB1*0602or a TCR specifically present on autoreactive T cells; IL-4; IL-10; IL-12; IL-13; IL-1Ra, sIL-1RI, sIL-1RII; sTNFRI; and/or sTNFRII.
30. A method of embodiment 28 or 29, wherein the nucleic acid includes a sequence selected from SEQ ID NOs: 31-35, 41-43, and/or 46-50.
31. A method of any of embodiments 28-30, wherein the nucleic acid encodes a protein selected from SEQ ID NOs: 36-40, 44, 45, and/or 51-55.
32. A method of any of embodiments 28-31, wherein a nucleic acid encoding the therapeutic gene includes a transposable element.
33. A method of any of embodiments 27-32, wherein the genetic modification utilizes a viral vector.
34. A method of embodiment 33, wherein the viral vector is selected from a lentiviral vector.
35. A method of any of embodiments 27-34 wherein the genetic modification includes transducing a viral vector at a multiplicity of infection (MOI) of 5 to 10 infectious units (IU)/cell.
36. A method of embodiments 34 or 35, wherein the lentiviral vector is a self-inactivating lentiviral vector.
37. A method of any of embodiments 34-36, wherein the lentiviral vector includes a human phosphoglycerate kinase (PGK) promoter.
38. A method of embodiment 37, wherein the human PGK promoter includes SEQ ID NO: 56.
39. A method of any of embodiments 1-38, further including formulating the CD34+ cell population derived from negative selection for administration to a subject.
40. A method of embodiment 39, wherein the formulated CD34+ cells have colony forming potential comparable to a reference population of CD34+ cells.
41. A method of embodiment 40, wherein the colony forming potential is measured by a colony-forming cell (CFC) assay.
42. A method of any of embodiments 39-41, wherein administration of the formulated CD34+ cells leads to CD45+ cell engraftment, T cell engraftment, monocyte engraftment, and/or B cell engraftment in a subject comparable to a reference population of CD34+ cells.
43. A method of embodiment 42, wherein the reference population of CD34+ cells are cells that have undergone positive selection for CD34+ marker.
44. A therapeutic cell formulation made by the method of any of embodiments 1-43.
45. A method including treating a subject with a therapeutic cell formulation of embodiment 44 by administering the therapeutic formulation to the subject.
46. A method of embodiment 45 wherein the subject has a fragile CD34+ cell population.
47. A method of embodiment 45 or 46, wherein the subject has a CD34^{lo} cell population.
48. A method of any of embodiments 45-47, wherein the subject has Fanconi anemia (FA), severe combined immunodeficiency (SCID), sickle cell disease (SCD), or Dyskeratosis congenita.
49. A method of any of embodiment 45-47, wherein the subject has a primary immune deficiency, a secondary immune deficiency, and/or cancer.
50. A method of embodiment 49, wherein the secondary immune deficiency is caused by HIV.
51. A method of embodiment 49, wherein the cancer is a leukemia.
52. A method of any of embodiments 45-51, further including administering to the subject mobilization factors.
53. A method of embodiment 52, wherein mobilization factors are administered on consecutive days until a designated number of CD34+ cells are detected circulating in the peripheral blood.
54. A method of embodiment 53, wherein the designated number is ≥10 CD34+ cells/μL.
55. A method of any of embodiments 52-54, wherein the mobilization factors are selected from G-CSF, GM-CSF, SCF, AMD3100, and/or a chemotherapeutic agent.
56. A method of any of embodiments 52-55, wherein the administering mobilization factors includes administering G-CSF on day 1, day 2, day 3, day 4, day 5, and day 6, and administering AMD3100 on day 4, day 5, and day 6.
57. A method of any of embodiments 52-56, wherein the administering mobilization factors includes administering 16 μg/kg/day G-CSF on day 1, day 2, day 3, day 4, day 5, and day 6, and administering 240 μg/kg/day plerixafor on day 4, day 5, and day 6.
58. A method of any of embodiments 52-57, wherein the administering mobilization factors includes subcutaneous administration.
59. A method of any of embodiments 45-58, further including performing leukapheresis.
60. A method of any of embodiment 59, wherein the leukapheresis occurs over two days.
61. A method of embodiment 59 or 60, wherein the leukapheresis is large volume leukapheresis.
62. A method of any of embodiments 59-61, wherein the collected mobilized peripheral blood sample is diluted in autologous plasma.
63. A method of embodiment 59-62, wherein the collected mobilized peripheral blood sample is diluted in autologous plasma to a concentration of ≤200×10⁶ cells/mL on day 1.
64. A method of any of embodiments 59-63, wherein the collected mobilized peripheral blood sample on day 2 is pooled with the diluted day 1 sample.
65. A method of any of embodiments 59-64, further including performing a platelet wash after the leukapheresis.
66. A method of any of embodiments 45-51, further including obtaining a bone marrow sample from the subject.
67. A method of embodiment 66, further including performing hetastarch sedimentation on the bone marrow sample to remove red blood cells (RBC).

Variants of protein and/or nucleic acid sequences disclosed herein can also be used. Variants include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein and nucleic acid sequences described or disclosed herein wherein the variant exhibits substantially similar or improved biological function.

“% sequence identity” refers to a relationship between two or more sequences, as determined by comparing the sequences. In the art, “identity” also means the degree of sequence relatedness between protein and nucleic acid sequences as determined by the match between strings of such sequences. “Identity” (often referred to as “similarity”) can be readily calculated by known methods, including those described in: Computational Molecular Biology (Lesk, A. M., ed.) Oxford University Press, N.Y. (1988); Biocomputing: Informatics and Genome Projects (Smith, D. W., ed.) Academic Press, N.Y. (1994); Computer Analysis of Sequence Data, Part I (Griffin, A. M., and Griffin, H. G., eds.) Humana Press, N.J. (1994); Sequence Analysis in Molecular Biology (Von Heijne, G., ed.) Academic Press (1987); and Sequence Analysis Primer (Gribskov, M. and Devereux, J., eds.) Oxford University Press, N.Y. (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” will mean any set of values or parameters, which originally load with the software when first initialized.

### Example 1

Lineage Depletion Preserves Autologous Blood HSC for Gene Therapy. The genetic basis of Fanconi anemia (FA) is a mutation in any one of 19 genes whose protein components make up the FA/breast cancer pathway responsible for DNA repair of inter-strand crosslinks through nucleotide excision followed by homologous recombination. Resulting compromises in genetic integrity are associated with a nearly uniform decline in hematopoietic stem and progenitor cells (HSPCs), a 50% incidence of myelodysplastic syndrome or acute myeloid leukemia by adolescence, and a 25% lifetime incidence of head and neck squamous cell carcinoma or gynecological cancer. In some patients, blood cell clones demonstrate spontaneous reversion to wild-type (i.e. somatic mosaicism), leading to improved and stable blood cell counts for up to 27 years. Thus, correction of the FA hematopoietic defect could significantly alter the disease's clinical course, which has driven decades of research in HSPC gene therapy for FA.

Initial clinical trials demonstrated a dramatic 50-fold reduction in the number of true HSPCs in FA patients relative to other gene therapy patients, such as those treated for primary immune deficiencies. Moreover, FA HSPCs were exceptionally fragile when manipulated ex vivo for gene transfer. No patient treated demonstrated stable improvements in blood cell counts with long-term persistence of gene-corrected blood cells. These studies highlighted two needs for innovation in FA gene therapy: (1) to increase the number of HSPCs available for collection for gene transfer and infusion, and (2) to increase the engraftment potential of these cells after gene transfer.

Following recommendations of the International FA Gene Therapy Working Group, a phase I clinical trial of gene therapy for FA complementation group A (FA-A) patients was launched in 2014 (National Clinical Trials Registry ID NCT01331018). This trial design incorporates several features aiming to improve HSPC numbers and fitness. These include a self-inactivating (SIN) lentiviral vector (LV) for transfer of the FANCA cDNA regulated by a human phosphoglycerate kinase (hPGK) promoter, a short, overnight transduction to minimize ex vivo manipulation, as well as addition of the antioxidant N-acetylcysteine (NAC) throughout manipulation, and culture under reduced oxygen (5%) to limit oxidative DNA damage.

The target HSPC population for gene transfer expresses the CD34 cell surface protein (CD34⁺). When stained with fluorophore-conjugated antibody against CD34 and analyzed by flow cytometry, a small proportion of BM cells are CD34⁺, representing both primitive stem cells and more committed progenitors. The standard clinical procedure for isolating these cells first involves either BM collection or mobilization of the cells into circulation through cytokine stimulation with granulocyte colony stimulating factor (G-CSF) or a combination of G-CSF and the chemokine receptor CXCR4 antagonist plerixafor, followed by PB leukapheresis (mAPH). Initial isolation technologies relied on CD34 antigen expression on the cell surface and utilized biotin-avidin affinity, panning, or immunomagnetic bead-based approaches. Expected yields were 50% of total CD34+ cells available with highly variable purities, ranging from 20-90% across techniques [reviewed in Collins. Stem Cells. 1994; 12(6):577-585]. Of these, immunomagnetic bead-based positive selection is the most widely-applied at clinical scale today, with the first FDA approval of a clinical device for human use in 2014. Advances in this technology to include automation have improved reliability in recovery to a mean yield of 70% with purities regularly >90% [Spohn et al. Cytotherapy. 2015; 17(10):1465-1471 and Avecilla et al. Transfusion. 2016; 56(5):1008-1012]. However, these values are based on BM and mAPH products wherein 1-3% of total cells express CD34 antigen, and the majority of these cells display high levels of CD34. For FA patients, the frequency of CD34+ cells is much lower, 0.1-1.5% in B M. Muller & Williams. Mutat Res. 2009; 668(1-2):141-149; Kelly et al. Mol Ther. 2007; 15(1):211-219. This implies that non-standard processes may be required to preserve the limited numbers of HSPCs for gene transfer in FA.

Here HSPC collection results for three patients are described. Initially, this protocol proposed direct isolation of CD34+ cells from BM without prior attempts at mobilization, which could exhaust the limited numbers of HSPCs in FA patients. This permitted evaluation of CD34 expression patterns and potential for direct isolation using standard clinical protocols to provide evidence for the need for alternative HSPC isolation strategies.

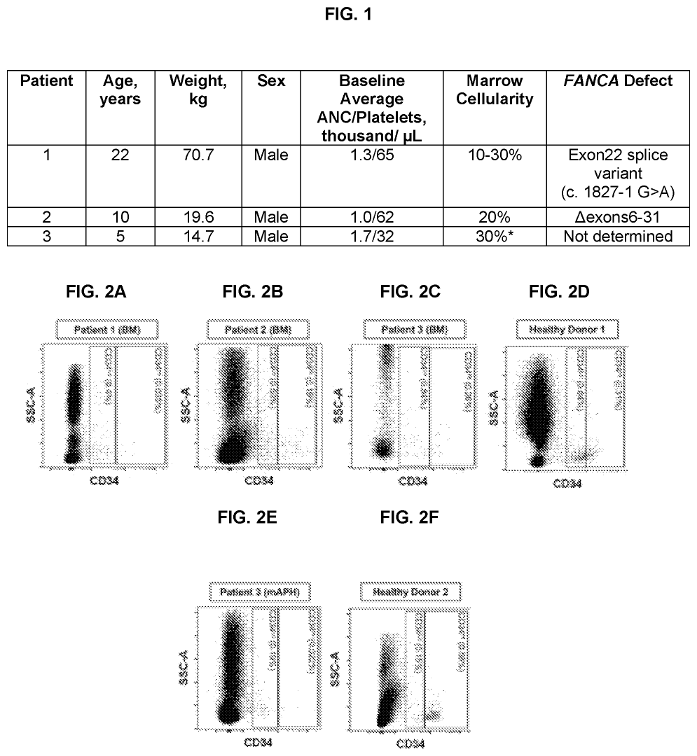

Methods. Patient selection. This study was approved by an Institutional Review Board at Fred Hutchinson Cancer Research Center (Fred Hutch) in accordance with the Declaration of Helsinki and the United States Food and Drug Administration (FDA), and conformed to the National Institutes of Health Guidelines for Research Involving Recombinant DNA Molecules. Informed consent was obtained from all patients. FA patients aged A years were diagnosed by a positive test for increased sensitivity to chromosomal breakage with mitomycin C (MMC) or diepoxybutane. Patients in the A complementation group who demonstrated normal karyotype in BM analyses as defined in the trial were considered eligible for the study. Enrolled patient characteristics are available in FIG. 1.

Lentivirus vectors. All SIN LV vectors were produced with a third-generation split packaging system and pseudotyped with vesicular stomatitis virus glycoprotein (VSVG). LV used to transduce healthy donor cells encoded either an enhanced green fluorescent protein (eGFP) transgene (pRSC-PGK.eGFP-sW) or the full-length FANCA cDNA (pRSC-PGK.FANCA-sVV), both regulated by a human phosphoglycerate kinase (PGK) promoter. In particular embodiments, the PGK promoter has a sequence of SEQ ID NO: 56. Research-grade vectors were produced by the Fred Hutch Vector Production Core. Clinical-grade LV (pRSC-PGK.FANCA-sW), was produced by the Indiana University Vector Production Facility (IUVPF) using a large-scale, validated process following Good Manufacturing Practices standards under an approved Drug Master File held by IUVPF. Infectious titer was determined by serial transduction of HT1080 human fibrosarcoma-derived cells and evaluated either by flow cytometry for eGFP expression or by Taqman qPCR.

Study design and HSPC isolation. Patients underwent either BM harvest with a target collection goal of 15 cc/kg body weight or were administered daily G-CSF (filgrastim; 16 μg/kg BID; days 1-6) and plerixafor (240 μg/kg/day; days 4-6) subcutaneously to mobilize CD34⁺ cells. Mobilized patients were subjected to large volume leukapheresis when circulating CD34⁺ blood cell counts were cells/μL. Healthy donor blood products were purchased from a commercial source (BM products; StemExpress, Folsom, Calif.) or institutional shared resources (mAPH products). Immunomagnetic beads were from Miltenyi Biotech, Gmbh (Auburn, Calif.). For BM products, RBC were debulked by hetastarch sedimentation prior to labeling on a CliniMACS Prodigy™ device (Miltneyi Biotec GmbH, Auburn, Calif.). For mAPH products, an initial platelet wash was performed prior to labeling. Custom programming for lineage depletion was designed and executed on the CliniMACS Prodigy™ device (Miltenyi Biotec GmbH, Auburn, Calif.). Complete processing methods are included in the following sections.

Processing of BM and mAPH products. All processing was conducted in a Class II, Type A2 biological safety cabinet or on a CliniMACS Prodigy™ device (Miltenyi Biotec GmbH, Auburn, Calif.). Prior to device loading, 1 L of PlasmaLyte A (Baxter Healthcare, Deerfield, Ill.) was supplemented with NAC to a final concentration of 1 mM and mixed well. A 3 L bag of CliniMACS (PBS/EDTA) buffer (Miltenyi Biotec GmbH, Auburn, Calif.) was supplemented with 60 mL of 25% human serum albumin (HSA; Baxter Healthcare) and NAC to a final concentration of 1 mM and mixed well. For BM products, a 600 mL bag of 6% hetastarch (HES) in 0.9% NaCl (Hospira Inc., Lake Forest, Ill.), was supplemented with NAC to a final concentration of 1 mM. Immunomagnetic bead reagents were concentrated on the device using a custom program and a TS 510 tubing set (Miltenyi Biotec GmbH, Auburn, Calif.), to a total volume of 14 mL or less and then transferred into a 60 mL syringe containing 3 mL of 10% IVIg (GAMMAGARD; Baxter Healthcare) and 20 mL of air, and stored at 2-8° C. until needed. For device loading, a TS 510 tubing set was installed.

BM products were collected by standard clinical procedures. Upon receipt, complete blood counts were obtained and BM was diluted in PlasmaLyte A+NAC to obtain a hematocrit value of 25% prior to loading into the tubing set Application Bag. To facilitate HES sedimentation, 400 mL funneled cryobags (OriGen Biomedical, Austin, Tex.) were sterile-welded onto the tubing set prior to installation. When more than a single bag was used, bags were joined by sterile-welding onto a color-coded, trifurcated, standard bore extension set (Smiths Medical, Saint Paul, Minn.) prior to attachment to the tubing set.

Large-volume mAPH products were collected by standard clinical procedures on a COBE Spectra or Optia apheresis system (Terumo BCT, Lakewood, Colo.). For mAPH products, first day collections were sampled for complete blood cell counts and diluted in autologous plasma to a concentration of 200×10⁶ cells/mL for overnight storage at 2-8° C. as needed. Upon receipt of second day collection, products were pooled and complete blood cell counts were obtained. During TS 510 tubing set preparation, the provided target and non-target cell bags were removed and replaced with 3 L transfer packs (Fenwal, Lake Zurich, Ill.) to accommodate larger volumes of cell product. mAPH products were loaded onto the tubing set via the Application Bag.

Device Set-Up and Operation:

For all processes, valve configurations were as follows:

### Valve 1: CliniMACS (PBS/EDTA) Buffer+NAC+HSA

### Valve 2: Empty

Valve 3: Concentrated bead reagent+IVIg
Valve 4: Funnel cyrobag(s) (BM products ONLY)
Valve 5: HES+NAC (BM products ONLY)

### Valve 6: Empty

### Valve 7: Empty

Valve 8: Application Bag (standard with tubing set)
Valve 9: Pre-separation column

### Valve 10: Priming bag

Valves 11-16 and 21: Directional valves
Valves 17-18: Centricult unit

### Valve 19: Waste bag

Valve 20: Non-target cell bag
Valve 22: Target cell bag
Valve 23: Intermediate storage bag

### Valve 24: Empty

Once the tubing set is loaded and the program is launched, the user chooses whether the initial product is BM or mAPH. If BM is selected, there are five user inputs required for processing to begin: (1) the volume of diluted BM product loaded (+10 mL for complete loading), (2) the measured post-dilution hematocrit value, (3) the number of stages required for loading (total diluted BM volume divided by 300 mL per stage and rounded up to the nearest whole number), (4) the estimated frequency of cells expressing a lineage marker targeted for depletion (“bead-bound”) cells, and (5) the number of cells in millions loaded into the application bag. Following entry, the device prompts the user to perform several checks of the clamps and connections to ensure device loading was accurate and then the automated process begins. Once started, there is no user interface required until HES sedimentation has completed. The user can select to continue the process or permit longer sedimentation if required. Once sedimentation is complete, the user is prompted to begin RBC removal and enters volumes of RBC which are transferred from the bottom of the funnel cryobags (one at a time if more than one bag is used), until the user is satisfied with the volume of RBCs debulked. Once the user indicates RBS removal is sufficient, the program continues automatically to begin lineage depletion. Once lineage depletion is completed, the target cell product bag contains the desired lineage− cell fraction for transduction and is removed from the device by heat sealing the tubing set.

If mAPH is selected, there are two user inputs required for processing to begin: (1) the number of cells in millions loaded into the application bag, and (2) the estimated frequency of cells expressing a lineage marker targeted for depletion (“bead-bound”) cells. Following entry, the device prompts the user to perform several checks of the clamps and connections to ensure device loading was accurate, and then the automated process begins. The user is prompted to mix the Application Bag during two rinse steps at the start of the automated program. Following this interface, there is no other user interface required until the lineage depletion process is completed. Once lineage depletion is completed, the target cell product bag contains the desired lineage⁻ cell fraction for transduction and is removed from the device by heat sealing the tubing set.

For Patient 1 alone, the RBC-debulked BM product was removed from the CliniMACS Prodigy device and further processed by labeling with CliniMACS anti-CD34 microbeads (Miltenyi Biotech GmbH, Auburn, Calif.) and then purified for CD34+ cells using immunomagnetic positive selection on the CliniMACS Select Plus instrument (Miltenyi Biotech GmbH, Auburn, Calif.) per manufacturer's recommendations. No further enrichment strategy following RBC debulking was applied for the BM product from Patient 2. mAPH product collected on the first day from Patient 3 was held overnight at 4° C. on an orbital shaker in the presence of 1 mM concentration of NAC and pooled with the second collection on the following day for RBC debulking and lineage depletion on the CliniMACS Prodigy device.

Transduction. CD34-enriched cells were cultured on RetroNectin™-coated culture flasks at a density of 1×10⁶ cells/mL and 2.9×10⁵ cells/cm² in StemSpan ACF media (StemCell Technologies, Vancouver, BC), supplemented with 4 μg/mL of protamine sulfate (American Pharmaceutical Partners; APP, East Shaumburg, Ill.), 100 ng/mL each of recombinant human stem cell factor (rhSCF), thrombopoietin (rhTPO) and Flt-3 ligand (rhFLT3L) (CellGenix GmbH, Freiburg, Germany), and 1 mM N-acetylcystine (Cumberland Pharmaceuticals, Nashville, Tenn.). Cells were immediately transduced at a multiplicity of infection (MOI) of 5-10 infectious units (IU)/cell. Following 12-24 hours of incubation at 37° C., 5% CO₂ and 5% O₂, cells were harvested for infusion and/or analyses.

Transplantation in NSG mice. All animal work was performed under protocol 1864 approved by the Fred Hutch Institutional Animal Care and Use Committee. NOD.Cg-PrkdcscidlL2rγtmlWj/Szj (NOD/SCID/IL2rγ^{null}, NSG) mice were housed at Fred Hutch in pathogen-free conditions approved by the American Association for Accreditation of Laboratory Animal Care. Mice 8-12-weeks-old received 275 cGy TBI. Four hours after TBI, 1×10⁶ gene-modified total nucleated cells (TNC) re-suspended in 200 μL phosphate buffered saline (D-PBS, Life Technologies Corporation, Grand Island, N.Y.) containing 1% heparin (APP) were infused via tail vein. Blood samples were collected into ethylenediaminetetraacetic acid (EDTA) Microtainers (BD Bioscience, San Jose, Calif.) by retro-orbital puncture and diluted 1:1 with PBS prior to analysis. At necropsy, spleen and BM were collected. Tissues were filtered through 70 μm mesh (BD Bioscience) and washed with Dulbecco's PBS (D-PBS).

Colony-forming cell assays. Transduced cell products were seeded in standard colony-forming cell (CFC) assays in methylcellulose media (H4230, StemCell Technologies) as previously described (Radtke et al. Sci Transl Med. 2017; 9(414)) with the following exceptions: to assess FANCA gene function, MMC (Sigma Aldrich, St. Louis, Mo.) was added at concentrations of 0 nM, 5 nM, 10 nM, or 20 nM. Complete colony DNA extraction and PCR methods are described in the following section.

Colony-forming assay methods. Colony-forming assays were plated into methylcellulose containing 2% heat inactivated fetal bovine serum (ThermoFisher, Waltham, Mass.) and 100 ng/mL each of the following growth factors: rh-interleukin3 (IL-3), rhIL-6, rh-TPO, rh-erythropoietin (EPO), rhSCF, rh-granulocyte colony stimulation factor (G-CSF) and rh-granulocyte monocyte colony stimulating factor (GM-CSF) [IL-3, IL-6 and TPO were purchased from PeproTech, EPO, SCF, and G-CSF were purchased from Amgen Inc., Thousand Oaks, Calif., and GM-CSF was purchased from Miltenyi Biotech GmbH, Auburn, Calif.]. Cells were cultured for 12-14 days until robust colony development was achieved and evaluated by colony count and phenotype as granulocyte-monocyte (GM) colonies distinguished as dense white colonies with a halo appearance, erythroid (E) colonies distinguished by a distinct red coloration due to hemoglobinization, or mixed (GEMM) colonies distinguished by a mixture of hemoglobinized and white colonies. Individual colonies were also picked into QuickExtract DNA Extraction Solution 1.0 (Epicenter, Lucigen Corporation, Middleton, Wis.). Genomic DNA was isolated by heating at 65° C. for 20 minutes and then at 99° C. for 10 minutes to quench the enzymatic reaction. DNA was analyzed by PCR using primers specific to LV backbone (forward, 5′-AGAGATGGGTGCGAGAGCGTCA (SEQ ID NO: 57); reverse, 5′-TGCCTTGGTGGGTGCTACTCCTAA (SEQ ID NO: 58)). The reaction mix was subjected to PCR conditions of 94° C. for 2 minutes for initial denaturation followed by 38 cycles of 94° C. for 1 minute (denaturation), 65° C. for 0.5 minutes (annealing) and 72° C. (extension), and finally 72° C. for 10 minutes (final extension). DNA samples were also run in a separate reaction for β-actin as an internal DNA control (forward, 5′-TCCTGTGGCATCGACGAAACT (SEQ ID NO: 59); reverse, 5′-GAAGCATTTGCGGTGGACGAT (SEQ ID NO: 60)). This reaction mix was subjected to PCR conditions of 95° C. for 2 minutes for initial denaturation, followed by 37 cycles of 95° C. for 1 minute (denaturation), 62° C. for 1 minute (annealing) and 72° C. (extension), and finally 72° C. for 10 minutes (final extension). Colonies containing expected bands for both LV and β-actin were scored positive for transduction. Reactions which did not yield β-actin products were considered non-evaluable.

Quantitative real-time PCR-based measurement of vector copy number. Vector copy number (VCN) per genome equivalent was assessed by TaqMan 5′ nuclease quantitative real-time PCR assay in duplicate reactions with a LV-specific primer/probe combination (forward, 5′-TGAAAGCGAAAGGGAAACCA (SEQ ID NO: 61); reverse, 5′-CCGTGCGCGCTTCAG (SEQ ID NO: 62); probe, 5′-AGCTCTCTCGACGCAGGACTCGGC (SEQ ID NO: 63; Integrated DNA Technologies; IDT, Coralville, Iowa)) and in a separate reaction with a β-globin-specific primer/probe combination (forward, 5′-CCTATCAGAAAGTGGTGGCTGG (SEQ ID NO: 64); reverse, 5′-TTGGACAGCAAGAAAGTGAGCTT (SEQ ID NO: 65); probe, 5′-TGGCTAATGCCCTGGCCCACAAGTA (SEQ ID NO: 66) (IDT, Coralville, Iowa)). Two standard curves were established by serial dilution of gDNA isolated from a human cell line (HT1080) confirmed to contain a single integrant of the same LV backbone and from peripheral leukocytes collected from a healthy donor using both primer-probe sets independently.

Individual colony gDNA samples were subjected to multiplex real-time Taqman qPCR to amplify the LV-specific product and an endogenous control (Taqman Copy Number Reference assay RNaseP, Thermo Fisher Scientific, Pittsburgh, Pa.). Samples with an average VCN 0.5 were considered transduced.

Flow cytometry analysis of hematopoietic subsets. Stained cells were acquired on a FACS Canto II, FACS Aria II or FACS LSR II (all from BD Biosciences, San Jose, Calif.) and analyzed using FlowJo software v10.0.8 (Tree Star Inc., Ashland, Oreg.). Analysis was performed on up to 20,000 cells. Gates were established using full minus one stained controls.

Antibodies included anti-human CD34 (clone 563), CD16 (clone 3G8), CD3 (clone UCHT1), CD4 (clone L200), CD8 (clone RPA-T8), all from BD Biosciences, San Jose, Calif.; CD14 (clone 61D3, Thermo Fisher Scientific, Pittsburgh, Pa.); CD19 (clone 4G7, BD Pharmingen, San Diego, Calif.); CD90 (clone 5E10), CD20 (clone 2H7), CD15 (clone W6D3), all from Biolegend (San Diego, Calif.); CD133 (clone 293C3, Miltenyi Biotec GmbH, Auburn, Calif.); CD45 (clone D058-1283) and CD45RA (clone 5H9), both from BD Horizon (San Jose, Calif.).

For mouse samples, antibodies were anti-mouse CD45-V500 (561487, clone 30-F11), anti-human CD45-PerCP (347464, clone 2D1), CD3-FITC (555332, clone UCHT1), CD4-V450 (560345, clone RPA-T4), CD8-APCCy7 (557834, clone SK1), CD20-PE (555623, clone 2H7), and CD14-APC (555824, clone 581), all from BD Biosciences, San Jose, Calif.

Results. Diminished CD34^{Hi} expressing cells in FA-A BM and mAPH. Two enrolled patients underwent BM harvest to collect available CD34⁺ HSPCs (Patients 1 and 2). The third patient underwent mobilization with filgrastim and plerixafor followed by PB leukapheresis (Patient 3). All three patients demonstrated reduced CD34 expression and estimated numbers of CD34⁺ cells in screening BM aspirates prior to collection and treatment, relative to healthy donor BM products, as well as in cell products collected for CD34⁺ cell isolation and gene transfer (FIGS. 2A-2F). Two levels of CD34 expression were observed, CD34^{Lo} (mean fluorescence intensity (MFI)=3,512±627), and CD34^{Hi} (MFI=20,070±5,008). Notably, the proportion of CD34^{Hi} cells were markedly reduced in FA-A patients relative to those observed in healthy donors (FIGS. 2A-2F).

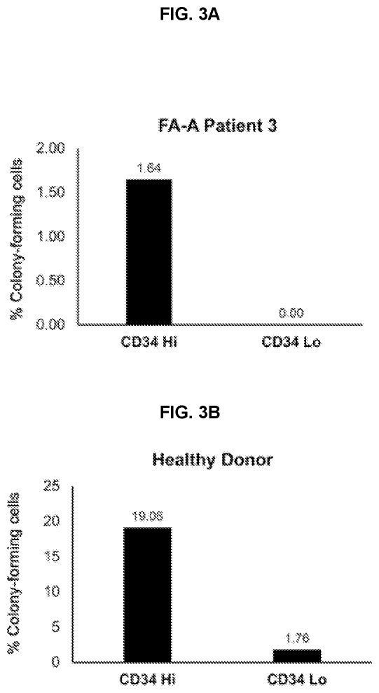

FA-A CD34^{Hi} cells, but not CD34^{Lo} cells, demonstrate in vitro repopulating capacity. To determine which CD34⁺ cells demonstrated repopulation potential, CFC potential was used as a surrogate. This required sufficient blood product to flow-sort CD34^{Lo} and CD34^{Hi} cells for in vitro assays. Only the mAPH product collected from Patient 3 was sufficient for this study. For direct comparison, CD34^{Lo} and CD34^{Hi} cells were sort-purified from a healthy donor mAPH product. Only CD34^{Hi} cells from the FA-A patient demonstrated colony-forming potential (FIG. 3A). In the healthy donor, CD34^{Hi} cells also demonstrated higher CFC capacity in comparison with CD34^{Lo} cells and at much higher levels as compared to the FA-A patient (FIG. 3B). These data suggest repopulating capacity is restricted to CD34^{Hi} cell fractions, underscoring the need to preserve as many of these cells as possible for gene transfer processes.

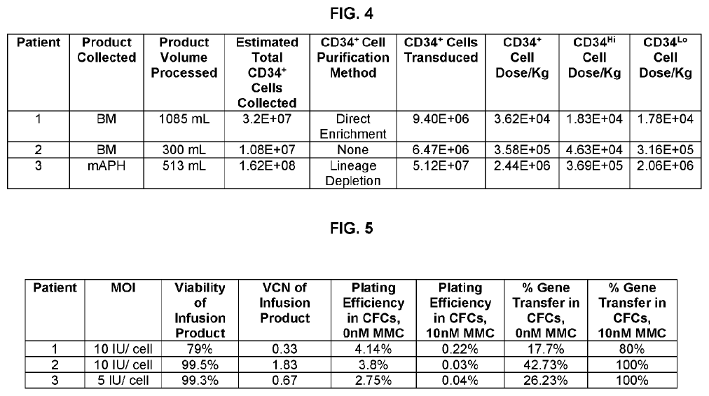

Extensive loss of FA-A CD34^{Hi} cells with current clinical purification protocols. The current clinical standard for CD34⁺ cell enrichment is optimized for collection of CD34^{Hi} cells. However, in Patient 1, direct enrichment of CD34⁺ cells using this protocol was inefficient, resulting in a 29% yield and only 9.4×10⁶ total CD34⁺ cells available for gene transfer (FIG. 4). Moreover, the purity of the enriched cell product was only 59.6%, and 50% loss in viable cells was observed during culture and gene transfer. Resulting gene-modified cells retained colony-forming capacity and demonstrated acquired resistance to MMC following LV-mediated FANCA gene transfer (FIG. 5).

In Patient 2, estimated losses during direct CD34 enrichment and gene transfer were expected to reduce the cell product available for transduction to a lower level than observed for Patient 1. Thus, an urgent amendment was filed with the FDA to permit elimination of the direct CD34 enrichment steps and allow transduction of the entire red blood cell (RBC)-depleted BM product. While this processing change preserved more CD34⁺ cells (FIG. 4), with improved transduction and viability (FIG. 5), it required a prohibitive volume (80 mL) of concentrated, clinical-grade LV vector. Together, these data suggested that minimal manipulation of target CD34⁺ cells from FA-A patients could improve yield, gene transfer efficiency, and function in vivo, but would require reduced numbers of non-target cells to achieve feasible LV usage.

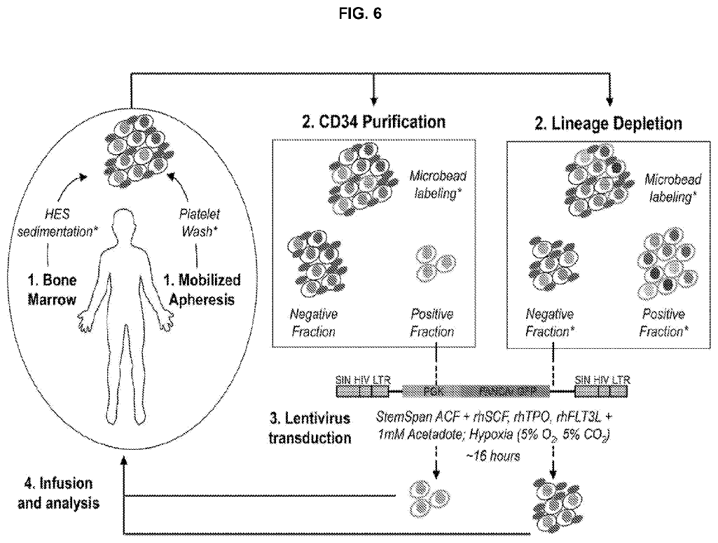

Development of a novel strategy to deplete lineage⁺ cells. It was hypothesized that depleting non-target mature B cells, T cells, monocytes, and granulocytes would reduce the amount of LV required to transduce a BM cell product while retaining precious CD34⁺ cells with minimal manipulation, since CD34-expressing cells would not be directly labeled, selected, or washed (FIG. 6). Building on previous work automating cell selection and gene transfer using the CliniMACS Prodigy™ device (Adair et al. Nat Commun. 2016; 7:13173), a customized, automated RBC debulking and immunomagnetic bead-based lineage depletion strategy was designed. Four different bead-conjugated antibody reagents were used in this approach: anti-CD3 (T cell removal), anti-CD14 (monocyte removal), anti-CD16 (granulocyte and NK cell removal), and anti-CD19 (B cell removal). This protocol was designed for both BM and mAPH products.

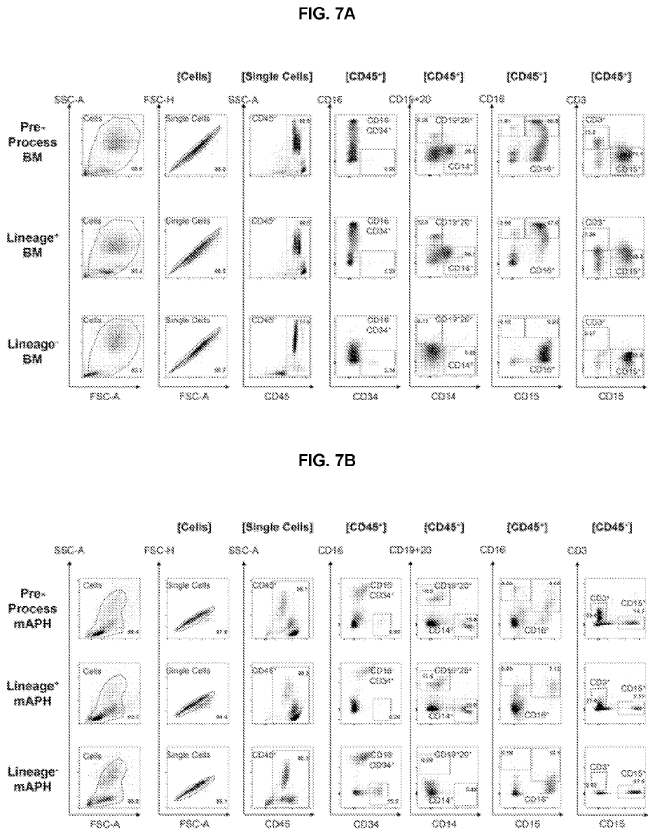

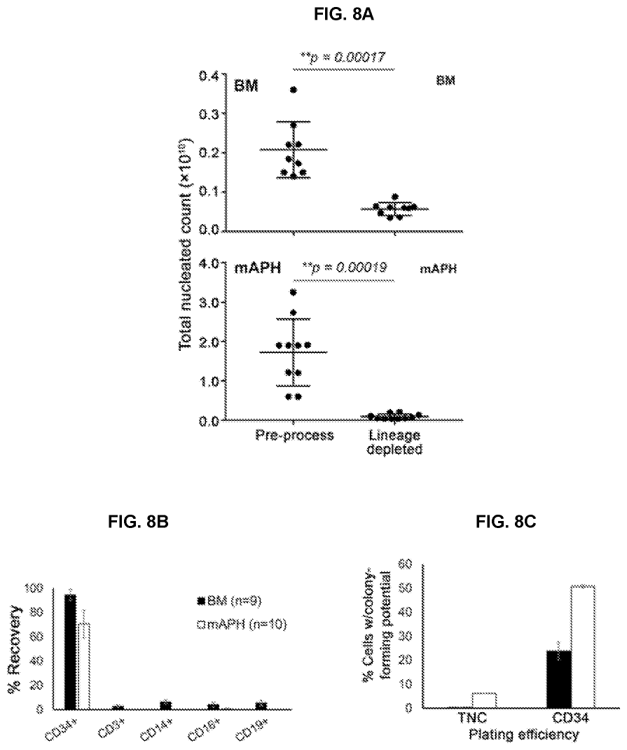

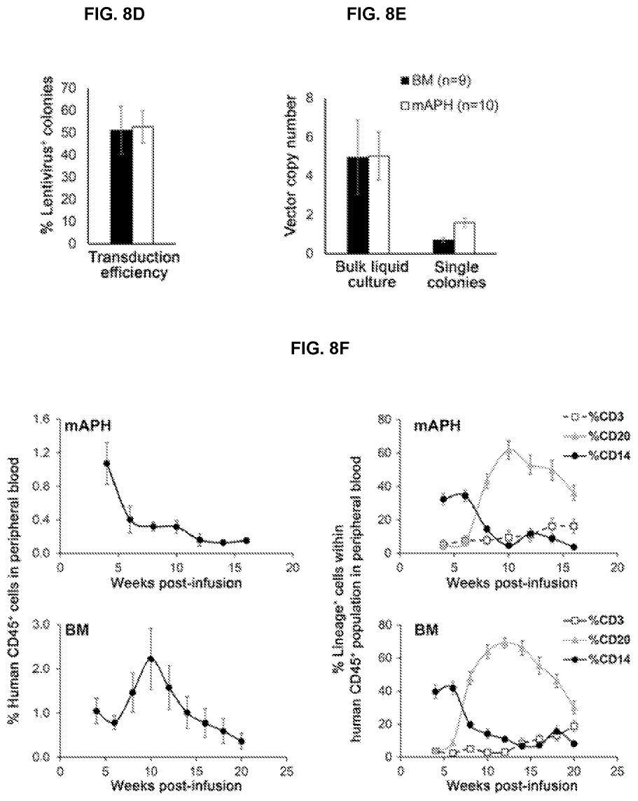

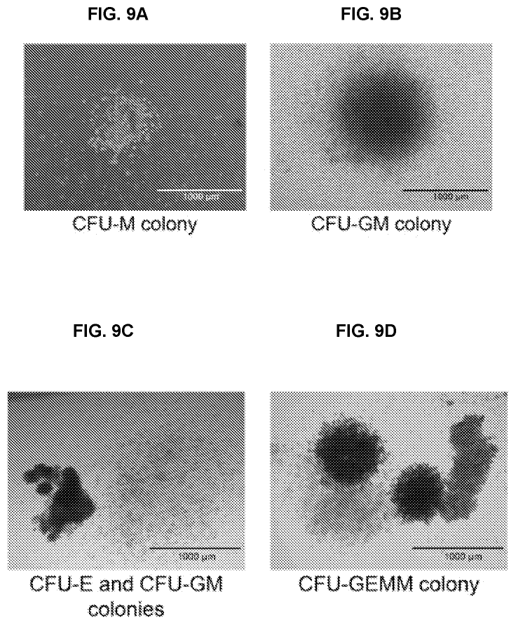

Lineage depletion preserves available CD34⁺ cells and reduces TNC to feasible numbers for LV-mediated gene transfer. A total of nine BM and ten mAPH products were processed to establish process validity. An average 60% of BM CD45⁺ cells and 50% of mAPH CD45⁺ cells expressed one of the four target markers (CD3, CD14, CD16, or CD19) (FIG. 7A and FIG. 7B, respectively). CD34⁴ cell content in these products ranged from 0.35%-1.4% in BM and 0.06%-0.9% in mAPH products. The average process run time for BM products was 10 hours, whereas mAPH products were processed over 13 hours. Observed TNC reduction was 1 log for both BM and mAPH products following lineage depletion (FIG. 8A). All target lineage⁺ cells were depleted to <10% of initial, and CD34⁺ cells were retained at >90% for BM products and >70% for mAPH products (FIG. 8B). 24% of BM CD34⁺ cells were colony-forming in a standard methylcellulose assay, while 51% of mAPH CD34⁺ cells formed colonies (FIG. 8C, FIGS. 9A-9D). However, following LV transduction of these cells using the same protocol proposed for FA-A patient cells, consistent 50% rates of gene transfer into CFCs from both cell product types was observed (FIG. 8D). Analysis of single colonies demonstrated an average VCN per CFC of 0.7 for BM CD34⁺ cells and 1.6 for mAPH CD34⁺ cells. VCN was also assessed in bulk transduced cells cultured for 10 days in vitro, demonstrating an average value of 5 for both BM and mAPH products (FIG. 8E). Final cell products tested for Mycoplasma and sterility were negative, and endotoxin testing demonstrated values within criteria for patient infusion. Lineage-depleted and transduced cells from six mAPH and BM products each were infused into NSG mice at a target cell dose of 1×10⁶ TNC per mouse. On average, the CD34⁺ cells dose per mouse for BM products was 2.86×10⁴ CD34⁺ cells (SEM=6.67×10³) and for mAPH products was 1.08×10⁶ CD34⁺ cells (SEM=1.45×10⁴). Flow cytometry analysis on PB WBCs was used to evaluate engraftment (human CD45⁺) and lineage development into T cells (human CD3⁺), B cells (human CD20⁺), and monocytes (human CD14⁺) over time (FIG. 8F). Both mAPH and BM products demonstrated long-term engraftment over 20 weeks of monitoring. Engraftment levels were comparable to results reported by Wiekmeijer et al. (Biores Open Access. 2014; 3(3):110-116) with CD34⁺ cells purified from BM and infused at similar cell doses.

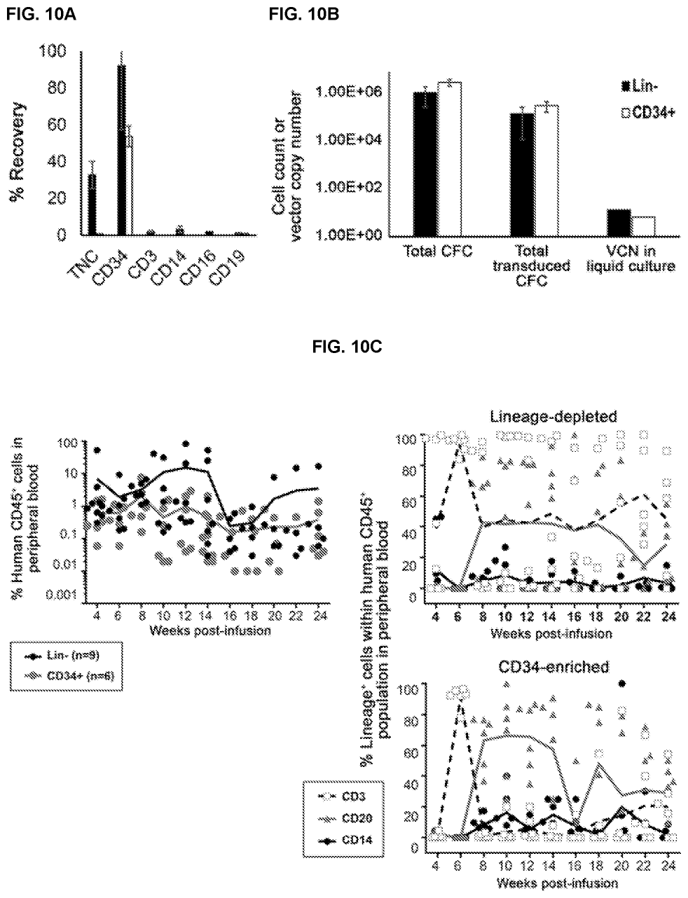

Lineage-depleted cell products xenoengraft equivalently to CD34-enriched products. In this example, healthy donor BM products were divided into two aliquots. One was lineage depleted and the other CD34-enriched. Resulting cell populations were transduced with the same LV vector under identical conditions and infused into NSG mice at matched CD34⁺ cell doses. While higher CD34⁺ cell retention was observed with lineage depletion compared to CD34 selection, no other differences were observed in transduction efficiency or colony-forming potential (FIGS. 10A, 10B). Slightly higher, but not significantly different, levels of human CD45⁺ blood cell engraftment in mice receiving transduced, lineage-depleted cells relative to mice receiving CD34-selected cells were observed. More stability of T and B cell engraftment in mice receiving lineage-depleted cell products relative to mice receiving CD34-selected cell products was also observed (FIG. 100).

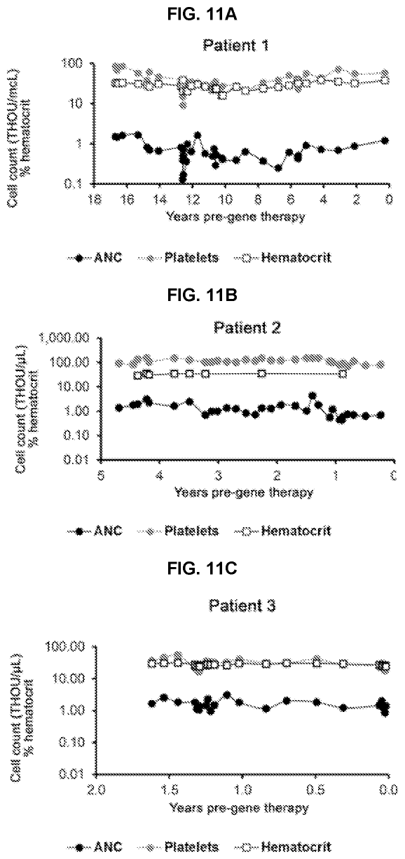

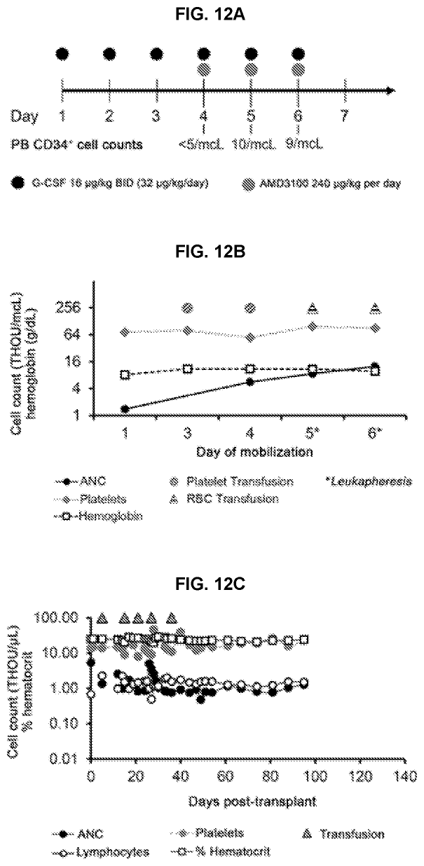

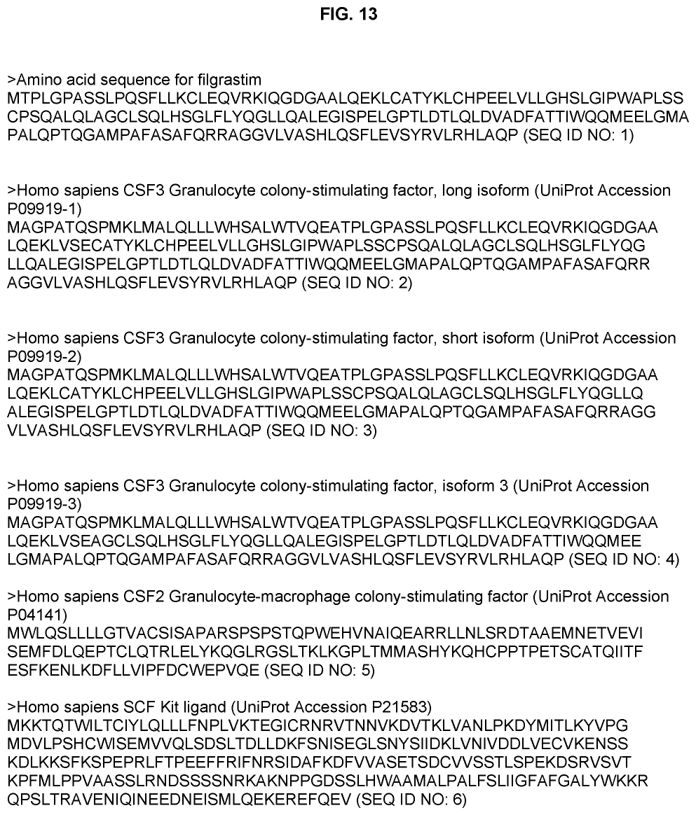

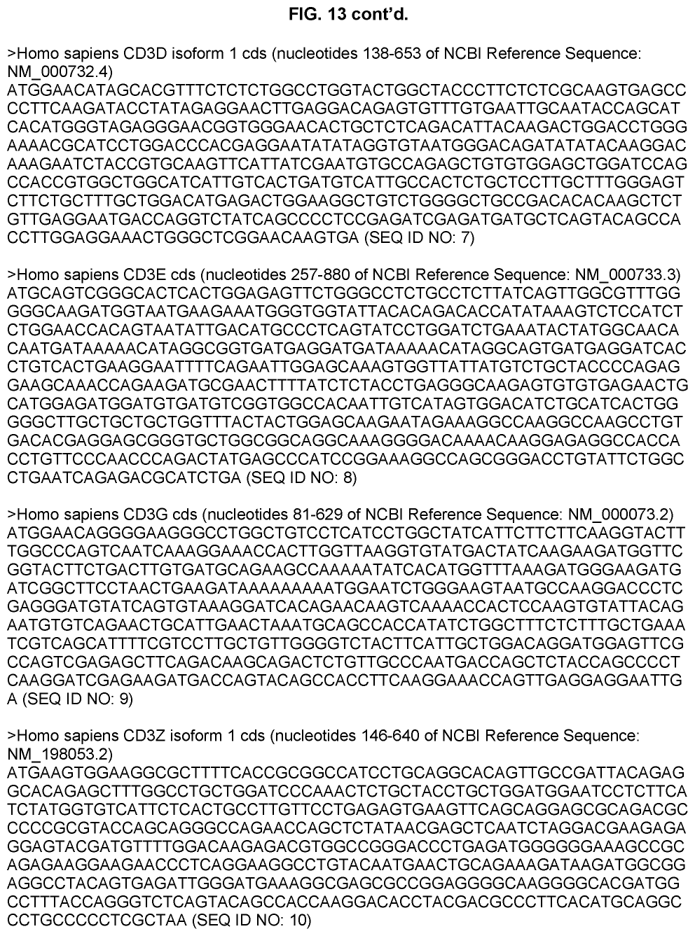

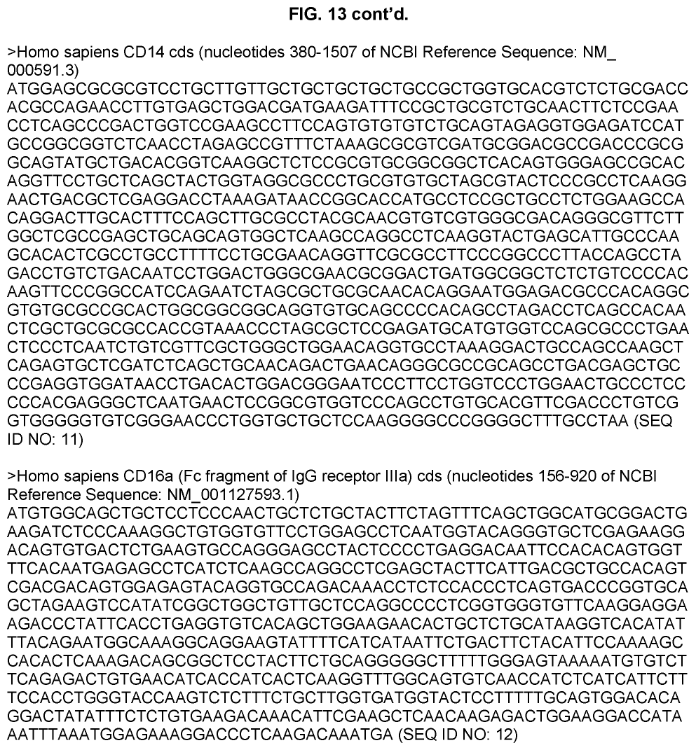

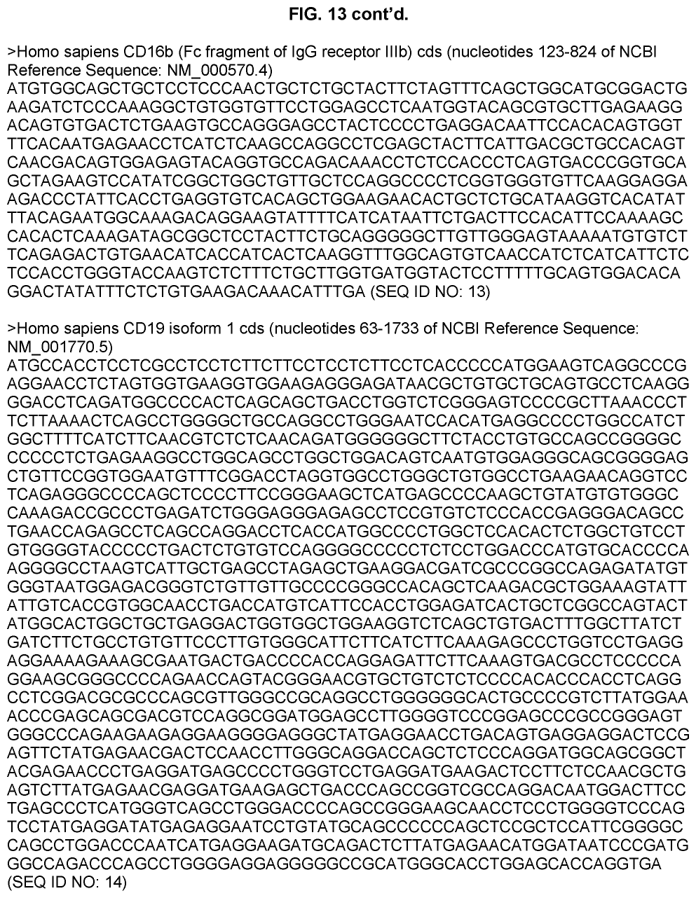

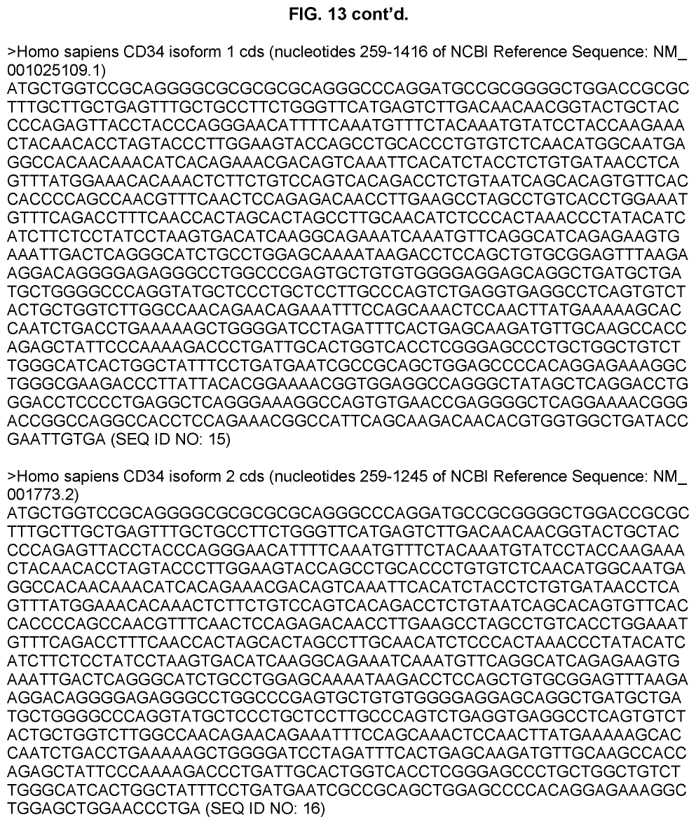

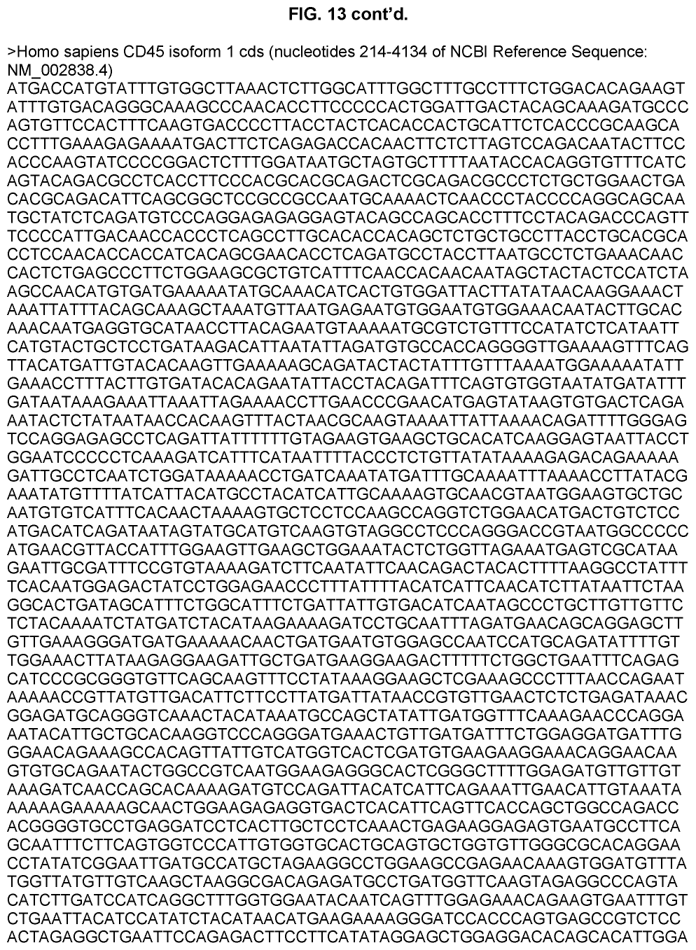

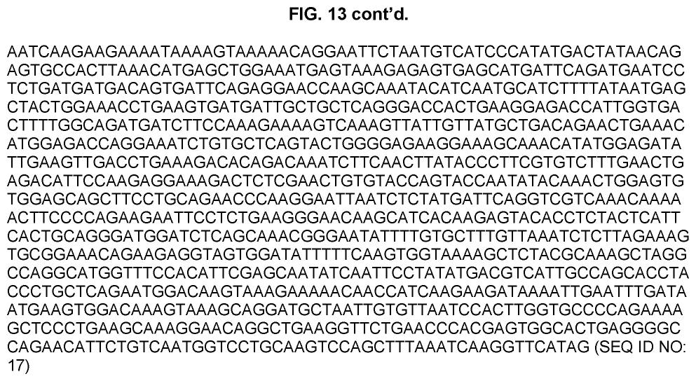

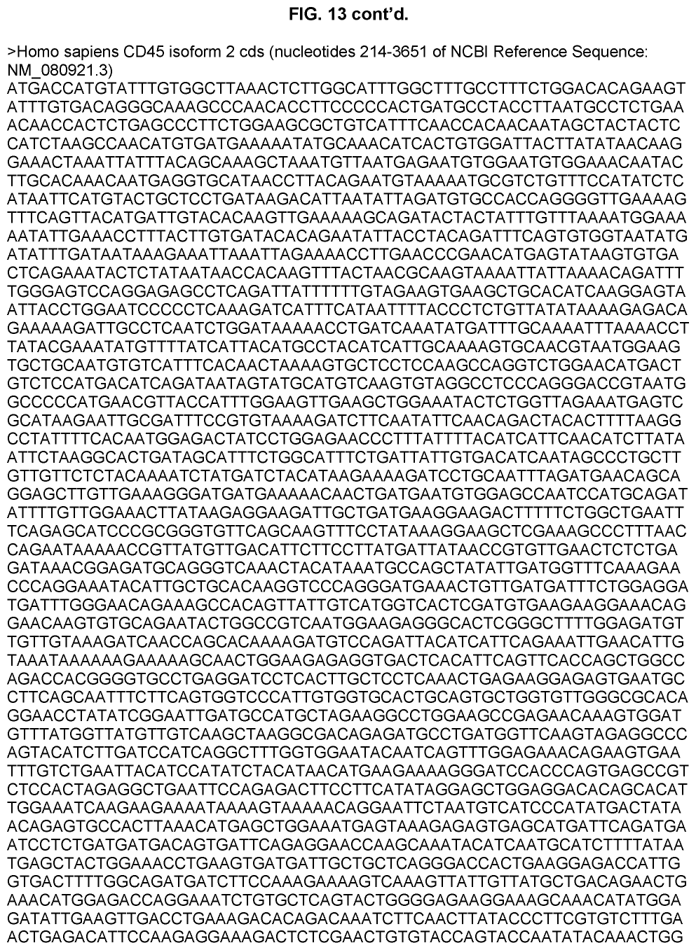

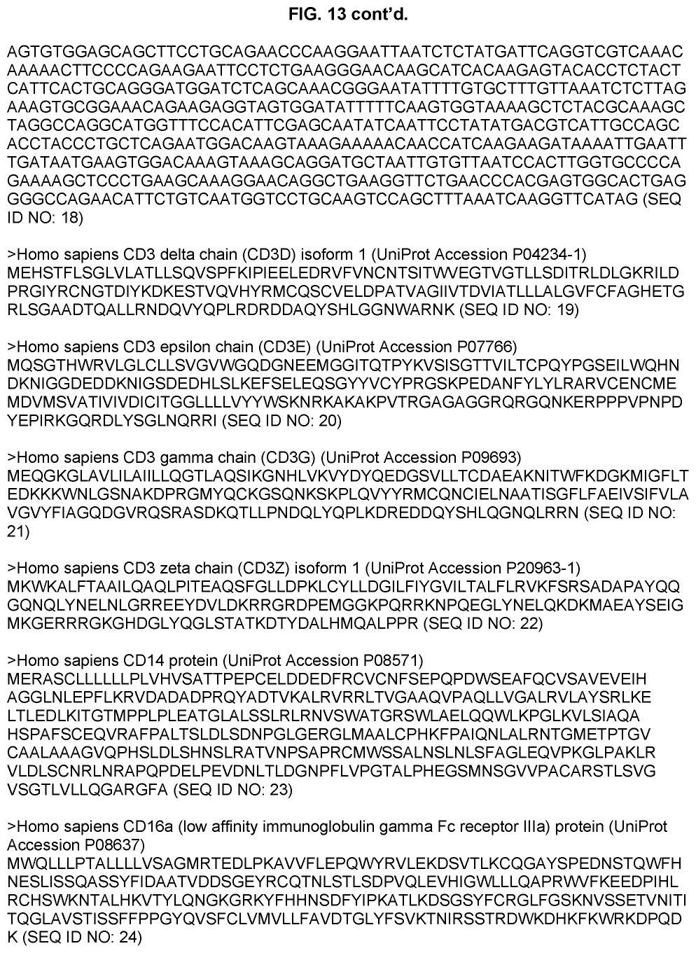

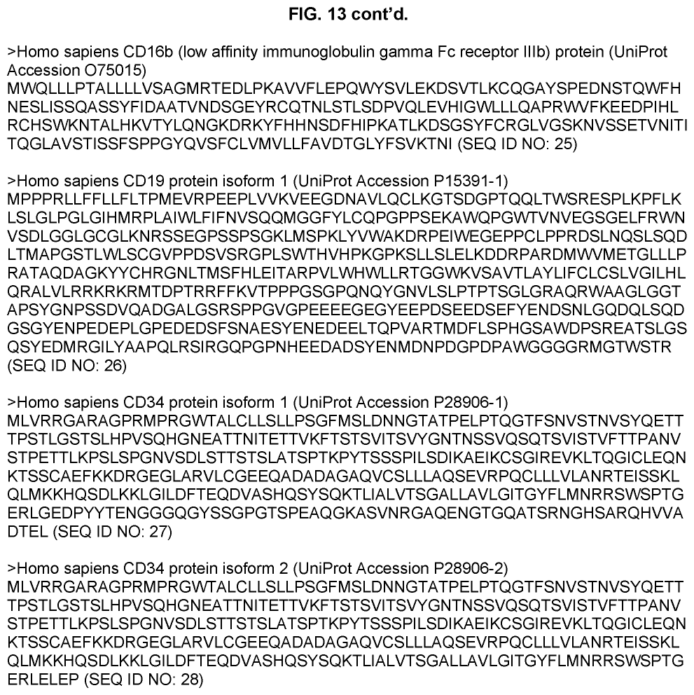

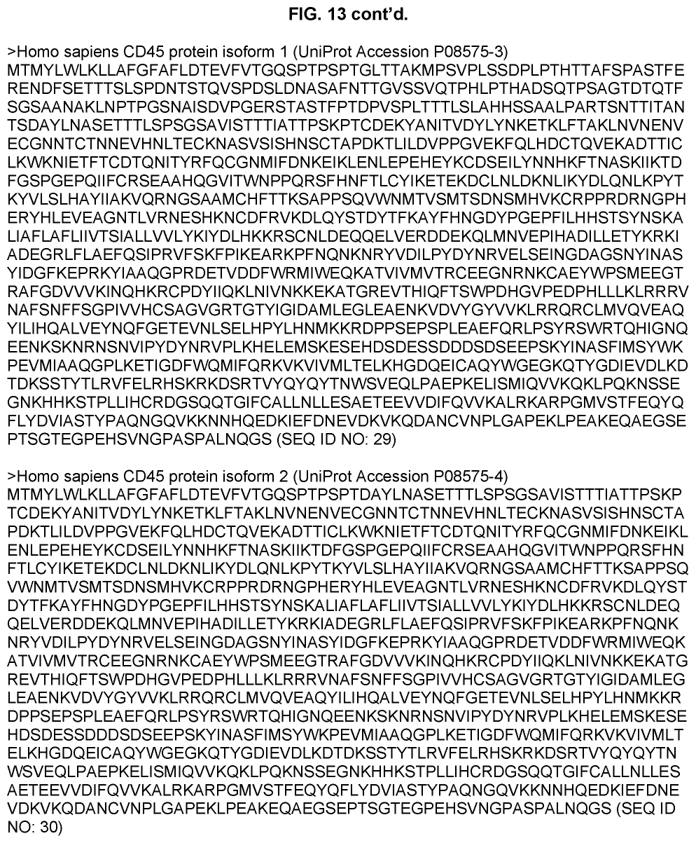

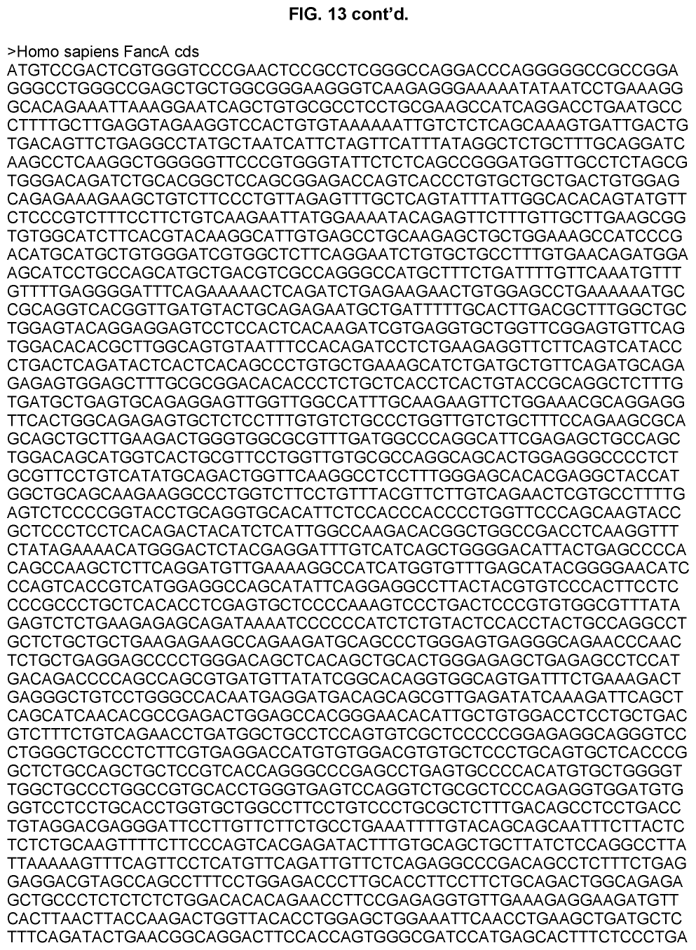

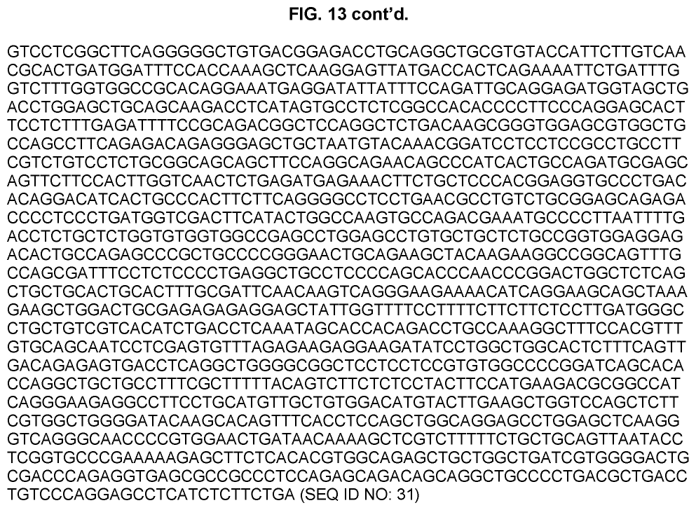

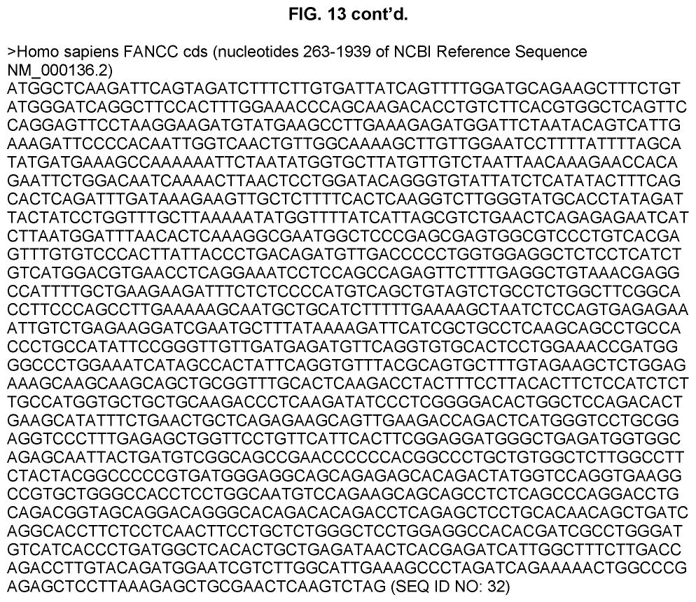

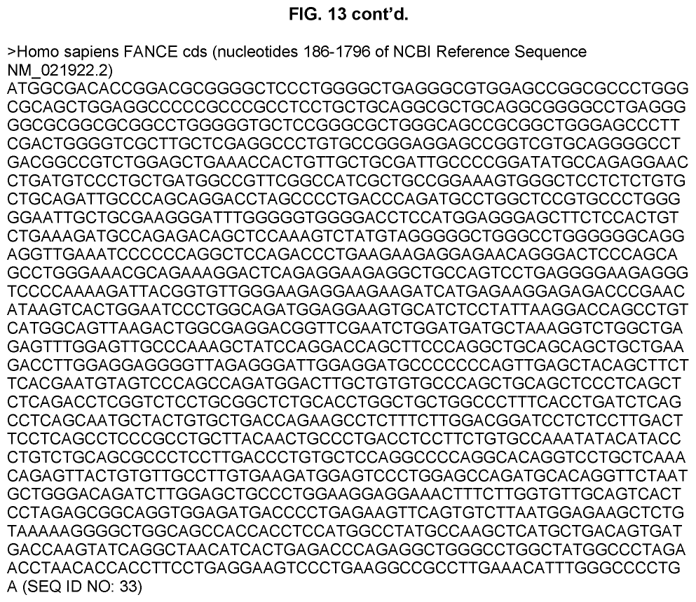

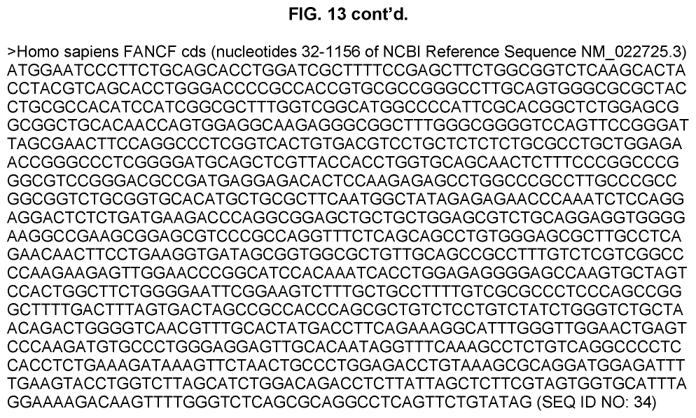

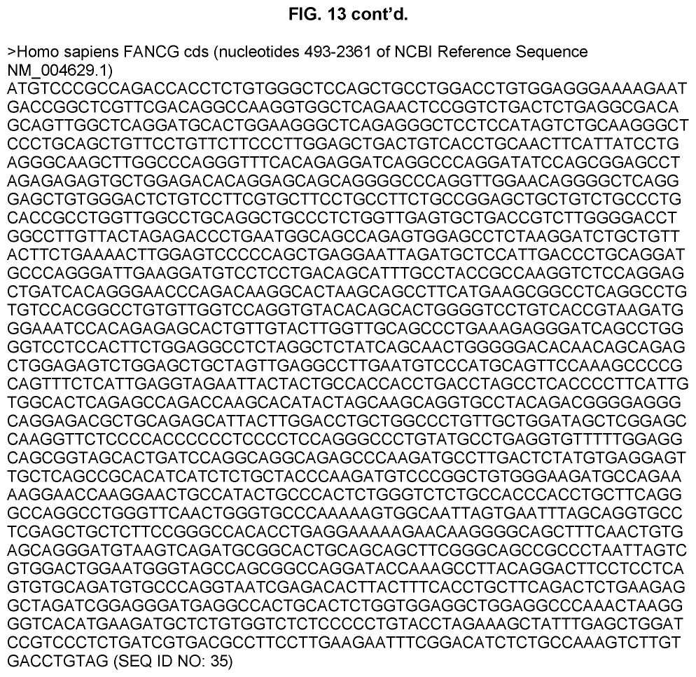

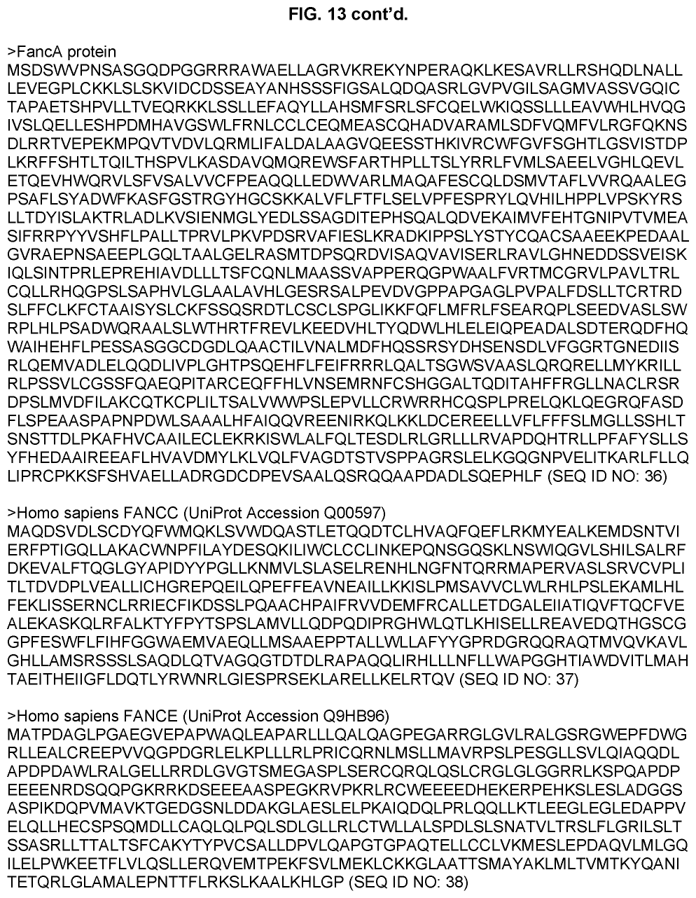

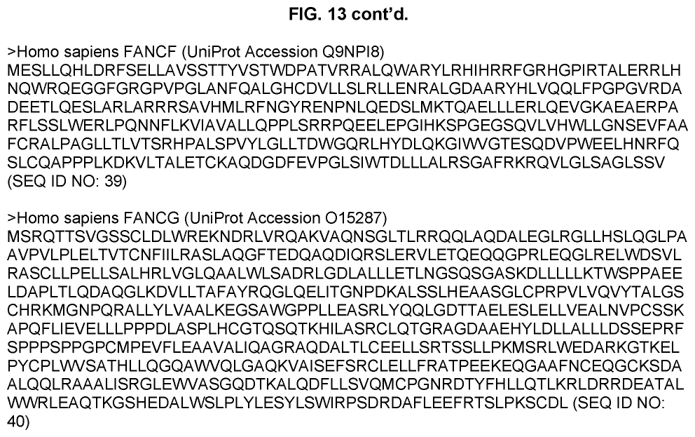

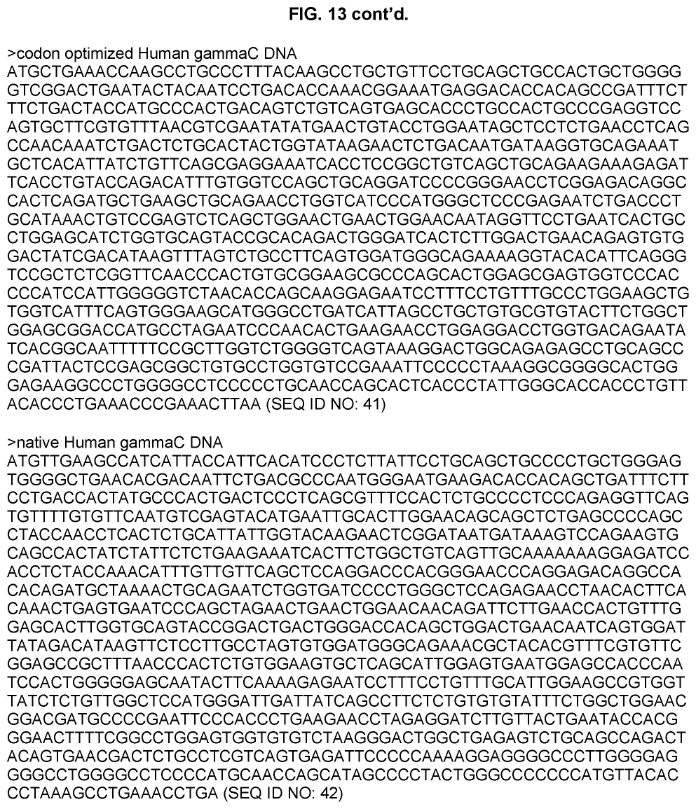

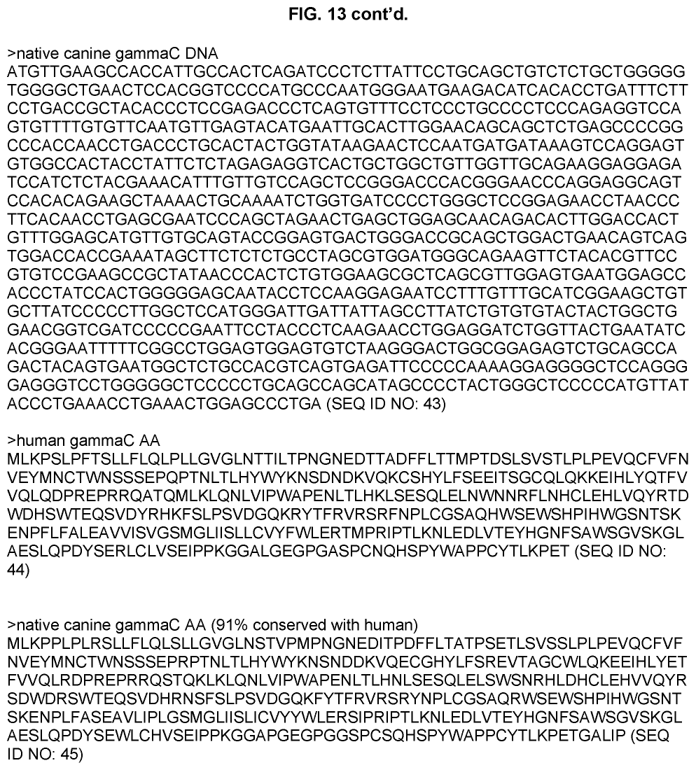

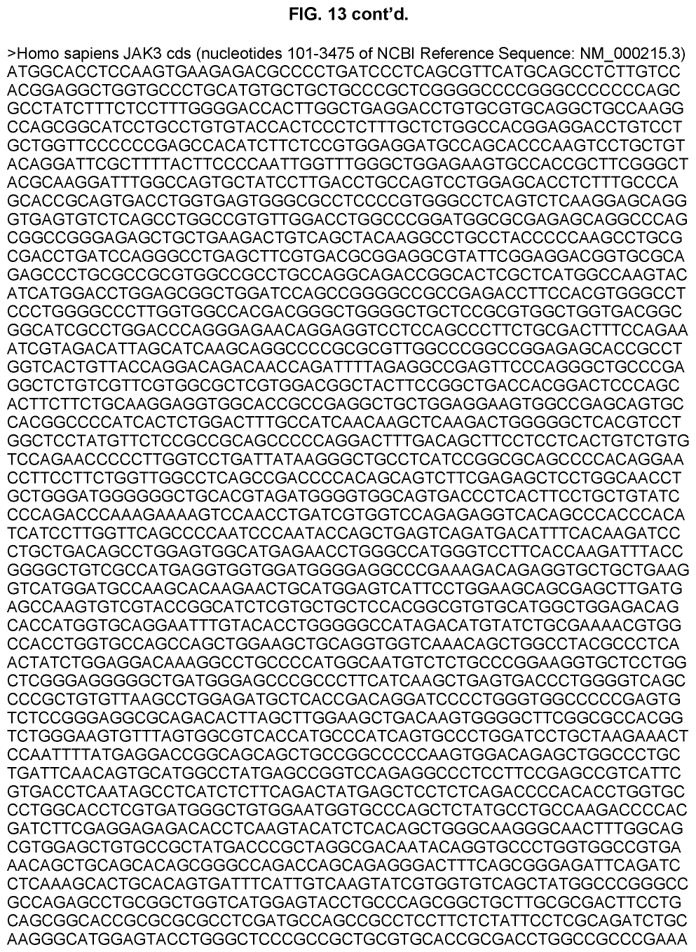

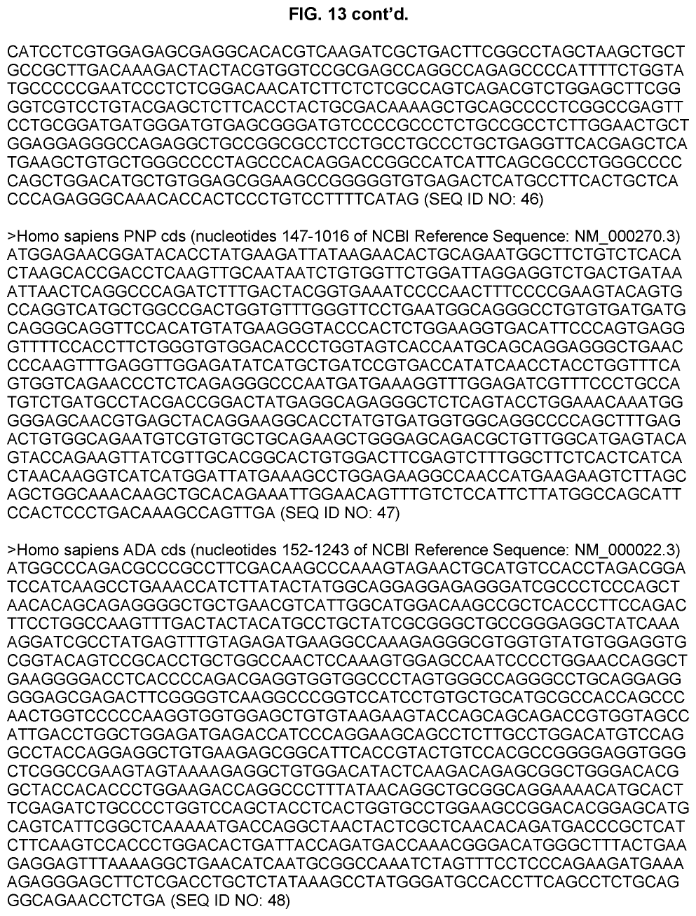

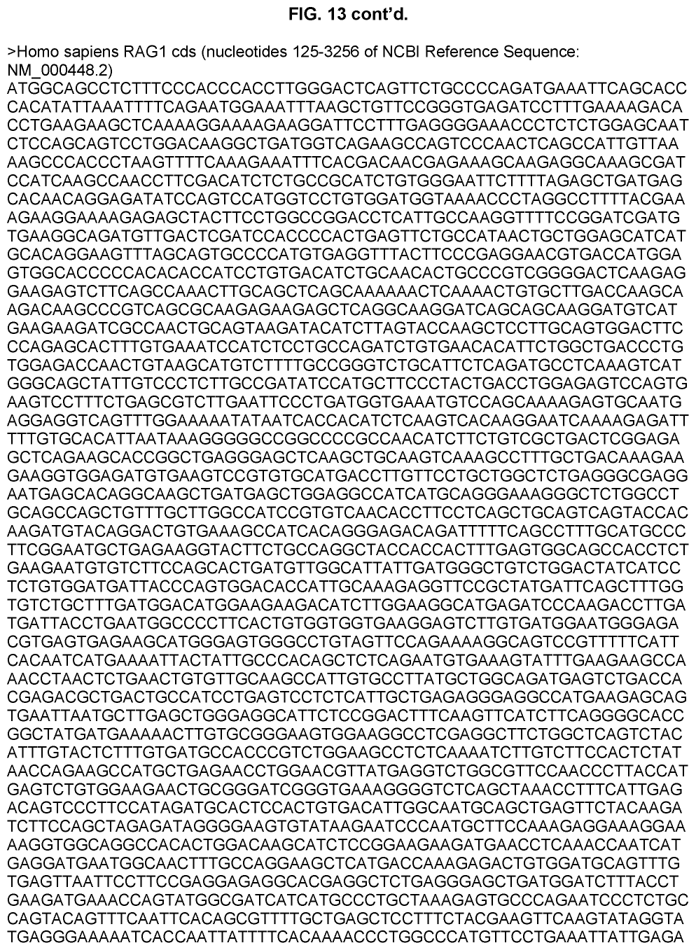

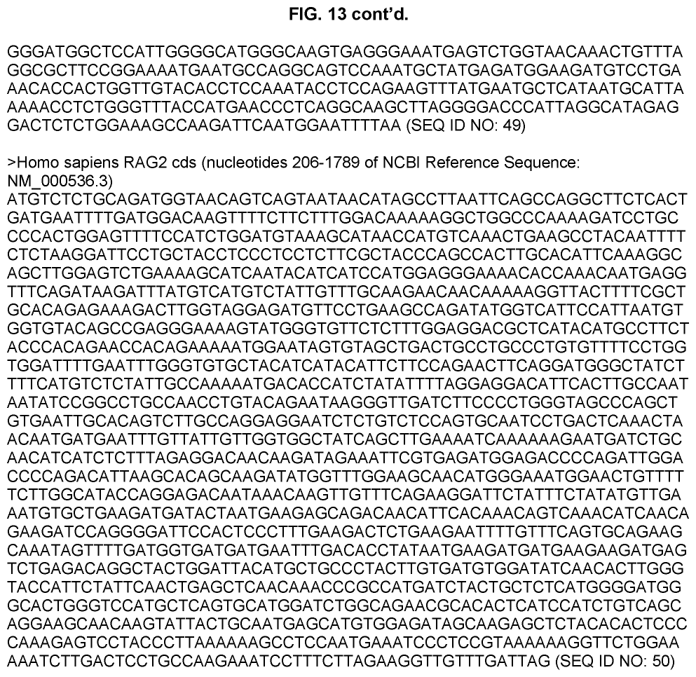

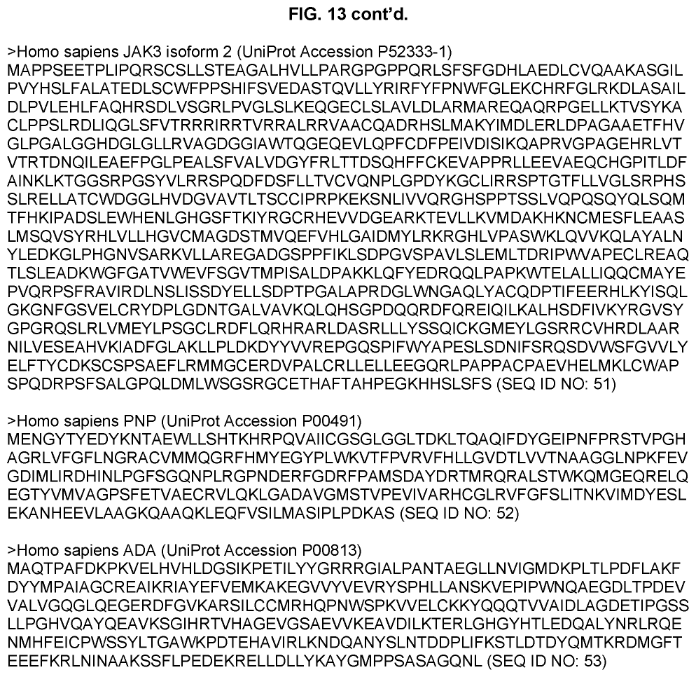

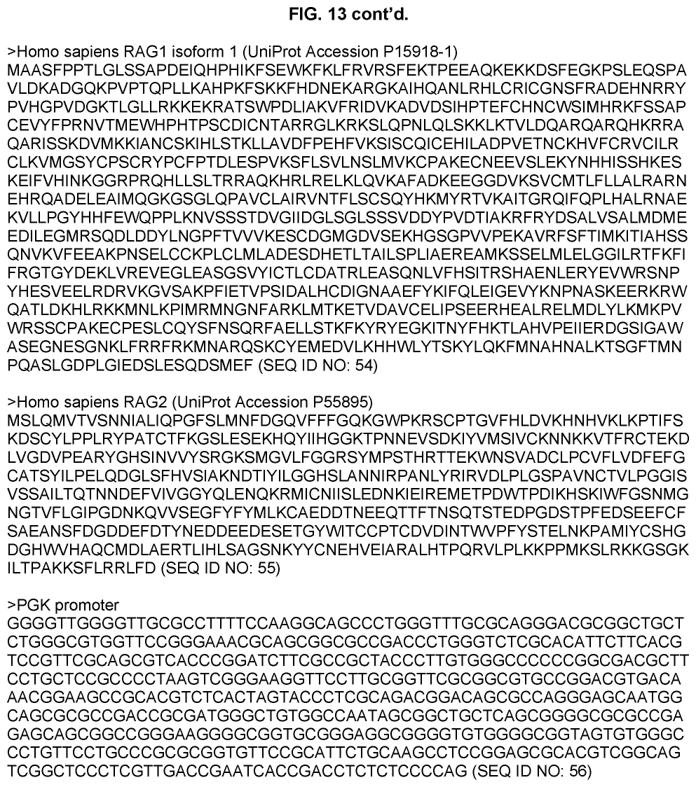

Lineage depletion protocol preserves limited FA CD34^{Hi} cells. These data collectively suggest that lineage depletion preserved available CD34⁺ cells and reduced TNC counts to feasible numbers for transduction without compromising transduction efficiency or cell fitness. Under FDA approval, the clinical protocol was modified to include both BM and mAPH products, with lineage depletion as the method of CD34⁺ cell enrichment. Patient 3, the first treated under the modified protocol, was a 5-year-old male with confirmed FA-A by complementation studies. Baseline neutrophils averaged 1.7K/mcL and baseline platelets averaged 32K/mcL in the 6 months prior to treatment, with declining neutrophils and platelets over the prior 2-year interval (FIGS. 11A-11C). Mobilization of ≥10 CD34+ cells/μL PB was achieved, and two successive apheresis collections resulted in 8.5×10¹⁰ TNC containing a total 1.6×10⁸ CD34⁺ cells (FIGS. 4 and 12A). Due to column limitations, 5×10¹⁰ TNC (equivalent to 9.5×10⁷ total CD34⁺ cells), were subjected to lineage depletion, and the remainder were cryopreserved. Lineage depletion resulted in a 94% reduction in TNC and a 56% retention of available CD34⁺ cells. CD34 purity was 1.6%, representing a 1-2 log-fold increase in the total number of CD34⁺ cells per kg available for transduction and infusion relative to Patients 1 and 2 (FIG. 4). A total of 52.8×10⁶ CD34⁺ cells were transduced at 5 IU/cell, resulting in a final cell dose of 2.4×10⁶ total CD34⁺ cells per kg with 99.3% viability based on trypan blue dye exclusion. 26% of CFCs in this cell product were transduced, displaying a mean VCN of 1 (0.9) (FIG. 5). All other infusion criteria were met; however, a false positive Mycoplasma result using the MycoAlert™ rapid test was observed and confirmed by a negative qPCR assay result. Thus, limited CD34⁺ cells were indirectly enriched using lineage depletion on a blood product from an FA-A patient without compromising transduction efficiency.

The cell product was reinfused without complication. The patient required a total of two platelet transfusions and two packed RBC transfusions during mobilization and leukapheresis (FIG. 12B). The patient received an additional five platelet transfusions beginning at 5 days post-infusion and ending 36 days after infusion (FIG. 12C). An additional two packed RBC transfusions were administered 15 and 28 days following infusion for low hematocrit.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a decrease in the ability of the systems and methods described herein to achieve negative selection of CD34⁺ cells.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method comprising:
identifying a subject with a fragile CD34+ hematopoietic stem cell (HSC) population;
obtaining a biological sample comprising fragile CD34+ cells from the subject;
removing only CD3+, CD14+, CD16+ and CD19+ cells from the biological sample;
genetically-modifying CD34+ cells remaining within the biological sample by inserting a viral vector comprising a therapeutic gene into genomes of the remaining CD34+ cells; and
formulating the genetically-modified CD34+ cells for administration to the subject.

2. A method of claim 1, wherein the subject is a human patient with Fanconi anemia.

3. A method of claim 1, wherein the removing results in at least a 70%, at least an 80%, or at least a 90% reduction in CD3+ cells, CD14+ cells, CD16+ cells, and CD19+ cells, collectively.

4. A method of claim 1, wherein the method retains at least 50%, at least 70%, or at least 90% of starting CD34+ cells in the biological sample.

5. A method of claim 1, wherein the retained CD34+ cells are CD34+, CD45RA−, and CD90+.

6. A method of claim 1, wherein mesenchymal stem cells remain in the biological sample after the removing.

7. A method of claim 1, wherein the therapeutic gene comprises FancA, FancB, FancC, FancD1, FancD2, FancE, FancF, FancG, FancI, FancJ, FancL, FancM, FancN, FancO, FancP, FancQ, FancR, FancS, FancT, FancU, FancV, or FancW.

8. A method of claim 1, wherein the removing comprises contacting the biological sample with binding proteins consisting of a binding protein that binds CD3, a binding protein that binds CD14, a binding protein that binds CD16, and a binding protein that binds CD19.

9. A method of claim 8, wherein the binding proteins comprise antibodies or binding fragments thereof.

10. A method of claim 8, wherein the binding proteins are coupled to magnetic beads, fluorophores, and/or affinity tags.

11. A method of claim 1, wherein the removing comprises performing magnetic separation, fluorescence activated cell sorting (FACs), nanosorting, affinity chromatography, panning, and/or selective agglutination.

12. A method of claim 9, wherein the antibodies are selected from clone 5H9, clone JS-83, clone HI100, anti-CD45RA [MEM-56], clone UCHT1, clone SK7, clone OKT3, clone CD3-12, clone 61D3, clone HCD14, clone 63D3, clone M5E2, clone 3G8, clone LNK16, clone DJ130c, clone KD1, clone 4G7, clone HIB19, clone LT19, and clone FMC63.

13. A method of claim 12, wherein the antibodies consist of clone 5H9, clone UCHT1, clone 61D3, clone 3G8, and clone 4G7.

14. A method of claim 1, wherein the genetic modification includes transducing a viral vector at a multiplicity of infection (MOI) of 5 to 10 infectious units (IU)/cell.

15. A method of claim 14, wherein the viral vector is a lentiviral vector.

16. A method of claim 15, wherein the lentiviral vector is a self-inactivating lentiviral vector.

17. A method of claim 15, wherein the lentiviral vector includes a human phosphoglycerate kinase (PGK) promoter.

18. A method of claim 17, wherein the human PGK promoter includes SEQ ID NO: 56.

19. A method of claim 1, wherein the biological sample is a bone marrow sample or a mobilized peripheral blood sample.

20. A method comprising forming a CD34+ cell population with accessory cells for a therapeutic or experimental purpose utilizing negative selection, the method comprising:
obtaining a bone marrow sample or a mobilized peripheral blood sample;
removing CD45RA+, CD3+, CD14+, CD16+ and/or CD19+ cells from the biological sample;
thereby forming a CD34+ cell population with accessory cells for a therapeutic or experimental purpose utilizing negative selection.

21. A method of claim 20, wherein the method comprising removing only cells CD45RA+.

22. A method of claim 20, wherein the method comprising removing CD3+, CD14+, CD16+, and CD19+ cells.

23. A method of claim 20, wherein the method comprising removing CD3+, CD14+, and CD19+ cells.

24. A method of claim 20, wherein the method comprising removing only CD3+, CD14+, CD16+, and CD19+ cells.

25. A method of claim 20, wherein the accessory cells are mesenchymal stem cells.

26. A method of claim 20, wherein the removing results in at least a 70%, at least an 80%, or at least a 90% reduction in CD3+ cells, CD14+ cells, CD16+ cells, and CD19+ cells, collectively.

27. A method of claim 20, wherein the method retains at least 50%, at least 70%, or at least 90% of starting CD34+ cells in the biological sample.

28. A method of claim 27, wherein the retained CD34+ cells are CD34+, CD45RA−, and CD90+.

29. A method of claim 20, further comprising genetically-modifying CD34+ cells within the CD34+ cell population.

30. A method of claim 29, wherein the genetically modifying comprises transducing a lentiviral vector.

31. A method of claim 30, wherein the lentiviral vector is a self-inactivating lentiviral vector.

32. A method of claim 30, wherein the lentiviral vector comprises a sequence selected from SEQ ID NOs: 31-35, 41-43, and 46-50.

33. A method of claim 30, wherein the lentiviral vector comprises a sequence encoding a protein selected from SEQ ID NOs: 36-40, 44, 45, and 51-55.

34. A method of claim 30, wherein the lentiviral vector comprises a therapeutic gene.

35. A method of claim 34, wherein the therapeutic gene comprises FancA, FancB, FancC, FancD1, FancD2, FancE, FancF, FancG, FancI, FancJ, FancL, FancM, FancN, FancO, FancP, FancQ, FancR, FancS, FancT, FancU, FancV, FancW, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, SLC46A1, F8, F9, IDUA, iduronidase, IDS, GNS, HGSNAT, SGSH, NAGLU, GUSB, GALNS, GLB1, ARSB, HYAL1, 101F6, 123F2, 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fms, FOX, FUS 1, FUS1, FYN, G-CSF, GDAIF, Gene 21, Gene 26, GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1, LUCA-2, LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, WT1, WT-1, YES, zac1, α2β1, αvβ3, αvβ5, αvβ63, BOB/GPR15, Bonzo/STRL-33/TYMSTR, CCR2, CCR3, CCR5, CCR8, CD4, CD46, CD55, CXCR4, aminopeptidase-N, HHV-7, ICAM, ICAM-1, PRR2/HveB, HveA, α-dystroglycan, LDLR/α2MR/LRP, PVR, PRR1/HveC, laminin receptor, soluble CD40, CTLA, Fas L, globin family genes, WAS, phox, dystrophin, pyruvate kinase, CLN3, ABCD1, arylsulfatase A, SFTPB, SFTPC, NLX2.1, ABCA3, GATA1, ribosomal protein genes, TERT, TERC, DKC1, TINF2, CFTR, LRRK2, PARK2, PARK7, PINK1, SNCA, PSEN1, PSEN2, APP, SOD1, TDP43, FUS, ubiquitin 2, C9ORF72, and/or antibodies to CD4, CD5, CD7, CD52, IL-1, IL-2, IL-6; TNF; P53, PTPN22, DRB1*1501/DQB1*0602or a TCR specifically present on autoreactive T cells; IL-4; IL-10; IL-12; IL-13; IL-1Ra, sIL-1RI, sIL-1 RII; sTNFRI; and/or sTNFRII.

36. A method of claim 20, wherein the removing comprises contacting the biological sample with binding proteins comprising a binding protein that binds CD45RA, a binding protein that binds CD3, a binding protein that binds CD14, a binding protein that binds CD16, and/or a binding protein that binds CD19.

37. A method of claim 36, wherein the binding proteins comprise antibodies or binding fragments thereof.

38. A method of claim 36, wherein the binding proteins are coupled to magnetic beads, fluorophores, and/or affinity tags.

39. A method of claim 20, wherein the removing comprises performing magnetic separation, fluorescence activated cell sorting (FACs), nanosorting, affinity chromatography, panning, and/or selective agglutination.

40. A method of claim 37, wherein the antibodies comprise clone 5H9, clone JS-83, clone HI100, anti-CD45RA [MEM-56], clone UCHT1, clone SK7, clone OKT3, clone CD3-12, clone 61D3, clone HCD14, clone 63D3, clone M5E2, clone 3G8, clone LNK16, clone DJ130c, clone KD1, clone 4G7, clone HIB19, clone LT19, and/or clone FMC63.

41. A method of claim 40, wherein the antibodies comprise clone 5H9, clone UCHT1, clone 61 D3, clone 3G8, and/or clone 4G7.

42. A method of claim 20, further comprising removing red blood cells from the biological sample before the removing.

43. A method of claim 20, wherein the biological sample is a bone marrow sample and the method comprises diluting the bone marrow sample in cell culture media comprising N-acetylcysteine (NAC) to obtain a hematocrit value of 25%.

44. A method of claim 20, further comprising culturing CD34+ cells retained in the biological sample after negative selection in culture media supplemented with protamine sulfate, stem cell factor, thrombopoietin, Flt-3 ligand, and N-acetylcysteine.

45. A method of claim 20, further comprising formulating the CD34+ cells for administration to a subject.

46. A method comprising:
identifying a subject with a fragile CD34+ hematopoietic stem cell (HSC) population, a CD34+I° HSC population, a primary immune deficiency, a secondary immune deficiency, and/or cancer;
obtaining a biological sample comprising CD34+ cells from the subject;
removing CD45RA+, CD3+, CD14+, CD16+ and/or CD19+ cells from the biological sample; and
genetically-modifying CD34+ cells remaining within the biological sample by inserting a nucleic acid encoding a therapeutic gene within the genome.

47. A method of claim 46, further comprising formulating the genetically-modified CD34+ cells for administration to the subject.

48. A method of claim 46, wherein subject is a human patient that has Fanconi anemia, sickle cell disease (SCD), or Dyskeratosis congenita.

49. A method of claim 46, wherein the secondary immune deficiency is caused by HIV.

50. A method of claim 46, wherein the cancer is a leukemia.

51. A method of claim 46, wherein the removing results in at least a 70%, at least an 80%, or at least a 90% reduction in CD3+ cells, CD14+ cells, CD16+ cells, and CD19+ cells, collectively.

52. A method of claim 46, wherein the method retains at least 50%, at least 70%, or at least 90% of starting CD34+ cells in the biological sample.

53. A method of claim 52, wherein the retained CD34+ cells are CD34+, CD45RA−, and CD90+.

54. A method of claim 46, wherein mesenchymal stem cells remain in the biological sample after the removing.

55. A method of claim 46, wherein the genetically modifying comprises transducing a viral vector at a multiplicity of infection (MOI) of 5 to 10 infectious units (IU)/cell.

56. A method of claim 46, wherein the genetically modifying comprises transducing a lentiviral vector.

57. A method of claim 56, wherein the lentiviral vector is a self-inactivating lentiviral vector.

58. A method of claim 56, wherein the lentiviral vector comprises a sequence selected from SEQ ID NOs: 31-35, 41-43, and 46-50.

59. A method of claim 56, wherein the lentiviral vector comprises a sequence encoding a protein selected from SEQ ID NOs: 36-40, 44, 45, and 51-55.

60. A method of claim 46, wherein the removing comprises contacting the biological sample with binding proteins comprising a binding protein that binds CD45RA, a binding protein that binds CD3, a binding protein that binds CD14, a binding protein that binds CD16, and/or a binding protein that binds CD19.

61. A method of claim 60, wherein the binding proteins comprise antibodies or binding fragments thereof.

62. A method of claim 60, wherein the binding proteins are coupled to magnetic beads, fluorophores, and/or affinity tags.

63. A method of claim 46, wherein the removing comprises performing magnetic separation, fluorescence activated cell sorting (FACs), nanosorting, affinity chromatography, panning, and/or selective agglutination.

64. A method of claim 61, wherein the antibodies comprise clone 5H9, clone JS-83, clone HI100, anti-CD45RA [MEM-56], clone UCHT1, clone SK7, clone OKT3, clone CD3-12, clone 61D3, clone HCD14, clone 63D3, clone M5E2, clone 3G8, clone LNK16, clone DJ130c, clone KD1, clone 4G7, clone HIB19, clone LT19, and/or clone FMC63.

65. A method of claim 64, wherein the antibodies comprise clone 5H9, clone UCHT1, clone 61 D3, clone 3G8, and clone 4G7.

66. A therapeutic cell formulation comprising a CD34+ cell retained in a biological sample following the negative selection of any of the preceding claims.

67. A therapeutic cell formulation comprising a CD34+ cell retained in a biological sample following the negative selection of any of the preceding claims and comprising a genetic modification of any of the preceding claims.

68. A kit comprising:
one or more proteins that bind CD45RA, CD3, CD14, CD16, and/or CD19;
culture media; and
a viral vector comprising a therapeutic gene selected from FancA, FancB, FancC, FancD1, FancD2, FancE, FancF, FancG, FancI, FancJ, FancL, FancM, FancN, FancO, FancP, FancQ, FancR, FancS, FancT, FancU, FancV, FancW, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, SLC46A1, F8, F9, IDUA, iduronidase, IDS, GNS, HGSNAT, SGSH, NAGLU, GUSB, GALNS, GLB1, ARSB, HYAL1, 101F6, 123F2, 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fms, FOX, FUS 1, FUS1, FYN, G-CSF, GDAIF, Gene 21, Gene 26, GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1, LUCA-2, LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, WT1, WT-1, YES, zac1, α2β1, αvβ3, αvβ5, αvβ63, BOB/GPR15, Bonzo/STRL-33/TYMSTR, CCR2, CCR3, CCR5, CCR8, CD4, CD46, CD55, CXCR4, aminopeptidase-N, HHV-7, ICAM, ICAM-1, PRR2/HveB, HveA, α-dystroglycan, LDLR/α2MR/LRP, PVR, PRR1/HveC, laminin receptor, soluble CD40, CTLA, Fas L, globin family genes, WAS, phox, dystrophin, pyruvate kinase, CLN3, ABCD1, arylsulfatase A, SFTPB, SFTPC, NLX2.1, ABCA3, GATA1, ribosomal protein genes, TERT, TERC, DKC1, TINF2, CFTR, LRRK2, PARK2, PARK7, PINK1, SNCA, PSEN1, PSEN2, APP, SOD1, TDP43, FUS, ubiquilin 2, C9ORF72, and/or antibodies to CD4, CD5, CD7, CD52, IL-1, IL-2, IL-6; TNF; P53, PTPN22, DRB1*1501/DQB1*0602or a TCR specifically present on autoreactive T cells; IL-4; IL-10; IL-12; IL-13; IL-1Ra, sIL-1RI, sIL-1RII; sTNFRI; and/or sTNFRII.

69. A kit of claim 68, wherein the proteins that bind consist of proteins that bind CD45RA.

70. A kit of claim 68, wherein the proteins that bind consist of proteins that bind CD3, CD14, CD16, and CD19.

71. A kit of claim 68, wherein the proteins that bind consist of proteins that bind CD3, CD14, and CD19.

72. A kit of claim 68, wherein the therapeutic gene comprises FancA, FancB, FancC, FancD1, FancD2, FancE, FancF, FancG, FancI, FancJ, FancL, FancM, FancN, FancO, FancP, FancQ, FancR, FancS, FancT, FancU, FancV, and FancW.

73. A kit of claim 68, wherein the kit comprises one or more of Protamine sulfate; Human stem cell factor; Thrombopoietin; Flt-3 ligand; and N-acetylcysteine.

74. A kit of claim 68, comprising mobilization factors.

75. A kit of claim 74, wherein the mobilization factors are selected from G-CSF, GM-CSF, SCF, AMD3100, and a chemotherapeutic agent.

76. A kit of claim 68, wherein the binding proteins comprise clone 5H9, clone JS-83, clone HI100, anti-CD45RA [MEM-56], clone UCHT1, clone SK7, clone OKT3, clone CD3-12, clone 61D3, clone HCD14, clone 63D3, clone M5E2, clone 3G8, clone LNK16, clone DJ130c, clone KD1, clone 4G7, clone HIB19, clone LT19, and/or clone FMC63. comprise clone 5H9, clone UCHT1, clone 61D3, clone 3G8, and/or clone 4G7.

77. A kit of claim 68, further comprising magnetic beads, fluorophores, and/or affinity tags.

78. A kit of claim 68, further comprising medical supplies to obtain a bone marrow sample or a mobilized peripheral blood sample.

79. A kit of claim 68, further comprising a tubing set.

80. A kit of claim 68, wherein the viral vector is a lentiviral vector.

81. A kit of claim 80, wherein the lentiviral vector is a self-inactivating lentiviral vector.

82. A kit of claim 68, further comprising media to formulate cells for administration to a subject.
